(19) United States
(12) Patent Application Publication
(10) Pub. No.: US 2015/0240304 A1
(43) Pub. Date: Aug. 27, 2015

(54) GENES AND GENES COMBINATIONS BASED ON GENE MKNK1 PREDICTIVE OF EARLY RESPONSE OR NON RESPONSE OF SUBJECTS SUFFERING FROM INFLAMMATORY DISEASE TO CYTOKINE TARGETING DRUGS (CYTD) OR ANTI-INFLAMMATORY BIOLOGICAL DRUGS

(71) Applicants: Alessandra Cervino, Nantes (FR); Oana Popa-Nita, Nantes (FR); Jonathan Plassais, Lyon (FR)

(72) Inventors: Alessandra Cervino, Nantes (FR); Oana Popa-Nita, Nantes (FR); Jonathan Plassais, Lyon (FR)

(21) Appl. No.: 13/981,792

(22) PCT Filed: Jan. 25, 2012
(86) PCT No.: PCT/EP2012/051164

§ 371 Date: Oct. 20, 2014

Related U.S. Application Data
(60) Provisional application No. 61/457,191, filed on Jan. 25, 2011.
(60) Provisional application No. 61/457,743, filed on May 25, 2011.
(60) Provisional application No. 61/588,390, filed on Jan. 19, 2012.

(51) Int. Cl. C12Q 1/68; A61K 39/395; C07K 16/24; A61K 31/519

(52) U.S. Cl. CPC C12Q 1/6883; A61K 31/519; A61K 39/3955; C07K 16/241; C12Q 2600/16; C12Q 2600/158; C12Q 2600/112; C07K 2317/76; A61K 2039/505

(57) ABSTRACT

This invention refers to the field of human medicine and specifically to the diagnosis/prognosis of the responsiveness to a cytokine targeting drug (Cy TD) or an anti-inflammatory biological drug treatment of a subject suffering from an inflammatory disease. More precisely, the present invention concerns a method for the in vitrodiagnosis/prognosis of a Cy TD or an anti-inflammatory biological drug responsive or non-responsive phenotype, comprising: (a) determining from a subject biological sample an expression profile comprising the gene MKNK1; or of the genes MKNK1 and GNLY; or of the genes MKNK1, TBX21 and TGFBR3; or of the genes MKNK1, GNLY, and ADI1; or of the genes MKNK1, GNLY, ADI1, and IL1B; or of the genes MKNK1, GNLY, ADI1, IL1B, and IL1R1; or of the genes MKNK1, PRF1, TBX21, TGFBR3, IFNGR2, FYN, IL1B and CFLAR; or of the genes MKNK1, PRF1, TBX21, TGFBR3, IFNGR2, FYN, IL1B, CFLAR, MAPK14 and GNLY; or of the genes MKNK1, PRF1, TBX21, TGFBR3, IFNGR2, FYN, IL1B, CFLAR, CD14 and TGFBR2; or of the genes MKNK1, IFNGR2, IL1B, MAPK14, GNLY, and CD14; or of the genes MKNK1, PRF1, TBX21, TGFBR3, IFNGR2, IL1B, CFLAR, MAPK14, GNLY, CD14 and TGFBR2; or of all the 46 genes of following Tables 2, 3 and 4; or Equivalent Expression Profile thereof, provided that, in said Equivalent Expression Profile thereof, MKNK1 is not replaced by gene S 100A8 nor gene MAPK14, and (ii) optionally one or more housekeeping gene(s), (b) comparing the obtained expression profile with at least one reference expression profile, and (c) determining the responsive or non-responsive phenotype from said comparison. The present invention also relates to kits and nucleic acid microarrays for performing said method. The present invention also relates to methods of treatment of inflammatory disease-suffering patients.

### FIELD OF THE INVENTION

The present invention relates to the field of diagnosis or prognosis of a responding or non-responding phenotype to various biological drugs useful for treatment of inflammatory diseases, as well as associated therapeutic uses and methods.

### BACKGROUND ART

Cytokine targeting drugs and more generally anti-inflammatory biological drugs such as TNFα-blocking agents (herein after referred to as “TBA”) are increasingly used in the treatment of various inflammatory diseases. The first indications in which such TBA were approved are rheumatoid arthritis and Crohn's disease. Rheumatoid arthritis (RA) is a chronic, progressive, debilitating auto-immune disease of largely unknown etiology that affects approximately 1% of the population (1). RA is characterized by chronic inflammation of the synovium, which ultimately leads to joint damage, pain and disability (2). The clinical spectrum of RA is heterogeneous, ranging from mild to severe, with variability in secondary organ system involvement. Disease heterogeneity is further illustrated by the current variation in treatment response rates. First line treatment is usually initiated with so called disease-modifying anti-rheumatic drugs (DMARDs), such as methotrexate (MTX). Approximately 30% of patients display a suboptimal response or intolerance to traditional DMARDs (3). In these patients, second line treatment is initiated with “biologics”, agents that block molecules or cells thought to be instrumental to disease progression, such as tumor necrosis factor-α (TNFα) and interleukin-1 (IL-1) or B and T-cells. There are indeed nine biologic agents currently available, each with overlapping or unique mechanisms of action (4). The response rates to such treatments vary widely, with a great number of patients remaining refractory to treatment or demonstrating only partial improvement (5). The incomplete understanding of drug mechanisms of action together with disease heterogeneity means that there are no methods of identifying patient suitability for the various biologics prior to the initiation of the treatment. Establishing a rational basis on which to select patients for specific biologics would help patients to be treated more efficiently; those that would be likely to respond would initiate the biologic in question whereas those unlikely to respond could be provided with another treatment.

In absence of reliable literature on efficacy and safety of biologics and given the percentage of patients that do not respond or experience severe adverse effects, the destructive nature of RA, and the societal costs of inefficacious biological treatments, there is a strong need to make predictions on success before starting the therapy. A clinically or radiographic-based test will most probably assess conditions too late for protecting joints from irreversible destruction. Ideally, a molecular biomarker signature as a predictor for therapy responsiveness should be obtained prior to the start of therapy in a readily available bio-sample, such as peripheral blood. Given the systemic nature of RA and the communication between the systemic and organ-specific compartments, the peripheral blood may not directly have implications for the understanding of disease pathogenesis, but it is especially suitable to analyze gene expression profiles that provide a framework to select clinically relevant biomarkers. Furthermore, blood-based tests remain less invasive for the patients than synovial tissue-based tests.

Ultimately, this may lead to a personalized form of medicine, whereby the best suited therapy will be applied to an individual patient.

The same is true for other inflammatory diseases for which TBA have been approved or for which preliminary results indicate that TBA might be a useful treatment. For the moment, TBA have been approved, in addition to the treatment of RA or Crohn's disease, for the treatment of ankylosing spondylitis, psoriatic arthritis, plaque psoriasis, and ulcerative colitis (see notably FDA labels of infliximab and etanercept). In addition, preliminary results suggest that TBA may be useful in the treatment of several other inflammatory diseases, such as vasculitis (notably Behcet's disease, Churg-Strauss vasculitis, polyarteritis nodosa, and giant cell arthritis); Wegener's granulomatosis; sarcoidosis; adult-onset Still's disease, polymyositis/dermatomyositis, and systemic lupus erythematosus (SLE) (31 and 32).

In all cases, only a proportion (although sometimes a high proportion) of patients treated with TBA display a clinical response to the treatment (see notably FDA labels of infliximab (Remicade®) and etanercept (Enbrel®)). For all diseases in which TBA may be useful, it would thus be very helpful to be able to predict the capacity of a subject to respond or not to TBA treatment.

A very powerful way to gain insight into the molecular signatures underlying pathophysiological processes has arisen from DNA microarray technology, which allows the identification of the fraction of genes that are differentially expressed in blood and tissue samples among patients with clinically defined disease. These differentially expressed genes may provide insight into biological pathways contributing to disease and represent classifiers for early diagnosis, prognosis, and response prediction.

Several pitfalls were experienced using this multistage and relatively expensive technology, which highly depends on perfectly standardized conditions. Factors that might influence the sensitivity and reproducibility range from sample differences, variation in amount and quality of starting RNA material, amplification and labeling strategies and dyes, to probe sequence and hybridization conditions. In addition, the lack of standardized approaches for normalization and usage of data analysis algorithms could influence the outcome. Furthermore, most microarray studies are not prospectively planned and often do not have detailed protocols, but rather tend to make use of existing samples. Therefore, verification of results is an essential step in microarrays studies and quality criteria have to be set.

Several groups have explored the possibility of identifying molecular traits (single nucleotide polymorphisms, gene expression etc.) capable of classifying patients according to their response to treatment based on retrospective analyses of biological samples (synovium or peripheral blood) collected at treatment baseline. In particular, much interest has been paid to the TNFα-blocking agent Infliximab, with the first report several years ago by T Lequerré and colleagues of gene expression-based prediction of response to therapy (6). Since then, several other groups have similarly reported on large-scale gene expression analyses of peripheral blood as a means to predict response to Infliximab (7) (8) (9). All of these studies reported on differentially expressed genes and combinations thereof for the prediction of response to therapy.

These studies provided important proof of concept for the prediction of response to Infliximab at baseline of therapy. Nevertheless, as with all studies of this kind, the use of microarray technology, measuring thousands of genes simultaneously in relatively small cohorts of patients, runs the risk of over-fitting data, leading to false positive results. Moreover, the mono-centric nature of these studies may limit the relevance of the genes identified to a wider and more demographically varied population.

The present invention overcomes these drawbacks by combining information from multiple existing studies. This approach can increase the reliability and generalizability of results. Quantitative approaches in which individual studies addressing a set of related research hypothesis are statistically integrated and analyzed to determine the effectiveness of interventions (meta-analysis) showed the broad utility of applying meta-analytic approaches to genome-wide data for the purpose of biological discovery. Meta-analysis were already used to identify genes differentially expressed between two groups, to compare results obtained on different microarray platforms (cross-platform classification), to identify overlaps between samples from heterologous datasets, to identify co-expressed genes or to reconstruct gene networks.

Meta-analyses of multiple gene expression microarray datasets provide discriminative gene expression signatures that are identified and validated on a large number of microarray samples, generated by different laboratories and microarray technologies. Predictive models generated by this approach are better validated than those generated on a single data set, while showing high predictive power and improved generalization performance.

In the present invention, the meta-analysis was performed according to the stepwise approach in conducting meta-analysis on microarray datasets (1-identify suitable microarray studies; 2-extract data from studies (this step also involved getting additional information from the authors of selected studies); 3-prepare the individual datasets; 4-annotate the individual datasets; 5-resolve the many-to-many relationship between probes and genes; 6-combine the study-specific estimates; 7-analyze, present, and interpret results) described in Ramasamy et al. (10).

Forty six distinct genes differentially expressed between future responders and non responders to Infliximab therapy have thus been identified. Furthermore, one particular gene (MKNK1) has been found to be highly correlated to the Infliximab responsive or non-responsive phenotype of tested subjects, and several combinations of a minimum number of genes are proposed as being predictive of the primary (week 14, week 16 and week 22) response to anti-TNF treatment in RA patients. These combinations comprise genes that are known to be involved in inflammatory or immune processes rather than in the metabolism pathway of Infliximab, which clearly gives a rational for their general usefulness for predicting TNFα-blocking agents (TBA) responsive or non-responsive phenotype of subjects suffering from other inflammatory diseases, notably those for which TBA have been approved or have been shown to be useful in preliminary studies.

### DETAILED DESCRIPTION OF THE INVENTION

The invention thus relates to a method for the in vitro diagnosis or prognosis of a cytokine targeting drug (hereafter referred to as CyTD, such as a TNFα-blocking agent, hereafter referred to as TBA) or anti-inflammatory biological drug responding or non-responding phenotype, comprising:

(a) determining from a biological sample of a subject suffering from an inflammatory disease an expression profile comprising or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s),

(b) comparing the obtained expression profile with at least one reference expression profile, and

(c) determining the CyTD or anti-inflammatory biological drug responding or non-responding phenotype from said comparison.

The diagnosis or prognosis method according to the invention permits to determine whether a subject is non-responding or responding to a cytokine targeting drug or anti-inflammatory biological drug treatment. In a preferred embodiment, the diagnosis or prognosis methods according to the invention are intended for the diagnosis or prognosis of non-response (i.e. of a non-responding condition) to a CyTD (preferably a TBA, notably Infliximab) or anti-inflammatory biological drug (preferably a TBA, notably Infliximab). In this case, the methods according to the invention for the diagnosis or prognosis of a non-responding phenotype have the advantage to present high negative predictive value (NPV) and high specificity.

For such diagnosis or prognosis of a non-responding phenotype, a non-responding condition or test outcome is considered a positive result, while a responding condition or test outcome is considered a negative result. True and false positive results, NPV, PPV, specificity, sensitivity and error rate are defined and calculated as follows:

| | | Condition (non-responding) | |
|---|---|---|---|
| | | Positive | Negative |
| Test outcome | Positive | True Positive (TP) | False positive (FP) |
| (non-responding) | Negative | False negative (FN) | True negative (TN) |

PPV=TP/(TP+FP)

NPV=TN/(TN+FN)

Specificity=TN/(TN+FP)

Sensitivity=TP/(TP+FN)

Error rate=(FP+FN)/Total number of patients

The invention also relates to a method for designing a CyTD or anti-inflammatory biological drug treatment for a subject suffering from an inflammatory disease, said method comprising:

(a) determining from a biological sample of said subject an expression profile comprising or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s),

(b) comparing the obtained expression profile with at least one reference expression profile,

(c) determining the CyTD or anti-inflammatory biological drug responding or non-responding phenotype from said comparison, and

(d) designing the dose of CyTD or anti-inflammatory biological drug treatment according to said identified CyTD or anti-inflammatory biological drug responding or non-responding phenotype.

In the above method for designing a CyTD or anti-inflammatory biological drug treatment for a subject suffering from an inflammatory disease, the dose that is designed and optionally administered to the subject depends on its responding or non-responding phenotype. In particular, a usual dose may be administered if the subject is diagnosed or prognosed as responding. In contrast, if the subject is diagnosed or prognosed as non-responding, it may be decided not to administrate the CyTD or anti-inflammatory biological drug treatment or to increase the dose.

The invention is also drawn to a method of treatment of a subject suffering from an inflammatory disease with a CyTD or anti-inflammatory biological drug, comprising:

(a) determining from a biological sample of the said subject the presence of a CyTD or anti-inflammatory biological drug responding or non-responding phenotype using a method according to the invention, and

(b) adapting the CyTD or anti-inflammatory biological drug treatment in function of the result of step (a).

Said adaptation of the CyTD or anti-inflammatory biological drug treatment may consist in:
a reduction or suppression of the said CyTD or anti-inflammatory biological drug treatment and a switch to an alternative treatment if the subject has been diagnosed or prognosed as CyTD or anti-inflammatory biological drug non-responding respectively, or
the continuation of the said CyTD or anti-inflammatory biological drug treatment if the subject has been diagnosed or prognosed as CyTD or anti-inflammatory biological drug responding respectively.

The invention also refers to a new use of a CyTD or anti-inflammatory biological drug in the treatment of an inflammatory disease, comprising the steps of:

(a) determining from a biological sample of a subject suffering from an inflammatory disease the presence of a CyTD or anti-inflammatory biological drug responding or non-responding phenotype using a method according to the invention, and

(b) determining the dose of CyTD or anti-inflammatory biological drug to administer with respect to the result of step (a).

Optionally; the dose of CyTD or anti-inflammatory biological drug determined in step (b) is administered to the subject.

The invention thus relates to a CyTD or anti-inflammatory biological drug, for use in treating an inflammatory disease, wherein the CyTD or anti-inflammatory biological drug is administered to a subject suffering from said inflammatory disease who has been diagnosed and/or prognosed as responsive using a method according to the invention.

A CyTD or anti-inflammatory biological drug of the present invention is preferably a TNF alpha blocking agent such as defined below.

The invention also relates to the use of a CyTD or anti-inflammatory biological drug for preparing a drug for the treatment of an inflammatory disease in subjects suffering from said inflammatory disease who have been diagnosed and/or prognosed as responsive using a method according to the invention.

In all the present description, the definitions appear in bold characters.

An “inflammatory disease” refers to a disease involving uncontrolled inflammation processes leading to body damages, and includes any disease generally considered as inflammatory diseases by those skilled in the art.

Advantageously, said inflammatory disease is known to involve, at least in some cases, a pathogenic inflammatory cytokine (IL-1, IL-6, IL15, IL-17, IL-18, IL-23 or TNF-α) secretion. The methods according to the invention then permit to diagnose the presence of such a pathogenic inflammatory cytokine secretion in a tested subject, to predict his/her capacity to respond to a CyTD treatment, and thus to adapt his/her treatment in view of his CyTD responding/non-responding phenotype. Even more advantageously, said inflammatory disease is known to involve, at least in some cases, a pathogenic TNF-α secretion. The methods according to the invention then permit to diagnose the presence of such a pathogenic TNF-α secretion in a tested subject, to predict his/her capacity to respond to a TBA treatment, and thus to adapt his/her treatment in view of his TBA responding/non-responding phenotype.

The methods according to the invention also permit to more generally diagnose a sub type of rheumatoid arthritis or a particular state of activation of the immune system in a tested subject, to predict his/her capacity to respond to an anti-inflammatory biological drug treatment, and thus to adapt his/her treatment in view of his anti-inflammatory biological drug responding/non-responding phenotype.

Such inflammatory diseases may be of autoimmune or non-autoimmune origin. Non limiting examples of inflammatory diseases for which the methods and kits according to the invention are useful, in particular for determining a TBA responding or non-responding phenotype, include rheumatoid arthritis (RA), Crohn's disease, ankylosing spondylitis, psoriatic arthritis, plaque psoriasis, ulcerative colitis, vasculitis (notably Behcet's disease, Churg-Strauss vasculitis, polyarteritis nodosa, and giant cell arthritis); Wegener's granulomatosis; sarcoidosis; adult-onset Still's disease, polymyositis/dermatomyositis, and systemic lupus erythematosus (SLE). An advantageous group of diseases for which the methods of the invention are useful are those in the treatment of which TBA have been approved: rheumatoid arthritis (RA), Crohn's disease, ankylosing spondylitis, psoriatic arthritis, plaque psoriasis, ulcerative colitis. The methods according to the invention are particularly useful for RA-suffering patients for determining a TBA responding or non-responding phenotype. They are also useful for RA-suffering patients for determining an anti-inflammatory biological drug (in particular a recombinant protein inhibiting costimulation of T cells by antigen presenting cells) responding or non-responding phenotype.

For rheumatoid arthritis (RA), since first line treatment is usually initiated with so called disease-modifying anti-rheumatic drugs (DMARDs), the invention also refers to a method of treatment of an RA-suffering subject, comprising the steps of:

(a) administering a therapeutic dose of a DMARD to the said subject suffering from RA,

(b) determining from a biological sample of the said RA-suffering subject the presence of a CyTD or anti-inflammatory biological drug responding or non-responding phenotype using a method according to the invention, and

(c) determining the dose of CyTD or anti-inflammatory biological drug to administer with respect to the result of step (b).

Thus the invention also refers to a combination of 1) a DMARD and 2) a CyTD or anti-inflammatory biological drug, for the treatment of RA, comprising the steps of:

(a) administering a therapeutic dose of a DMARD to a subject suffering from RA,

(b) determining from a biological sample of the said RA-suffering subject the presence of a CyTD or anti-inflammatory biological drug responding or non-responding phenotype using a method according to the invention, and

(c) determining the dose of CyTD or anti-inflammatory biological drug to administer with respect to the result of step (b).

Optionnally; the dose of CyTD or anti-inflammatory biological drug determined in step (c) is administered to the subject.

In a preferred embodiment, the DMARD is methotrexate (MTX).

By “cytokine targeting drug” or “CyTD”, it is meant any molecule neutralizing a cytokine signalling, notably by binding to and neutralizing the cytokine or its receptor. Such a binding and neutralizing molecule may notably be an antibody or a fragment thereof specific for said cytokine or cytokine receptor, cytokine receptor antagonists, or any other molecule, such as a recombinant protein, binding to and neutralizing said cytokine or cytokine receptor. Said CyTD preferably targets an inflammatory cytokine such as IL-1, IL-6, IL-15, IL-17, IL-18, IL-23 or TNF-α or a receptor of such inflammatory cytokines. Molecules targeting IL-1 signalling include monoclonal antibodies to IL-1, such as Canakinumab (commercial name Ilaris®), a human anti-IL-1β monoclonal antibody; antagonists of IL-1 receptor such as anakinra (commercial name Kineret®), and a fusion protein between IgG1 Fc portion and ligand-binding domains of human IL-1RI and IL-1AcP such as Rilonacept (nom commercial Arcalyst™) Molecules targeting II-6 signalling notably include Tocilizumab, an anti-IL-6R monoclonal antibody. Molecules targeting II-15 signalling notably include HuMax-IL-15 (AMG 714), an anti-IL-15 monoclonal antibody. Molecules targeting II-17 signalling notably include AIN457, an anti-IL-17A monoclonal antibody. In all the present description, a preferred embodiment of a CyTD is a “TNFα-blocking agent” or “TBA”.

By “TNFα-blocking agent” or “TBA”, it is herein meant a biological agent which is capable of neutralizing the effects of TNFα. Said agent is a preferentially a protein such as a soluble TNFα receptor, e.g. Pegsunercept, or an antibody. In a further preferred embodiment, the said agent is a monoclonal antibody. In an even further preferred embodiment, the said agent is selected in the group consisting of Etanercept (Enbrel®), Infliximab (Remicade®), Adalimumab (Humira®), Certolizumab pegol (Cimzia®), and golimumab (Simponi®). In an even more preferred embodiment, the said agent is Infliximab.

By “anti-inflammatory biological drug”, it is herein meant a biological agent (preferably a recombinant protein, including recombinant antibodies) with anti-inflammatory properties. This includes CyTD directed to inflammatory cytokines such as IL-1, IL-6, IL-15, IL-17, IL-18, IL-23 or TNF-α (in particular TBAs) or to a receptor of such inflammatory cytokines. This also notably includes the following biological agents (preferably recombinant proteins, including recombinant antibodies):
inhibitors of T cell costimulation by antigen presenting cells such as abatacept (Orencia®) or belatacept (Nulojix®), which are fusion proteins composed of an immunoglobulin Fc fragment fused to the extracellular domain of CTLA-4 (a B7 binding molecule), and which interfere with the binding of T cells CD28 molecules to antigen presenting cells B7 molecules;
B cells inhibitors, such as rituximab (Rituxan®), an anti-CD20 monoclonal antibody; and
inhibitors of leukocyte trafficking, such as natalizumab (Tysabri®), and anti-alpha 4-integrin antibody.

In addition to TBAs, and notably infliximab, another preferred anti-inflammatory biological drug is abatacept.

In a particularly preferred embodiment of any method according to the present invention, the inflammatory disease is rheumatoid arthritis and the CyTD or anti-inflammatory biological drug is Infliximab (Remicade®), a particular TBA. In still another preferred embodiment of any method according to the present invention, the inflammatory disease is rheumatoid arthritis and the CyTD or anti-inflammatory biological drug is abatacept (Orencia®).

According to the present invention, a “CyTD or anti-inflammatory biological drug responding phenotype” is defined as a response state of a subject to the administration of a CyTD or anti-inflammatory biological drug respectively. A “response state” means that the said subject (referred to as a CyTD or anti-inflammatory biological drug responding subject or a responding subject or a responsive subject: for the purpose of this application, these terms are similar) responds to the treatment, i.e. that the treatment is efficacious in the said subject. The definition of response is an improvement in clinical symptoms. The quantification of such response is made according to ACR20, ACR50, ACR70 criteria (11) and/or EULAR criteria between 14 and 22 weeks, and notably at weeks 14, 16 or 22 weeks or change in DAS28>1.2. Even more preferred is EULAR response criteria at 14 weeks. These criteria (31) have been established by organizations regrouping the professionals in the field (ACR: American College of Rheumatology; EULAR: European League Against Rheumatism). These criteria are thus well known to the skilled person in the art and need not be detailed here.

In contrast, a “CyTD or anti-inflammatory biological drug non-responding phenotype” refers to the absence in said subject (referred to herein as a CyTD or anti-inflammatory biological drug non-responding subject or a non responding subject or a non-responsive subject: these terms should be construed in the context of this application as having the same meaning) of a state of response, meaning that said subject remains refractory to the treatment.

In a preferred embodiment of any of the above-described in vitro methods of diagnosis/prognosis according to the invention, the said subject is an RA-suffering subject. An “RA-suffering subject” is a subject fulfilling the American College of Rheumatology (ACR) criteria for RA (11). In one further embodiment, the said subject is not treated with a CyTD or anti-inflammatory biological drug; in another further embodiment, the said subject is treated with a CyTD or anti-inflammatory biological drug.

It will easily be conceived that when the said subject is not yet treated with a CyTD or anti-inflammatory biological drug, the methods of the invention permit a prognosis (also referred to as a prediction) of the responsiveness/non responsiveness of the said subject. Thus, in this embodiment, the method of the invention allows the person skilled in the art to prognose (i.e. to identify or predict) the subjects susceptible of responding to the CyTD or anti-inflammatory biological drug treatment. This is important because of the destructive nature of RA and the societal costs of inefficacious biological treatments. Moreover, since this embodiment of the invention allows for identification of non responsive subjects before any treatment is initiated (i.e. prediction of non response), the risks for one treated subject to encounter severe adverse effects are greatly diminished. Moreover, they are useful for predicting subjects who are not responding to the treatment, i.e. who are refractory to the CyTD or anti-inflammatory biological drug, and should thus be administered another therapy. In particular, the methods of the invention allow for a prediction/prognosis of response or non-response at week 14, 16 or 22 from the beginning of the CyTD or anti-inflammatory biological drug treatment.

When the subject according to the invention is already treated with a CyTD or anti-inflammatory biological drug, the methods of the invention are useful for diagnosing if a subject responds to the said CyTD or anti-inflammatory biological drug, and whether the said subject would thus benefit from a continuation of the said treatment. Moreover, they are useful for diagnosing subjects who are not responding to the treatment, i.e. who are refractory to the CyTD or anti-inflammatory biological drug, and should thus be swiftly shifted to another therapy. In regard of the debilitating nature of RA, this achievement is crucial. In particular, the methods of the invention allow for a diagnosis at week 14 or 22 after the beginning of the CyTD or anti-inflammatory biological drug treatment.

In the present description, what is described for CyTD or anti-inflammatory biological drug also particularly applies to TBA, which is a preferred embodiment of a CyTD or anti-inflammatory biological drug in any method or kit according to the invention.

A “biological sample” may be any sample that may be taken from a subject, such as a serum sample, a plasma sample, a urine sample, a blood sample, in particular a peripheral blood sample, a lymph sample, or a biopsy. It also includes specific cellular subtypes or derivatives extracted from those such as PBMCs. Such a sample must allow for the determination of an expression profile comprising or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

Preferred biological samples for the determination of an expression profile include samples such as a blood sample, a plasma sample, a lymph sample, or a biopsy. Preferably, the biological sample is a blood sample. Indeed, such a blood sample may be obtained by a completely harmless blood collection from the patient and thus allows for a non-invasive diagnosis or prognosis of a CyTD or anti-inflammatory biological drug responding or non-responding phenotype.

Optionally, all methods according to the invention may further comprise a preliminary step of taking a biological sample from the patient.

By “gene” is meant the Official Gene Symbol provided by HUGO Gene Nomenclature Committee (www.genenames.orq) and used in the Entrez Gene database, the NCBI's repository for gene-specific information (http://www.ncbi.nlm.nih.qov/qene).

By “expression profile” is meant the expression levels of a group of genes comprising or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s), since these expression profiles have been demonstrated to be particularly relevant for assessing the responding/non responding phenotype of a subject.

In a most preferred embodiment, the expression profile for diagnosing or prognosing (i.e. predicting) if the subject is responding or not at week 14, 16 or week 22 after the beginning of the CyTD or anti-inflammatory biological drug treatment comprises or preferably consists of the gene MKNK1 or Equivalent Expression Profile thereof, provided that, in said Equivalent Expression Profile thereof, MKNK1 is not replaced by gene S100A8 nor gene MAPK14, and optionally one or more housekeeping gene(s). In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1 and GNLY, or Equivalent Expression Profile thereof, provided that, in said Equivalent Expression Profile thereof, MKNK1 is not replaced by gene S100A8 nor gene MAPK14, and optionally one or more housekeeping gene(s).

In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, TBX21 and TGFBR3, or Equivalent Expression Profile thereof, provided that, in said Equivalent Expression Profile thereof, MKNK1 is not replaced by gene S100A8 nor gene MAPK14, and optionally one or more housekeeping gene(s).

In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, GNLY, and ADI1, or Equivalent Expression Profile thereof, provided that, in said Equivalent Expression Profile thereof, MKNK1 is not replaced by gene S100A8 nor gene MAPK14, and optionally one or more housekeeping gene(s).

In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, GNLY, ADI1, and IL1B, or Equivalent Expression Profile thereof, provided that, in said Equivalent Expression Profile thereof, MKNK1 is not replaced by gene S100A8 nor gene MAPK14, and optionally one or more housekeeping gene(s).

In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, GNLY, ADI1, IL1B, and IL1R1, or Equivalent Expression Profile thereof, provided that, in said Equivalent Expression Profile thereof, MKNK1 is not replaced by gene S100A8 nor gene MAPK14, and optionally one or more housekeeping gene(s).

In yet another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, PRF1, TBX21, TGFBR3, IFNGR2, FYN, IL1B and CFLAR, or Equivalent Expression Profile thereof, provided that, in said Equivalent Expression Profile thereof, MKNK1 is not replaced by gene S100A8 nor gene MAPK14, and optionally one or more housekeeping gene(s).

In still another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, PRF1, TBX21, TGFBR3, IFNGR2, FYN, IL1B, CFLAR, MAPK14 and GNLY or Equivalent Expression Profile thereof, provided that, in said Equivalent Expression Profile thereof, MKNK1 is not replaced by gene S100A8 nor gene MAPK14, and optionally one or more housekeeping gene(s).

In yet another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, PRF1, TBX21, TGFBR3, IFNGR2, FYN, IL1B, CFLAR, CD14 and TGFBR2 or Equivalent Expression Profile thereof, provided that, in said Equivalent Expression Profile thereof, MKNK1 is not replaced by gene S100A8 nor gene MAPK14, and optionally one or more housekeeping gene(s).

In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, IFNGR2, IL1B, MAPK14, GNLY, and CD14, or Equivalent Expression Profile thereof, provided that, in said Equivalent Expression Profile thereof, MKNK1 is not replaced by gene S100A8 nor gene MAPK14, and optionally one or more housekeeping gene(s).

In another most preferred embodiment, the expression profile for diagnosing or prognosing responsiveness or non responsiveness at week 14, week 16 or week 22 after the beginning of the CyTD or anti-inflammatory biological drug treatment (in particular a TBA treatment) comprises or preferably consists of the genes MKNK1, PRF1, TBX21, TGFBR3, IFNGR2, IL1B, CFLAR, MAPK14, GNLY, CD14 and TGFBR2, or Equivalent Expression Profile thereof, provided that, in said Equivalent Expression Profile thereof, MKNK1 is not replaced by gene S100A8 nor gene MAPK14, and optionally one or more housekeeping gene(s).

The inventors have determined that MKNK1 is a key gene for determining if a subject suffering from an inflammatory disease, in particular an RA-suffering subject, will or not respond to a CyTD or anti-inflammatory biological drug treatment, in particular a TBA treatment and more particularly an Infliximab treatment. The methods according to the invention are thus mainly based on the determination of an expression profile comprising or consisting of gene MKNK1.

However, the addition of a small number of other genes to the tested expression profile improves the sensitivity, specificity, positive predictive value (PPV) and/or negative predictive value (NPV), and thus decreases the error rate of the diagnosis or prognosis and the determined expression profiles thus preferably comprises at least 1, preferably at least 2, at least 3, at least 4, or at least 5 other genes.

In particular, an expression profile comprising MKNK1 preferably comprises 2, 3, 4, 5, 6, 7, 8, 9, 10, 11, 12, 13, 14, 15, 16, 17, 18, 19, or 20 genes useful for the diagnosis or prognosis, including MKNK1, and optionally further comprises one or more housekeeping gene(s) used for normalization of the data.

An expression profile comprising MKNK1 may notably comprise 2-20 genes useful for the diagnosis or prognosis, and in particular 5-15 genes, 6-12 genes, or 8-10 genes useful for the diagnosis or prognosis, including MKNK1, and may optionally further comprise one or more housekeeping gene(s) useful for normalization of the data.

Such an expression profile comprising MKNK1 may notably be chosen from expression profiles comprising the specific combinations of genes disclosed in the present specification, such as those mentioned above.

The determination of the presence of a CyTD or anti-inflammatory biological drug responding or non-responding phenotype is carried out thanks to the comparison of the obtained expression profile with at least one reference expression profile in step (b).

The term “Equivalent Expression Profile” herein refers to expression profiles comprising or consisting of:
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of following Tables 2, 3 and 4,

wherein the addition, deletion or substitution of some of the genes (preferably at most 1 or 2 genes) does not change significantly the reliability of the test and is considered as an “acceptable expression profile”.

In a preferred embodiment, Equivalent Expression Profiles include expression profiles in which one of the genes of a selected genes combination is replaced by an equivalent gene. In the present description, a first gene (“gene A”) can be considered as equivalent to another second gene (“gene B”), when replacing “gene A” in the expression profile of by “gene B” does not significantly impact the performance of the test. This is typically the case when “gene A” is correlated to “gene B”, meaning that the expression of “gene A” is statistically correlated to the expression level of “gene B”, as determined by a measure such as Pearson's correlation coefficient. The correlation may be positive (meaning that when “gene A” is upregulated in a patient, then “gene” B is also upregulated in that same patient) or negative (meaning that when “gene A” is upregulated in a patient, then “gene B” is downregulated in that same patient). Then fact that replacing “gene A” by a correlated “gene B” still permits reliable diagnosis or prognosis of responding or non-responding phenotype is demonstrated for genes with an average Pearson's correlation coefficient of at least 0.9 in Example 3.

In another embodiment, the addition or substitution of some of the genes of the sets described in the present invention by other genes belonging to the same metabolic pathway should also be considered as an equivalent expression profile.

For instance, genes that are equivalent to MKNK1 or to one of those of the herein disclosed genes combinations and that may be used in addition or as a substitution in an Equivalent Expression Profile include those disclosed in FIGS. 1-2 and in Tables 7-9 below. In Table 7 and Table 8 are listed genes that are the most correlated to each of the key genes, the correlation being calculated by measure of Pearson's correlation coefficient based on two types of pangenomic microarray data (the table therefore lists the top genes amongst thousands of genes that are measured by Illumina and Affymetrix microarray data and should be quite exhaustive). In Table 9 are listed genes that are significantly correlated to each of the key genes, the correlation being calculated by measure of Pearson's correlation coefficient based on qPCR validation experiments performed by the inventors (the correlations were performed on a much shorter list of genes (60 genes) and can be used (since it is qPCR based and unlike data from Table 7 and 8) to exemplify the effect on our assay performance (which is done in Table 10).

Illustrative embodiments of Equivalent expression profiles are disclosed in Example 3. Further illustrative Equivalent expression profiles include expression profiles comprising or consisting of at least 1 gene from at least 6 of the following 10 groups:
Group A, wherein Group A consists of gene MKNK1 and correlated genes as determined in Tables 7 to 9, except S100A8 and MAPK14 (MKNK1; NLRP12; FAM129A; NFKBIZ; KCNJ15; C19orf59; SRPK1; IL1R2; CSF2RA; ACSL1; AQP9; TMEM8; MTMR3; SOD2; CENTD2; NTNG2; DGAT2; PROK2; DYSF; RASSF2; USP32; C15orf28; MAP3K2; QKI; CDC42EP3; NPL; tcag7.907; UIMC1; PELI1; U2AF1; MLX; SAMD4B; REM2; YPEL3; YIPF1; PBX2; NUMB; CKAP4; LOC284454; MAPK1; CD11c, IFNGR2, IFITM2);
Group B, wherein Group B consists of gene PRF1 and correlated genes as determined in Tables 7 to 9 (PRF1; GNLY; IL2RB; GPR114; GZMH; RASSF1; GZMB; HIP1; SH2D2A; CDC42SE1; LMO4; GPR56; FGFBP2; SPON2; TCP1 ACLY; ALKBH5; R3HDM2; KIR3DL1; NKG7; TBXAS1; CTA221G9.4; TRGV9; TARP; TRGC2; GPR68; FGFBP2; CCL5; KIR3DL3///LILRP2///KIR2DL3; CX3CR1; CECR1; RUNX3; TBX21; CCR5; TGFBR3; and CCL4);
Group C, wherein Group C consists of gene TBX21 and correlated genes as determined in Tables 7 to 9 (TBX21; CNTN2; POU5F1; CPEB4; RNF185; CAMK1; PRKD3; MLANA; TMEM161B; LMTK2; AZGP1; GMEB1; HDAC8; GSPT2; KNCN; DPM3; CCDC52; C20orf12; AADAC; CEP70; FLJ22374; CHST12; KLRD1; S1PR5; JAKMIP1; MYO6; SOX13; LOC100133241///OSBPL5; GFI1; TTC38; OSBPL5; GPR56; CTA221G9.4; EOMES; C1orf21; NKG7; GZMH; TRGV9; TARP; TRGC2; CCR5; TGFBR3; CCL4; IL2RB; GNLY; and PRF1);
Group D, wherein Group D consists of gene TGFBR3 and correlated genes as determined in Tables 7 to 9 (TGFBR3; EOMES; C1orf21; GZMH; TRGV9; TARP; TRGC2; GPR68; FGFBP2; NKG7; GZMA; LGR6; XCL2; PPP2R2B; CCR5; HOPX; PYHIN1; PDGFD; KLRK1; SIGLECP3; XCL1; ABCB1; TBX21; CCR5; CCL4; IL2RB; GNLY; ENPP4; FYN; and PRF1);
Group E, wherein Group E consists of gene IFNGR2 and correlated genes as determined in Tables 7 to 9 (IFNGR2; CFLAR; SIRPA; ZFP36L1; ARHGAP9; TM6SF1; HEXB; FAM65B; SLC37A3; SLC26A8; FKBP1A; NUMB; FBXL5; VNN2; FAM49A; HOXD8; ATP6V1A; PIK3CD; ALOX5AP; SRPK1; TLR4; ACSL1; PREX1; NDEL1; VNN1; DHRS7; PTEN; NCLN; RTN3; PANX2; SLC25A44; NADK; REPS2; OAZ2; PISD; TMCC3; BST1; DIRC2; CKAP4; LOC284454; IL8RB; IL6R; IL1RN; MAPK14; HCK00210; TGFBR2; CD16; CD11c; TIMP2; MKNK1; TNFAIP9; CASC3; and IFITM2);
Group F, wherein Group F consists of gene FYN and correlated genes as determined in Tables 7 to 9 (FYN; RPUSD2; ADA; PARP1; CARD11; CD81; NDUFS8; C9orf142; ULK3; WDR54; RUNX3; NUP210; DDX56; FLJ21438; KIAA1310; B3GALT6; SMARCD1; POLG; NOL14; FAM62A; DDOST; DAZAP1; SYNCRIP; GIMAP1; CBLB; EZR; MAPRE2; CBX6; AFF1; HNRNPD; CBX5; UHMK1; PURA; NVL; CALM1; YEATS2; ATP5G3; UCP2; PABPC1; SET; DYRK2; SLC25A3; ELK4; ABCB1; CCR5; TGFBR3; IL2RB; and KLRK1);
Group G, wherein Group G consists of gene IL1B and correlated genes as determined in Tables 7 to 9 (IL1B; ADM; RERE; KCNJ2; AXUD1; RBM23; PCDHGB5; FCGR2A; ACSL1; GADD45B; NAMPT; RNF149; AQP9; CEBPB; TRIM21; LIMK2; TNFSF13B; PIK3AP1; CEACAM1; STX11; GK; LOC100129443_KIAA0040; BCL3; LOC646470; PIM3; GADD45B; PPIF; JUNB; FLJ39051; STAT5B; C16orf57; LIMK2; GLT1D1; MRPL44; DHRS13; ALPL; ADM; IL1RN; and TLR5);
Group H, wherein Group H consists of gene CFLAR and correlated genes as determined in Tables 7 to 9 (CFLAR; CASP9; F11R; METTL2A; SNAP23; RRAGD; MVP; MAFF; SERPINB1; JARID1B; TLR4; HIST1H2BC; OSBPL2; FRAT1; RBP7; CSF2RA; IL1R2; IL13RA1; TTRAP; RPGRIP1; CBL; SSH2; MGEA5; LOC647190; SORL1; FAM126B; FAM120A; SMCHD1; MAP3K3; PHF20L1; SPAG9; APAF1; LOC730051; EIF2C2; MLL2; FNIP1; FYB; DKFZP434B2016; NUMB; IL8RB; MAPK14; HCK002110; TGFBR2; TNFAIP9; IFNGR2; IL1R1; IFITM2; and DECR1);
Group I, wherein Group I consists of gene MAPK14 and correlated genes as determined in Tables 7 to 9 (MAPK14; IL8RB; MMP9; IL6R; IL1RN; HCK002110; CD16; CD11c; SLC2A3; TIMP2; MKNK1; HSPA1A; TNFAIP9; CFLAR; IFNGR2; TLR5; CASC3; and IFITM2); and
Group J, wherein Group J consists of gene GNLY and correlated genes as determined in Tables 7 to 9 (GNLY; TBX21; TGFBR3; CCL4; IL2RB; and PRF1).

As may be observed in FIG. 1 and in Table 7 below, S100A8 can be considered as equivalent to MKNK1. Since expression profiles in which MKNK1 is replaced by gene S100A8 have been disclosed for the same purpose in previous U.S. applications Nos. 61/282,732 and 61/457,158, and in PCT application n° PCT/EP2011/054569, they have been excluded of the scope of the methods of the present invention. As may be observed in Table 9 below, MAPK14 can also be considered as equivalent to MKNK1. Since expression profiles in which MKNK1 is replaced by gene MAPK14 have been disclosed for the same purpose in previous U.S. applications Nos. 61/282,732 and 61/457,158, and in PCT application n° PCT/EP2011/054569, they have also been excluded of the scope of the methods of the present invention.

In all methods, kits or microarrays described herein, when the expression profile comprises or consists of the gene MKNK1; or of the genes MKNK1, TBX21 and TGFBR3; or of the genes MKNK1, PRF1, TBX21, TGFBR3, IFNGR2, FYN, IL1B and CFLAR; or of all the 46 genes of following Tables 2, 3 and 4, it is further possible to exclude Equivalent Expression Profiles that comprise genes MAPK14 and/or S100A8.

In this case, the methods, kits or microarrays according to the invention are based on an expression profile comprising or consisting of the gene MKNK1; or of the genes MKNK1, TBX21 and TGFBR3; or of the genes MKNK1, PRF1, TBX21, TGFBR3, IFNGR2, FYN, IL1B and CFLAR; or of all the 46 genes of following Tables 2, 3 and 4 or Equivalent Expression Profile thereof, provided that said Equivalent Expression Profile thereof does not comprise gene MAPK14 nor gene S100A8.

The term “Acceptable Expression Profile” herein refers to an expression profile which is capable of correctly classifying at least 60% of the analyzed samples, preferably 65%, and more preferably 70%, and even more preferably 75%, 80% or 85%; and has a sensitivity and specificity of at least 60% preferably 65%, and more preferably 70%, and even more preferably 75 or 80%.The sensitivity value is defined as the ratio of the number of patients actually clinically responding to the CyTD or anti-inflammatory biological drug treatment and classified as responding using the test according to the invention amongst all patients treated with the CyTD or anti-inflammatory biological drug. Specificity measures the proportion of patients actually clinically not responding to the CyTD or anti-inflammatory biological drug treatment which are correctly identified using the test according to the invention amongst all patients treated with the CyTD or anti-inflammatory biological drug.

By “Best Expression Profile” is meant an expression profile which is able to correctly classify at least 80% of the analyzed samples, has either a sensitivity or a sensitivity of at least 80%.

Although the lists of the gene MKNK1; or of the genes MKNK1 and GNLY; or of the genes MKNK1, TBX21 and TGFBR3; or of the genes MKNK1, GNLY, and ADI1; or of the genes MKNK1, GNLY, ADI1, and IL1B; or of the genes MKNK1, GNLY, ADI1, IL1B, and IL1R1; or of the genes MKNK1, PRF1, TBX21, TGFBR3, IFNGR2, FYN, IL1B and CFLAR; or of the genes MKNK1, PRF1, TBX21, TGFBR3, IFNGR2, FYN, IL1B, CFLAR, MAPK14 and GNLY; or of the genes MKNK1, PRF1, TBX21, TGFBR3, IFNGR2, FYN, IL1B, CFLAR, CD14 and TGFBR2; or of the genes MKNK1, IFNGR2, IL1B, MAPK14, GNLY, and CD14; or of the genes MKNK1, PRF1, TBX21, TGFBR3, IFNGR2, FYN, IL1B, CFLAR, MAPK14, GNLY, CD14 and TGFBR2; or of the genes MKNK1, IFNGR2, IL1B, MAPK14, GNLY, and CD14; or of the genes MKNK1, PRF1, TBX21, TGFBR3, IFNGR2, IL1B, CFLAR, MAPK14, GNLY, CD14 and TGFBR2; or of all the 46 genes of following Tables 2, 3 and 4 have been determined as the Best Expression Profiles to assess responsiveness/non responsiveness, an Equivalent Expression Profile such as defined above, still permits to assess responsiveness, with an acceptable reliability. In particular embodiments, sublists of the gene MKNK1; or of the genes MKNK1 and GNLY; or of the genes MKNK1, TBX21 and TGFBR3; or of the genes MKNK1, GNLY, and ADI1; or of the genes MKNK1, GNLY, ADI1, and IL1B; or of the genes MKNK1, GNLY, ADI1, IL1B, and IL1R1; or of the genes MKNK1, PRF1, TBX21, TGFBR3, IFNGR2, FYN, IL1B and CFLAR; or of the genes MKNK1, PRF1, TBX21, TGFBR3, IFNGR2, FYN, IL1B, CFLAR, MAPK14 and GNLY; or of the genes MKNK1, PRF1, TBX21, TGFBR3, IFNGR2, FYN, IL1B, CFLAR, CD14 and TGFBR2; or of the genes MKNK1, IFNGR2, IL1B, MAPK14, GNLY, and CD14; or of the genes MKNK1, PRF1, TBX21, TGFBR3, IFNGR2, FYN, IL1B, CFLAR, MAPK14, GNLY, CD14 and TGFBR2; or of the genes MKNK1, IFNGR2, IL1B, MAPK14, GNLY, and CD14; or of the genes MKNK1, PRF1, TBX21, TGFBR3, IFNGR2, IL1B, CFLAR, MAPK14, GNLY, CD14 and TGFBR2; or of all the 46 genes of following Tables 2, 3 and 4; still permit to assess responsiveness with a good reliability and should be considered as Acceptable Expression Profiles.

While the expression profile used for determining the CyTD (notably TBA) or anti-inflammatory biological drug responsive or non-responsive phenotype may comprise and not only consist of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, FYN, IL1B, CFLAR, MAPK14, GNLY, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s),
   it is preferred that the expression profiles consists or essentially consists of one of the above described (i) expression profiles, optionally with one or more housekeeping gene(s), meaning that no more than 50, 40, 30, 25, 20, preferably no more than 15, preferably no more than 10, preferably no more than 9, 8, 7, 6, 5, 4, 3, 2, or 1 genes that are not a gene belonging to one the above described (i) expression profiles or a housekeeping gene are included in the expression profile.

By “housekeeping genes”, it is meant genes that are constitutively expressed at a relatively constant level across many or all known conditions, because they code for proteins that are constantly required by the cell, hence, they are essential to a cell and always present under any conditions. It is assumed that their expression is unaffected by experimental conditions. The proteins they code are generally involved in the basic functions necessary for the sustenance or maintenance of the cell. Non-limitating examples of housekeeping genes that may be used in methods of the invention include:
HPRT1 (hypoxanthine phosphoribosyltransferase 1),
UBC (ubiquitin C),
YWHAZ (tyrosine 3-monooxygenase/tryptophan 5-monooxygenase activation protein, zeta polypeptide),
B2M (beta-2-microglobulin),
GAPDH (glyceraldehyde-3-phosphate dehydrogenase),
FPGS (folylpolyglutamate synthase),
DECR1 (2,4-dienoyl CoA reductase 1, mitochondrial),
PPIB (peptidylprolyl isomerase B (cyclophilin B)),
ACTB (actin 13),
PSMB2 (proteasome (prosome, macropain) subunit, beta type, 2),
GPS1 (G protein pathway suppressor 1),
CANX (calnexin),
NACA (nascent polypeptide-associated complex alpha subunit),
TAX1BP1 (Taxi (human T-cell leukemia virus type I) binding protein 1), and
PSMD2 (proteasome (prosome, macropain) 26S subunit, non-ATPase, 2).

When such housekeeping genes are added to the expression profile (it is not always necessary), they are used for normalization purpose. In this case, the number of housekeeping genes used for normalization in methods according to the invention is preferably comprised between one and five with a preference for three.

The determination of the presence of a responsive or non responsive phenotype is carried out thanks to the obtained expression profile with at least one reference profile in step (b).

A “reference expression profile” is a predetermined expression profile, obtained from a biological sample from a subject with a known particular response state. In particular embodiments, the reference expression profile used for comparison with the test sample in step (b) may have been obtained from a biological sample from a CyTD or anti-inflammatory biological drug responsive subject (“CyTD or anti-inflammatory biological drug responsive reference expression profile” or “responsive reference expression profile”; as used herein these expressions are synonymous), and/or from a biological sample from a CyTD or anti-inflammatory biological drug non-responsive subject (“CyTD or anti-inflammatory biological drug non-responsive reference expression profile” or “non-responsive reference expression profile”; as used herein these expressions have the same meaning).

Preferably, at least one reference expression profile is a CyTD or anti-inflammatory biological drug responsive reference expression profile. Alternatively, at least one reference expression profile may be a CyTD or anti-inflammatory biological drug non-responsive reference expression profile. More preferably, the determination of the presence or absence of a CyTD or anti-inflammatory biological drug responsive phenotype is carried out by comparison with at least one responder and at least one non-responder reference expression profiles. The diagnosis or prognostic may thus be performed using one responsive reference expression profile and one non-responsive reference expression profile. Advantageously, to get a stronger diagnosis or prognostic, said diagnosis or prognostic is carried out using several responsive reference expression profiles and several non-responsive reference expression profiles.

The comparison of a tested subject expression profile with said reference expression profiles, which permits prediction of the tested subject's clinical response based on his/her expression profile, can be done by those skilled in the art using statistical models or machine learning technologies. The PLS (Partial Least Square) regression is particularly relevant to give prediction in the case of small reference samples. The comparison may also be performed using Support Vector Machines (SVM), linear regression or derivatives thereof (such as the generalized linear model abbreviated as GLM, including logistic regression), Linear Discriminant Analysis (LDA), Random Forests, k-NN (Nearest Neighbour) or PAM (Predictive Analysis of Microarrays) statistical methods. More precisely, a group of reference samples, which is generally referred to as training data, is used to select an optimal statistical algorithm that best separates responders from non responders (like a decision rule). The best separation is usually the one that misclassifies as few samples as possible and that has the best chance to perform comparably well on a different dataset.

Typically for a binary outcome such as responder/non-responder, this is done using a generalized linear model abbreviated as GLM, including logistic regression. Logistic regression is based on the determination of a logistic regression function

$$f ( z ) = \frac{{e}^{z}}{{e}^{z} + 1} = \frac{1}{1 + {e}^{- z}} ,$$

in which z is usually defined as z=β₀+β₁x₁+ . . . +βₙxₙ, wherein x₁ to xₙ are the expression values of the n genes in the signature, β₀ is the intercept, and β₁ to βₙ are the regression coefficients. The values of the intercept and of the regression coefficients are determined based on a group of reference samples (“training data”). f(z) then defines the probability that a test expression profile is responding or non-responding (when defining f(z) based on training data, the user decides if the probability is a probability of response or of non-response). A test expression profile is then classified as responding or non-responding depending if the probability that it is responding or non-responding is inferior or superior to a particular threshold value, which is also determined based on training data. Sometimes, two threshold values are used, defining an undetermined area. Other types of generalized linear models than logistic regression may also be used.

Alternative methods such as nearest neighbour (abbreviated as k-NN) are also commonly used and predict response or non-response for a new sample based on whether the sample is closer to the group of responders or to the group of non-responders. The notion of “closer” is based on a choice of distance (metric, such as but not limited to Euclidian distance) in the n-dimension space defined by a signature consisting of n genes useful for diagnosis or prognosis (thus excluding potential housekeeping genes used for normalization purpose). The distances between a test expression profile and all reference responding or non-responding expression profiles are calculated and the sample is classified by analysis of the k closest reference samples (k being an positive integer of at least 1 and most commonly 3 or 5), a rule of classification being pre-established depending of the number of responding or non-responding reference expression profiles among the k closest reference expression profiles. For instance, when k is 1, a test expression profile is classified as responding if the closest reference expression profile is a responding expression profile, and as non-responding if the closest reference expression profile is a non-responding expression profile. When k is 2, a test expression profile is classified as responding if the two closest reference expression profiles are responding expression profiles, as non-responding if the two closest reference expression profiles are non-responding expression profiles, and undetermined if the two closest reference expression profiles include a responding and a non-responding reference expression profile. When k is 3, a test expression profile is classified as responding if at least two of the three closest reference expression profiles are responding expression profiles, and as non-responding if at least two of the three closest reference expression profiles are non-responding expression profiles. More generally, when k is p, a test expression profile is classified as responding if more than half of the p closest reference expression profiles are responding expression profiles, and as non-responding if more than half of the p closest reference expression profiles are non-responding expression profiles. If the numbers of responding and non-responding reference expression profiles are equal, then the test expression profile is classified as undetermined.

Other methodologies from the field of statistics, mathematics or engineering exist, for example but not limited to decision trees, Support Vector Machines (SVM), Neural Networks and Linear Discriminant Analyses (LDA). These approaches are well known to people skilled in the art.

In summary, an algorithm (which may be selected from linear regression or derivatives thereof such as generalized linear models (GLM, including logistic regression), nearest neighbour (k-NN), decision trees, support vector machines (SVM), neural networks, linear discriminant analyses (LDA), Random forests, or Predictive Analysis of Microarrays (PAM) is calibrated based on a group of reference samples (preferably including several responsive reference expression profiles and several non-responsive reference expression profiles) and then applied to the test sample. In simple terms, a patient will be classified as responder (or non-responder) based on how all the genes in the signature compare to all the genes from a reference profile that was developed from a group of responders (training data).

The notion of whether individual genes of the expression profile are increased or decreased in a responder versus a non-responders is of scientific interest. For each individual gene, the gene expression levels in the responder group can be compared to the non-responder group by the use of Student's t-test or equivalent methods. However, such binary comparisons are generally not used for diagnosis or prognosis when a signature comprises several distinct genes.

The expression profile may be determined by any technology known by a man skilled in the art. In particular, each gene expression level may be measured at the genomic and/or nucleic and/or proteic level. In a preferred embodiment, the expression profile is determined by measuring the amount of nucleic acid transcripts of each gene. In another embodiment, the expression profile is determined by measuring the amount of protein produced by each of the genes.

The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-know in the art. From the mRNA or cDNA sample, the amount of nucleic acid transcripts may be measured using any technology known by a man skilled in the art, including nucleic microarrays, quantitative PCR, next generation sequencing and hybridization with a labelled probe.

In a preferred embodiment, the expression profile is determined using quantitative PCR. Quantitative, or real-time, PCR is a well known and easily available technology for those skilled in the art and does not need a precise description.

In a particular embodiment, which should not be considered as limiting the scope of the invention, the determination of the expression profile using quantitative PCR may be performed as follows. Briefly, the real-time PCR reactions are carried out using the TaqMan Universal PCR Master Mix (Applied Biosystems). 6 μL cDNA is added to a 9 μL PCR mixture containing 7.5 μL TaqMan Universal PCR Master Mix, 0.75 μL of a 20× mixture of probe and primers and 0.75 μl water. The reaction consisted of one initiating step of 2 min at 50 deg. C, followed by 10 min at 95 deg. C, and 40 cycles of amplification including 15 sec at 95 deg. C and 1 min at 60 deg. C. The reaction and data acquisition can be performed using the ABI 7900HT Fast Real-Time PCR System (Applied Biosystems). The number of template transcript molecules in a sample is determined by recording the amplification cycle in the exponential phase (cycle threshold or C_{Q} or C_{T}), at which time the fluorescence signal can be detected above background fluorescence. Thus, the starting number of template transcript molecules is inversely related to C_{T}. The level of expression of a gene is measured using the “ΔΔCT method”, briefly a gene is normalized by the value of one or a group of reference/housekeeping genes and/or by a reference sample such as a pooled sample or a commercially available reference such as the qPCR Human Universal Reference cDNA, random primed; Ozyme; réf 639654.

In another preferred embodiment, the expression profile is determined by the use of a nucleic microarray.

According to the invention, a “nucleic microarray” consists of different nucleic acid probes that are attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes can be nucleic acids such as cDNAs (“cDNA microarray”) or oligonucleotides (“oligonucleotide microarray”), and the oligonucleotides may be about 25 to about 60 base pairs or less in length.

To determine the expression profile of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art.

In a preferred embodiment, the nucleic microarray is an oligonucleotide microarray comprising or consisting of oligonucleotides specific for:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

Preferably, the oligonucleotides are about 50 bases in length. It is acknowledged that the nucleic acid microarray or oligonucleotide microarray of the invention encompasses the microarrays specific for an Equivalent Expression Profile as defined above.

Suitable microarray oligonucleotides specific for any gene of Tables 2, 3 and 4 may be designed, based on the genomic sequence of each gene (see Tables 2, 3 and 4 Genbank accession numbers), using any method of microarray oligonucleotide design known in the art. In particular, any available software developed for the design of microarray oligonucleotides may be used, such as, for instance, the OligoArray software (available at http://berry.enqin.umich.edu/oliqoarray/), the GoArrays software (available at http://www.isima.fr/bioinfo/goarrays/), the Array Designer software (available at http://www.premierbiosoft.com/dnamicroarray/index.html), the Primer3 software (available at http://frodo.wi.mit.edu/primer3/primer3 code.html), or the Promide software (available at http://oligos.molgen.mpg.de/).

In another embodiment, the expression profile is determined by the use of a protein microarray.

In a particular embodiment of a method according to the invention, said method may further comprise determining at least one additional parameter useful for the diagnosis or prognosis. Such “parameters useful for the diagnosis or prognosis” are parameters that cannot be used alone for a diagnosis or prognosis but that have been described as displaying significantly different values between responsive subjects and subjects who are clearly refractory and may thus also be used to refine and/or confirm the diagnosis or prognosis according to the above described method according to the invention. They may notably include relevant clinical parameters depending on the inflammatory disease. For rheumatoid arthritis (RA), such clinical parameters include an assessment of the subject's pain, duration of morning stiffness, the number of swollen joints, the number of painful joints etc. Preferably, the parameters useful for diagnosis or prognosis are determined from a non invasive biological sample of the subject. In particular, for RA, they may be selected from standard biological parameters specific for RA. According to the invention, “standard biological parameters specific for RA” are biological parameters usually used by clinicians to monitor the efficacy of a treatment of RA. These standard biological parameters specific for RA or autoimmune diseases usually comprise serum or plasma concentrations of particular proteins which are well known of those skilled in the art. The said standard biological parameters specific for RA can be determined by tests which include the Antinuclear Antibody test (ANA test), C-Reactive Protein test (CRP test), Erythrocyte sedimentation rate (ESR test), Cyclic Citrullinated Peptide Antibody test (CCP test), and the Rheumatoid Factor test. These tests are well known to the person skilled in the art and not be detailed here. They may be used on their own or in combination.

Such additional parameters may be used to confirm the diagnosis or prognosis obtained using the expression profile comprising or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

The invention further concerns a kit for the in vitro diagnosis or prognosis of a CyTD or anti-inflammatory biological drug responsive or non responsive phenotype, comprising at least one reagent for the determination of an expression profile comprising, or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, does MKNK1 is not replaced by gene S100A8 nor gene MAPK14, and
(ii) optionally one or more housekeeping gene(s).

By “a reagent for the determination of an expression profile” is meant a reagent which specifically allows for the determination of said expression profile, i.e. a reagent specifically intended for the specific determination of the expression level of the genes comprised in the expression profile. This definition excludes generic reagents useful for the determination of the expression level of any gene, such as Taq polymerase or an amplification buffer, although such reagents may also be included in a kit according to the invention.

In a preferred embodiment of a kit according to the invention, said kit is dedicated to the in vitro diagnosis or prognosis of a CyTD or anti-inflammatory biological drug responsive or non responsive phenotype based on expression profiles comprising or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or Equivalent Expression Profile thereof, provided that, in said Equivalent Expression Profile thereof, MKNK1 is not replaced by gene S100A8 nor gene MAPK14, and
(ii) optionally one or more housekeeping gene(s).

By “dedicated”, it is meant that reagents for the determination of an expression profile in the kit of the invention essentially consist of reagents for determining the expression level of the above (i) expression profiles, optionally with one or more housekeeping gene(s), and thus comprise a minimum of reagents for determining the expression of other genes than those mentioned in above described (i) expression profiles and housekeeping genes. For instance, a dedicated kit of the invention preferably comprises no more than 50, 40, 30, 25, 20, preferably no more than 15, preferably no more than 10, preferably no more than 9, 8, 7, 6, 5, 4, 3, 2, or 1 reagent(s) for determining the expression level of a gene that does not belong to one of the above described (i) expression profiles and that is not a housekeeping gene.

Such a kit for the in vitro diagnosis or prognosis of a CyTD or anti-inflammatory biological drug responsive or non responsive phenotype may further comprise instructions for determination of the presence or absence of a responsive phenotype.

Such a kit for the in vitro diagnosis or prognosis of a responsive phenotype may also further comprise at least one reagent for the determining of at least one additional parameter useful for the diagnosis or prognosis such as standard biological parameters. In particular, the said reagent is useful for performing any of the following tests: the Antinuclear Antibody test (ANA test), C-Reactive Protein test (CRP test), Erythrocyte sedimentation rate (ESR test), Cyclic Citrullinated Peptide Antibody test (CCP test), and the Rheumatoid Factor test.

In any kit for the in vitro diagnosis or prognosis of a responsive phenotype according to the invention, the reagent(s) for the determination of an expression profile comprising, or consisting of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s),
   preferably include specific amplification primers and/or probes for the specific quantitative amplification of transcripts of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s), and/or
   a nucleic microarray for the detection of:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

The determination of the expression profile may thus be performed using quantitative PCR and/or a nucleic microarray, preferably an oligonucleotide microarray, and/or protein microarrays.

In addition, the instructions for the determination of the presence or absence of a CyTD (notably TBA) or anti-inflammatory biological drug phenotype preferably include at least one reference expression profile, or at least one reference sample for obtaining a reference expression profile. In a preferred embodiment, at least one reference expression profile is a responsive expression profile. Alternatively, at least one reference expression profile may be a non responsive expression profile. More preferably, the determination of the level of responsiveness is carried out by comparison with both responsive and non-responsive expression profiles as described above.

The invention is also directed to a nucleic acid microarray comprising or consisting of nucleic acids specific for:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

Said nucleic acid microarray may comprise additional nucleic acids specific for additional genes and optionally one or more housekeeping gene(s), but preferably consists of a maximum of 500, 400, 300, 200 preferably 100, 90, 80, 70 more preferably 60, 50, 45, 40, 35, 30, 25, 20, 15, 10, or even less (for instance 9, 8, 7, 6, 5, 4, 3, 2 or 1) distinct nucleic acids.

In a preferred embodiment, said nucleic acid microarray comprises no more than 50, 40, 30, 25, 20, preferably no more than 15, preferably no more than 10, preferably no more than 9, 8, 7, 6, 5, 4, 3, 2, or 1 distinct nucleic acids specific for a gene that does not belong to one of the above described (i) expression profiles and that is not a housekeeping gene.

Advantageously, said microarray consists of nucleic acids specific for:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

In a preferred embodiment, said nucleic acid microarray is an oligonucleotide microarray comprising or consisting of oligonucleotides specific for:
(i)
   the gene MKNK1; or
   the genes MKNK1 and GNLY; or
   the genes MKNK1, TBX21 and TGFBR3; or
   the genes MKNK1, GNLY, and ADI1; or
   the genes MKNK1, GNLY, ADI1, and IL1B; or
   the genes MKNK1, GNLY, ADI1, IL1B, and IL1R1; or
   the genes MKNK1, PRF1, TBX21, TGFBR3, IFNGR2, FYN, IL1B and CFLAR; or
   the genes MKNK1, PRF1, TBX21, TGFBR3, IFNGR2, FYN, IL1B, CFLAR, MAPK14 and GNLY; or
   the genes MKNK1, PRF1, TBX21, TGFBR3, IFNGR2, FYN, IL1B, CFLAR, CD14 and TGFBR2; or
   the genes MKNK1, IFNGR2, IL1B, MAPK14, GNLY, and CD14; or
   the genes MKNK1, PRF1, TBX21, TGFBR3, IFNGR2, IL1B, CFLAR, MAPK14, GNLY, CD14 and TGFBR2; or
   all the 46 genes of following Tables 2, 3 and 4; or
   Equivalent Expression Profile thereof, provided that, in said Equivalent Expression Profile thereof, MKNK1 is not replaced by gene S100A8 nor gene MAPK14, and
(ii) optionally one or more housekeeping gene(s).

The present invention also relates to systems (and computer readable medium for causing computer systems) to perform a method of diagnosis or prognosis of a CyTD or anti-inflammatory biological drug responding or non-responding phenotype, based on above described expression profiles.

In particular, in a specific embodiment, the invention also relates to a system 1 (see FIG. 3) for analyzing a biological sample comprising:
(a) a determination module 2 configured to receive a biological sample and to determine expression level information concerning:
   (i)
      the gene MKNK1; or
      the genes MKNK1 and GNLY; or
      the genes MKNK1, TBX21 and TGFBR3; or
      the genes MKNK1, GNLY, and ADI1; or
      the genes MKNK1, GNLY, ADI1, and IL1B; or
      the genes MKNK1, GNLY, ADI1, IL1B, and IL1R1; or
      the genes MKNK1, PRF1, TBX21, TGFBR3, IFNGR2, FYN, IL1B and CFLAR; or
      the genes MKNK1, PRF1, TBX21, TGFBR3, IFNGR2, FYN, IL1B, CFLAR, MAPK14 and GNLY; or
      the genes MKNK1, PRF1, TBX21, TGFBR3, IFNGR2, FYN, IL1B, CFLAR, CD14 and TGFBR2; or
      the genes MKNK1, IFNGR2, IL1B, MAPK14, GNLY, and CD14; or
      the genes MKNK1, PRF1, TBX21, TGFBR3, IFNGR2, IL1B, CFLAR, MAPK14, GNLY, CD14 and TGFBR2; or
      all the 46 genes of Tables 2, 3 and 4; or
      Equivalent Expression Profile thereof, provided that, in said Equivalent Expression Profile thereof, MKNK1 is not replaced by gene S100A8 nor gene MAPK14, and
   (ii) optionally one or more housekeeping gene(s)
(b) a storage device 3 configured to store the expression level information from the determination module;
(c) a comparison module 4 adapted to compare the expression level information stored on the storage device with reference data, and to provide a comparison result, wherein the comparison result is indicative of a responding or non-responding phenotype; and
(d) a display module 5 for displaying a content based in part on the comparison result for the user, wherein the content is a signal indicative of a responding or non-responding phenotype.

In another specific embodiment, the invention further relates to a computer readable medium having computer readable instructions recorded thereon to define software modules including a comparison module and a display module for implementing a method on a computer, said method comprising:
a) comparing with a comparison module 4 an expression level information stored on a storage device 3 with reference data to provide a comparison result, wherein the comparison result is indicative of a responding or non-responding phenotype; and
b) displaying with a display module 5 a content based in part on the comparison result for the user, wherein the content is indicative of a responding or non-responding phenotype.

Embodiments of the invention relating to systems and computer-readable media have been described through functional modules, which are defined by computer executable instructions recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable medium can be any available tangible media that can be accessed by a computer. Computer readable medium includes volatile and nonvolatile, removable and non-removable tangible media implemented in any method or technology for storage of information such as computer readable instructions, data structures, program modules or other data. Computer readable medium includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media, may define instructions, for example, as part of one or more programs, that, as a result of being executed by a computer, instruct the computer to perform one or more of the functions described herein (e.g., in relation to system 1, or computer readable medium), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of 1, or computer readable medium described herein, may be distributed across one or more of such components, and may be in transition there between.

The computer-readable media may be transportable such that the instructions stored thereon can be loaded onto any computer resource to implement the aspects of the present invention discussed herein. In addition, it should be appreciated that the instructions stored on the computer readable media, or the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997, ref 38); Salzberg, Searles, Kasif, (Ed.), Computational Methods in Molecular Biology, (Elsevier, Amsterdam, 1998, ref 39); Rashidi and Buehler, Bioinformatics Basics: Application in Biological Science and Medicine (CRC Press, London, 2000, ref 40) and Ouelette and Bzevanis Bioinformatics: A Practical Guide for Analysis of Gene and Proteins (Wiley & Sons, Inc., 2^{nd} ed., 2001, ref 41).

The functional modules of certain embodiments of the invention include a determination module 2, a storage device 3, a comparison module 4 and a display module 5. The functional modules can be executed on one, or multiple, computers, or by using one, or multiple, computer networks. The determination module 2 has computer executable instructions to provide expression level information in computer readable form.

As used herein, “expression level information” refers to information about expression level of any nucleotide (RNA or DNA) and/or amino acid sequences, either full-length or partial. In a preferred embodiment, it refers to the level of expression of mRNA or cDNA, measured by various technologies. The information may be qualitative (presence or absence of a transcript) or quantitative. Preferably it is quantitative. Methods for determining expression level information, i.e. determination modules 2, include systems for protein and DNA/RNA analysis, and in particular those described above for determination of expression profiles at the nucleic or protein level.

The sequence information determined in the determination module can be read by the storage device 3. As used herein the “storage device” 3 is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices 3 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 3 is adapted or configured for having recorded thereon expression level information. Such information may be provided in digital form that can be transmitted and read electronically, e.g., via the Internet, on diskette, via USB (universal serial bus) or via any other suitable mode of communication including wireless communication between devices.

As used herein, “stored” refers to a process for encoding information on the storage device 3. Those skilled in the art can readily adopt any of the presently known methods for recording information on known media to generate manufactures comprising the expression level information.

A variety of software programs and formats can be used to store the expression level information on the storage device. Any number of data processor structuring formats (e.g., text file, spreadsheets or database) can be employed to obtain or create a medium having recorded thereon the expression level information.

By providing expression level information in computer-readable form, one can use the expression level information in readable form in the comparison module 4 to compare a specific expression profile with the reference data within the storage device 3. The comparison may notably be done using the various algorithms described above. The comparison made in computer-readable form provides a computer readable comparison result which can be processed by a variety of means. Content based on the comparison result can be retrieved from the comparison module 4 and displayed by the display module 5 to indicate a responding or non-responding phenotype.

Preferably, the reference data are expression level profiles that are indicative of both responding and non-responding phenotypes.

The “comparison module” 4 can use a variety of available software programs and formats for the comparison operative to compare expression level information determined in the determination module 2 to reference data, either directly, or indirectly using any software providing statistical classification algorithms such as those already described above.

The comparison module 4, or any other module of the invention, may include an operating system (e.g., Windows, Linux, Mac OS or UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware--as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module 4 provides computer readable comparison result that can be processed in computer readable form by predefined criteria, or criteria defined by a user, to provide a content 6 based in part on the comparison result that may be stored and output as requested by a user using a display module 5. The display module 5 enables display of a content 6 based in part on the comparison result for the user, wherein the content is a signal indicative of a responding or non-responding phenotype. Such signal can be, for example, a display of content indicative of a responding or non-responding phenotype on a computer monitor, a printed page or printed report of content indicating a responding or non-responding phenotype from a printer, or a light or sound indicative of a responding or non-responding phenotype.

The content 6 based on the comparison result varies depending on the algorithm used for comparison.

For instance, when linear regression or derivatives thereof is used, the content 6 may include a probability of being responding or non-responding, or both a probability of being responding or non-responding and one or more threshold values, or merely a signal indicative of a responding or non-responding phenotype. When nearest neighbor (k-NN) is used, the content 6 may include the number or proportion of responding and non-responding expression profiles among the k closest profiles, or merely a signal indicative of a responding or non-responding phenotype. Moreover, the content 6 may simply be a continuous or categorical score reported in a numerical, text or graphical way (for example using a color code such as red, orange or green).

The display module 5 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or from ARM Holdings, or any other type of processor, visual display devices such as flat panel displays, cathode ray tubes and the like, as well as computer printers of various types or integrated devices such as laptops or tablets, in particular iPads.

In one embodiment, a World Wide Web browser is used for providing a user interface for display of the content 6 based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information.

In one embodiment, the display module 5 displays the comparison result and whether the comparison result is indicative of a responding or non-responding phenotype.

In one embodiment, the content 6 based on the comparison result that is displayed is a signal (e.g. positive or negative signal) indicative of a responding or non-responding phenotype, thus only a positive or negative indication may be displayed.

The present invention therefore provides for systems 1 (and computer readable medium for causing computer systems) to perform methods for diagnosing or prognosing a responding or non-responding phenotype, based on expression profiles information.

System 1, and computer readable medium, are merely illustrative embodiments of the invention for performing methods of diagnosing or prognosing a responding or non-responding phenotype based on expression profiles, and is not intended to limit the scope of the invention. Variations of system 1, and computer readable medium, are possible and are intended to fall within the scope of the invention.

The modules of the system 1 or used in the computer readable medium, may assume numerous configurations. For example, function may be provided on a single machine or distributed over multiple machines.

Having generally described this invention, a further understanding of characteristics and advantages of the invention can be obtained by reference to certain specific examples and figures which are provided herein for purposes of illustration only and are not intended to be limiting unless otherwise specified.

### DESCRIPTION OF THE FIGURES

FIG. 1: Networks represent the genes that correlate the most to each of the target genes in the data of Julia et al. (8). Any correlated gene can be considered as equivalent.

FIG. 2: Networks represent the genes that correlate the most to each of the target genes in the data of Bienkowska et al. (9). Any correlated gene can be considered as equivalent.

FIG. 3: Specific, non-limiting, illustration of a system 1 according to the invention, comprising a determination module 2, a storage device 3, a comparison module 4, and a display module 5.

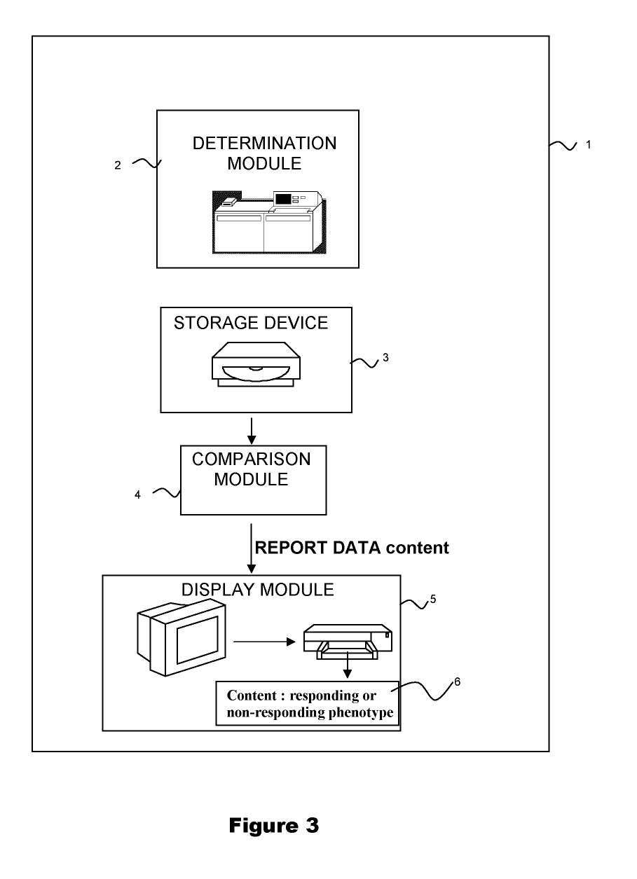

FIG. 4: Plot illustrating the effect of replacing genes by equivalent ones based on different levels of correlation. Based on the original 8 gene model presented in table 10, we replaced each gene by a “virtual” gene which was simulated based on the fact that it correlated to the original gene. This figure illustrates that in this eight gene signature, all genes can be replaced by genes that on average correlate with a coefficient of 0.9 and the prediction still achieves a sensitivity above 60%.

FIG. 5: prediction of non response to Infliximab using Equation 1 as defined in Example 5. All patients above the horizontal line at zero are clinical responders to infliximab and predicted as such by a positive score, all non responders are below the line and predicted as such (negative score).

### EXAMPLES

### Example 1

### Identification of Candidate Genes by Meta-Analysis of Microarray Data

Materials and Methods

In this example, the materials and methodologies used in the subsequent examples are described.

Data Identification and Data Extraction: Studies were selected on the basis that they had been performed on RA patients naive to biologics who had started therapy with Infliximab and measurement of their response to treatment was available at 14 or 22 weeks. Large scale gene expression information had to be available at baseline (prior to treatment). Following the steps described in Ramasamy et al. (10), we identified four six studies that matched our research criteria: Lequerré et al. (6), Sekiguchi et al. (7), Bienkowska et al. (9), and Julià et al. (8), Tanino et al. (36) and van Baarsen et al. (37). The expression data, the phenotypes and the annotation data were all downloaded from GEO (GSE3592, GSE8350, GSE12051 and, GSE15258, GSE20690 and GSE19821 respectively).

All six studies identified “Gene expression signatures of response to anti-TNF therapy”. Interestingly, however, no two publications used the same approach and this can partly explain the lack of overlap between the reported signatures. To make the six studies more comparable, we contacted the authors to obtain additional individual information such as the DAS28 at baseline and week 14, week 16 or week 22 to use a single definition of response (EULAR criteria—with “moderate” and “good” responders considered as responders) or detail of treatment to ensure that only Infliximab-treated patients were analyzed. We therefore reclassified patients as responders based on the EULAR definition at week 14, week 16 and week 22 and performed a binary analysis of good and moderate responders versus non-responders. This binary grouping is particularly suited for the identification of non responders. The final dataset is summarized in Table 1 and was the most homogeneous data we could obtain.

TABLE 1
Details of the different studies used in the meta analysis. Studies are named
according to the first author. Missing information is represented by <<NA>> for Not Available.

| Authors | | Bienkowska | Julià | Lequerre | Sekiguchi | Tanino | van Baarsen |
|---|---|---|---|---|---|---|---|
| Study | PubMed ID | 19699293 | 19847310 | 16817978 | 18388148 | 19577537 | 20096109 |
| Information | GEO accession number | GSE15258 | GSE12051 | GSE3592 | GSE8350 | GSE20690 | GSE19821 |
| | Tissue/Blood | whole blood | whole blood | PBMCs | whole blood | whole blood | whole blood |
| | Microarray | Affymetrix | Illumina | Custom | Custom | Agilent | Custom |
| | Treatment | Infliximab | Infliximab (bi- | Infliximab (bi- | Infliximab (bi- | Infliximab (bi- | Infliximab (bi- |
| | | (mono- | therapy) | therapy) | therapy) | therapy) | therapy) |
| | | therapy) | | | | | |
| | Studied response criteria | EULAR @ | EULAR @ | EULAR @ | EULAR @ | EULAR @ | EULAR @ |
| | | 14 wks | 14 wks | 14 wks | 22 wks | 14 wks | 16 wks |
| | .CEL | Yes | No | No | No | No | No |
| # Sample | Reponder (G&M) | 20 | 37 | 9 | 15 | 28 | 10 |
| | Non-Reponder(N) | 8 | 7 | 4 | 3 | 8 | 5 |
| | Number of patients | 28 | 44 | 13 | 18 | 36 | 15 |
| Array and | Total Probes | 38551 | 15936 | 8356 | 759 | 31537 | 26294 |
| Annotation | Unique Genes with the | 20164 | 15431 | 6404 | 718 | 19328 | 14432 |
| information | highest spot value | | | | | | |
| | Unique Genes with spot | 13952 | 13288 | — | — | 13952 | — |
| | the nearest of the primer | | | | | | |
| Patient | Mean Age (R/NR) | NA | 52/57 | 54.56/56.75 | 55.2/53 | 55.89/51.00 | NA |
| Information | Mean RA duration | NA | 13/19 | 12.78/9.75 | 9.30/4.83 | NA | NA |
| | (R/NR | | | | | | |
| | Mean DAS28 at baseline | 5.36/5.00 | 5.99/5.76 | 6.05/5.97 | 6.19/6.24 | NA | NA |
| | (R/NR) | | | | | | |
| | Mean DAS28 after | 3.28/5.42 | 3.78/5.44 | 4.18/5.78 | 3.67/5.98 | NA | NA |
| | treatment (R/NR) | | | | | | |

Data Quality and Processing: Data from Bienkowska et al. was the only data for which we downloaded the raw .CEL files and processed them using our internal protocols (normalization was performed using GC-RMA in refiner array by GENEDATA Expressionist® (Genedata AG, Basel, Switzerland)). Six chips were flagged with quality issues due to increased distortion. We thus inexcluded them in our analysis.

Data from Lequerré et al., Julia et al. and Sekiguchi et al. were all downloaded as expression matrices, which correspond to expression values after normalization. The data by Lequerré et al. included technical replicates; we averaged the technical replicates and excluded the control samples from the analysis. Following our internal quality control procedures based on the expression profile of sex specific genes, led us to exclude this dataset from further analysis.

To translate probe information into gene information we reblasted the available probe sequences to the latest version of the human genome (Hsap37). Probes were then selected when they mapped within a transcribed region of a gene. When multiple probes were available, we selected the probes that was the closest to the 3′ end of the gene. An alternative approach that was also implemented was to select the probe with the highest average value. Therefore for each gene, only one probe contributed to the analysis.

Statistical Analysis: Because of the impact on the results of using different parameters when performing a meta analysis, we decided to perform three: The first one is based on the most 3′ for probe selection and the Z statistic for variable selection, the second one is based on the most highly expressed probe and the Z statistic for variable selection and the third one is based on the most 3′ for probe selection and the Meta Array package.

The statistical analysis was performed in R using the MetaArray package (34) or a classical Z test. The MetaArray package implements the latent variable model described in (35) as well as the integrative correlation (12). Individual probes contribution was estimated using the t-test as implemented in the multtest library in R. We set the threshold for significance at a p-value of 0.10 because only 2 genes were significant at 5. For the Z test, a p-value of 5% was used as a threshold of significance.

Results

Table 2 provides the Z score value (indicative of the direction), the p-value, the gene symbol, the gene title and the Gene ID (Notation from Entrez Gene NCBI database, updated on December 2010) for the list of significant genes (p-value <5%) based on the 3′ approach to gene mapping and the Z statistic.

TABLE 2
List of significant genes (p-value <5%) based on the
3′ approach to gene mapping and the Z statistic.

| | | Gene | | |
|---|---|---|---|---|
| Z-score | P-value | Symbol | Gene Title | Gene ID |
| 3.39459 | 0.00069 | RHOB | ras homolog gene family, member B | 388 |
| −3.11193 | 0.00186 | PSMA2 | proteasome (prosome, macropain) subunit, alpha | 5683 |
| | | | type, 2 | |
| −2.62277 | 0.00872 | CSE1L | CSE1 chromosome segregation 1-like (yeast) | 1434 |
| 2.59875 | 0.00936 | CSK | c-src tyrosine kinase | 1445 |
| 2.55024 | 0.01076 | MAP2K4 | mitogen-activated protein kinase kinase 4 | 6416 |
| 2.44369 | 0.01454 | IL1B | interleukin 1, beta | 3553 |
| 2.37255 | 0.01767 | YWHAZ | tyrosine 3-monooxygenase/tryptophan 5- | 7534 |
| | | | monooxygenase activation protein, zeta | |
| | | | polypeptide | |
| 2.36878 | 0.01785 | CCL4 | chemokine (C-C motif) ligand 4 | 6351 |
| 2.34906 | 0.01882 | IGF2R | insulin-like growth factor 2 receptor | 3482 |
| 2.31643 | 0.02053 | PRF1 | perform 1 (pore forming protein) | 5551 |
| 2.31634 | 0.02054 | TGFBR3 | Transforming growth factor, beta receptor III | 7049 |
| −2.28062 | 0.02257 | AP1S2 | adaptor-related protein complex 1, sigma 2 | 8905 |
| | | | subunit | |
| 2.26557 | 0.02348 | ITGAL | integrin, alpha L (antigen CD11A (p180), | 3683 |
| | | | lymphocyte function-associated antigen 1 alpha | |
| | | | polypeptide) | |
| 2.24496 | 0.02477 | ADAR | adenosine deaminase, RNA-specific | 103 |
| −2.20781 | 0.02726 | RPA3 | replication protein A3, 14 kDa | 6119 |
| 2.16438 | 0.03044 | IFNGR2 | interferon gamma receptor 2 (interferon gamma | 3460 |
| | | | transducer 1) | |
| −2.10815 | 0.03502 | NDUFV2 | NADH dehydrogenase (ubiquinone) flavoprotein | 4729 |
| | | | 2, 24 kDa | |
| 2.09749 | 0.03595 | SLC11A1 | solute carrier family 11 (proton-coupled divalent | 6556 |
| | | | metal ion transporters), member 1 | |
| 2.09105 | 0.03652 | TIMP2 | TIMP metallopeptidase inhibitor 2 | 7077 |
| 2.08302 | 0.03725 | CP | ceruloplasmin (ferroxidase) | 1356 |
| 2.07606 | 0.03789 | TBX21 | T-box 21 | 30009 |
| 2.02259 | 0.04312 | CCR5 | chemokine (C-C motif) receptor 5 C-C | 1234 |
| | | | chemokine receptor type 5-like | |
| 2.00034 | 0.04546 | TGFB2 | CDNA FLJ11812 fis, clone HEMBA1006364 | |
| 1.99736 | 0.04579 | MKNK1 | MAP kinase interacting serine/threonine kinase 1 | 8569 |

Table 3 provides the Z score value (indicative of the direction), the p-value, the Gene Symbol, the Gene Title and the Gene ID (Notation from Entrez Gene NCBI database, updated on December 2010) based on the most highly expressed probe and the Z statistic.

TABLE 3
Discriminatory performance of the gene set based on
the most highly expressed probe and the Z statistic.

| Z-score | P-value | Gene Symbol | Gene Title | Gene ID |
|---|---|---|---|---|
| 3.088444 | 0.002012 | HSPA1A | heat shock 70 kDa protein 1A | 3303 |
| 3.053196 | 0.002264 | RHOB | ras homolog gene family, | 388 |
| | | | member B | |
| 2.790099 | 0.005269 | ELK4 | ELK4, ETS-domain protein | 2005 |
| | | | (SRF accessory protein 1) | |
| −2.661722 | 0.007774 | HPRT1 | Hypoxanthine | 3251 |
| | | | phosphoribosyltransferase 1 | |
| | | | (Lesch-Nyhan syndrome) | |
| 2.634720 | 0.008421 | CSK | c-src tyrosine kinase | 1445 |
| 2.550244 | 0.010765 | MAP2K4 | mitogen-activated protein | 6416 |
| | | | kinase kinase 4 | |
| 2.478105 | 0.013208 | CP | ceruloplasmin (ferroxidase) | 1356 |
| 2.460926 | 0.013858 | IL1B | interleukin 1, beta | 3553 |
| 2.368784 | 0.017847 | CCL4 | chemokine (C-C motif) | 6351 |
| | | | ligand 4 | |
| 2.356203 | 0.018463 | TGFBR3 | transforming growth factor, | 7049 |
| | | | beta receptor III | |
| 2.349061 | 0.018821 | IGF2R | insulin-like growth factor 2 | 3482 |
| | | | receptor | |
| −2.311675 | 0.020796 | PFDN4 | prefoldin subunit 4 | 5203 |
| 2.276939 | 0.022790 | MKNK1 | MAP kinase interacting | 8569 |
| | | | serine/threonine kinase 1 | |
| 2.270925 | 0.023152 | SLC11A1 | Solute carrier family 11 | 6556 |
| | | | (proton-coupled divalent | |
| | | | metal ion transporters), | |
| | | | member 1 | |
| −2.259800 | 0.023834 | NDUFA4 | NADH dehydrogenase | 4697 |
| | | | (ubiquinone) 1 alpha | |
| | | | subcomplex, 4, 9 kDa | |
| 2.214500 | 0.026794 | IFNGR2 | interferon gamma receptor 2 | 3460 |
| | | | (interferon gamma | |
| | | | transducer 1) | |
| 2.178084 | 0.029400 | CFLAR | CASP8 and FADD-like | 8837 |
| | | | apoptosis regulator | |
| −2.164143 | 0.030453 | HSP90AA1 | heat shock protein 90 kDa | 3320 |
| | | | alpha (cytosolic), class A | |
| | | | member 1 | |
| −2.162666 | 0.030567 | PSMA2 | proteasome (prosome, | 5683 |
| | | | macropain) subunit, alpha | |
| | | | type, 2 | |
| −2.123562 | 0.033707 | COX6A1 | cytochrome c oxidase | 1337 |
| | | | subunit VIa polypeptide 1 | |
| 2.112754 | 0.034622 | ABCB1 | ATP-binding cassette, sub- | 5243 |
| | | | family B (MDR/TAP), | |
| | | | member 1 | |
| −2.108153 | 0.035018 | NDUFV2 | NADH dehydrogenase | 4729 |
| | | | (ubiquinone) flavoprotein 2, | |
| | | | 24 kDa | |
| −2.083767 | 0.037181 | CKS2 | CDC28 protein kinase | 1164 |
| | | | regulatory subunit 2 | |
| 2.076061 | 0.037888 | TBX21 | T-box 21 | 30009 |
| −2.011073 | 0.044318 | NME1 | non-metastatic cells 1, | 4830 |
| | | | protein (NM23A) expressed | |
| | | | in | |
| 2.010690 | 0.044358 | PIAS3 | protein inhibitor of activated | 10401 |
| | | | STAT, 3 | |
| 2.009776 | 0.044455 | ARID3A | AT rich interactive domain | 1820 |
| | | | 3A (BRIGHT-like) | |
| −1.989802 | 0.046613 | RPA3 | replication protein A3, 14 kDa | 6119 |
| 1.972567 | 0.048545 | PLSCR1 | phospholipid scramblase 1 | 5359 |
| 1.972072 | 0.048601 | PML | promyelocytic leukemia | 5371 |

Table 4 provides the Gene Symbol, the Gene Title and the Gene ID (Notation from Entrez Gene NCBI database, updated on December 2010) for the list of borderline significant genes (p-value <10%) based on the 3′ approach to gene mapping and the MetaArray package in R.

TABLE 4
List of borderline significant genes (p-value <10%) based on the
3′ approach to gene mapping and the MetaArray package in R.
List of genes for MetaArray (Genedata + BJTVS)

| Gene | | Gene |
|---|---|---|
| Symbol | Gene Title | ID |
| CASC3 | cancer susceptibility candidate 3 | 22794 |
| NDUFV2 | NADH dehydrogenase (ubiquinone) flavoprotein | 4729 |
| | 2, 24 kDa | |
| ITGA5 | integrin, alpha 5 (fibronectin receptor, alpha | 3678 |
| | polypeptide) | |
| NMI | N-myc (and STAT) interactor | 9111 |
| FYN | FYN oncogene related to SRC, FGR, YES | 2534 |
| HMOX1 | heme oxygenase (decycling) 1 | 3162 |
| CCT6A | chaperonin containing TCP1, subunit 6A (zeta 1) | 908 |

### Example 2

### Validation of Candidate Genes and Signatures

For the 45 genes identified in Example 1, probes specific to Taqman assays were ordered from APPLIED BIOSYSTEMS based on APPLIED's inventoried probes or were designed internally using standard software. After our internal quality control steps 35 out of the 45 genes of the present invention were tested on 40 RA samples. The 40 samples used for the qCPR represent a subset of the original samples used for microarray analysis in the study of Julia et al. The delatadeltaCT method was used to measure gene expression levels after carefully selecting reference genes internally. The following statistical analyses have been performed: Identification of individually differentially expressed genes between the two groups of responders versus non responders (Table 5). The selection criteria used was a significant t-test is p-value<0.05. The following 8 genes were found to be significant: MKNK1, PRF1, TBX21, TGFBR3, IFNGR2, FYN, IL1B and CFLAR.

TABLE 5
P-values from test of responders versus non responders.

| Gene Symbol | Applied Probe | P-value |
|---|---|---|
| MKNK1 | h_MKNK1_Hs00374376_m1_FAM | 0.00288633 |
| PRF1 | h_PRF1_Hs00169473_m1_FAM | 0.00442224 |
| TBX21 | h_TBX21_Hs00894392_m1_FAM | 0.00624481 |
| TGFBR3 | h_TGFBR3_Hs01114253_m1_FAM | 0.00929707 |
| IFNGR2 | h_IFNGR2_Hs00194264_m1_FAM | 0.01069053 |
| FYN | h_FYN_Hs00941600_m1_FAM | 0.01219544 |
| IL1B | h_IL1B_Hs01555410_m1_FAM | 0.02595403 |
| CFLAR | h_CFLAR_Hs01116280_m1_FAM | 0.04107801 |
| CASC3 | h_CASC3_Hs00201226_m1_FAM | 0.05008698 |
| CCT6A | h_CCT6A_Hs00798979_s1_FAM | 0.0574514 |
| HSPA1A | h_HSPA1A_Hs00359163_s1_FAM | 0.06025312 |
| CCL4 | h_CCL4_Hs99999148_m1_FAM | 0.06442333 |
| TIMP2 | h_TIMP2_Hs00234278_m1_FAM | 0.07542514 |
| ACBB1 | h_ABCB1_Hs00184500_m1_FAM | 0.07930045 |
| CSE1L | h_CSE1L_Hs00354853_m1_FAM | 0.08825819 |
| ELK4 | h_ELK4_Hs00360812_m1_FAM | 0.10804976 |
| ADAR | h_ADAR_Hs00241666_m1_FAM | 0.11162474 |
| CCR5 | h_CCR5_Hs99999149_s1_FAM | 0.12023433 |
| NDUFV2 | h_NDUFV2_Hs00221478_m1_FAM | 0.13730798 |
| HPRT1 | h_HPRT1_Hs99999909_m1_FAM | 0.20940829 |
| YWHAZ | h_YWHAZ_Hs00852925_sH_FAM | 0.21031416 |
| PLSCR1 | h_PLSCR1_Hs00275514_m1_FAM | 0.21118584 |
| HSP90AA1 | h_HSP90AA1_Hs00743767_sH_FAM | 0.24323062 |
| COX6A1 | h_COX6A1_Hs01924685_g1_FAM | 0.2639524 |
| PSMA2 | h_PSMA2_Hs00746751_s1_FAM | 0.26592111 |
| AP1S2 | h_AP1S2_Hs00705223_s1_FAM | 0.30722181 |
| PIAS3 | h_PIAS3_Hs00180666_m1_FAM | 0.3256013 |
| CKS2 | h_CKS2_Hs01048812_g1_FAM | 0.3483249 |
| HMOX1 | h_HMOX1_Hs01110250_m1_FAM | 0.4343992 |
| PML | h_PML_Hs00971694_m1_FAM | 0.48460568 |
| ITGA5 | h_ITGA5_Hs01547673_m1_FAM | 0.53697884 |
| MAP2K4 | h_MAP2K4_Hs00387426_m1_FAM | 0.67638462 |
| ITGAL | h_ITGAL_Hs00158218_m1_FAM | 0.7189843 |
| CSK | h_CSK_Hs01062585_m1_FAM | 0.86646143 |
| RHOB | h_RHOB_Hs00269660_s1_FAM | 0.91603175 |

Additionally logistic regression and kNN classification (k=2, 3 and 5) were performed on the eight significant genes to increase the discriminatory performance compared to individual gene discrimination (Table 6). The three gene combination of MKNK1, TBX21 and TGFBR3 was found to be particularly discriminant. The best two gene combination identified from logistic regression using a forward variable selection is MKNK1 and TBX21.

TABLE 6
Results from the various multivariate models tested on subcombinations of
the eight genes. MCC: Matthew's correlation coefficient. AUC: Area Under the Curve.

| | Cross | | | | | | |
|---|---|---|---|---|---|---|---|
| | Validation | Classification | | | Error | | |
| Gene List | Method | Algorithm | Sensitivity | Specificity | Rate | MCC | AUC |
| MKNK1, | Leave One | Logistic | 33.333 | 83.871 | 24.324 | 0.162 | 0.306 |
| PRF1, | Out | Regression | | | | | |
| TBX21, | | | | | | | |
| TGFBR3, | | | | | | | |
| IFNGR2, | | | | | | | |
| FYN, | | | | | | | |
| IL1B and | | | | | | | |
| CFLAR | | | | | | | |
| MKNK1, | ⅓ test set | 2-NN | 100 | 80 | 16.66667 | 0.6324555 | 0.9 |
| PRF1, | | | | | | | |
| TBX21, | | | | | | | |
| TGFBR3, | | | | | | | |
| IFNGR2, | | | | | | | |
| FYN, | | | | | | | |
| IL1B and | | | | | | | |
| CFLAR | | | | | | | |
| MKNK1, | ⅓ test set | 3-NN | 100 | 80 | 16.66667 | 0.6324555 | 0.9 |
| PRF1, | | | | | | | |
| TBX21, | | | | | | | |
| TGFBR3, | | | | | | | |
| IFNGR2, | | | | | | | |
| FYN, | | | | | | | |
| IL1B and | | | | | | | |
| CFLAR | | | | | | | |
| MKNK1, | ⅓ test set | 5-NN | 100 | 100 | 0 | 1 | 1 |
| PRF1, | | | | | | | |
| TBX21, | | | | | | | |
| TGFBR3, | | | | | | | |
| IFNGR2, | | | | | | | |
| FYN, | | | | | | | |
| IL1B and | | | | | | | |
| CFLAR | | | | | | | |
| MKNK1, | Leave One | Logistic | 66.667 | 90.323 | 13.514 | 0.536 | 0.634 |
| TBX21 and | Out | Regression | | | | | |
| TGFBR3 | | | | | | | |
| MKNK1, | ⅓ test set | 2-NN | 100 | 80 | 16.66667 | 0.6324555 | 0.9 |
| TBX21 and | | | | | | | |
| TGFBR3 | | | | | | | |
| MKNK1, | ⅓ test set | 3-NN | 100 | 70 | 25 | 0.5291503 | 0.85 |
| TBX21 and | | | | | | | |
| TGFBR3 | | | | | | | |
| MKNK1, | ⅓ test set | 5-NN | 50 | 80 | 25 | 0.2581989 | 0.45 |
| TBX21 and | | | | | | | |
| TGFBR3 | | | | | | | |
| MKNK1 | Leave One | Logistic | 50.000 | 90.323 | 16.216 | 0.403 | 0.476 |
| and TBX21 | Out | Regression | | | | | |
| MKNK1 | ⅓ test set | 2-NN | 100 | 80 | 16.66667 | 0.6324555 | 0.9 |
| and TBX21 | | | | | | | |
| MKNK1 | ⅓ test set | 3-NN | 100 | 70 | 25 | 0.5291503 | 0.85 |
| and TBX21 | | | | | | | |
| MKNK1 | ⅓ test set | 5-NN | 100 | 70 | 25 | 0.5291503 | 0.85 |
| and TBX21 | | | | | | | |

### Example 3

### Genes Equivalent to the Candidate 8* Genes

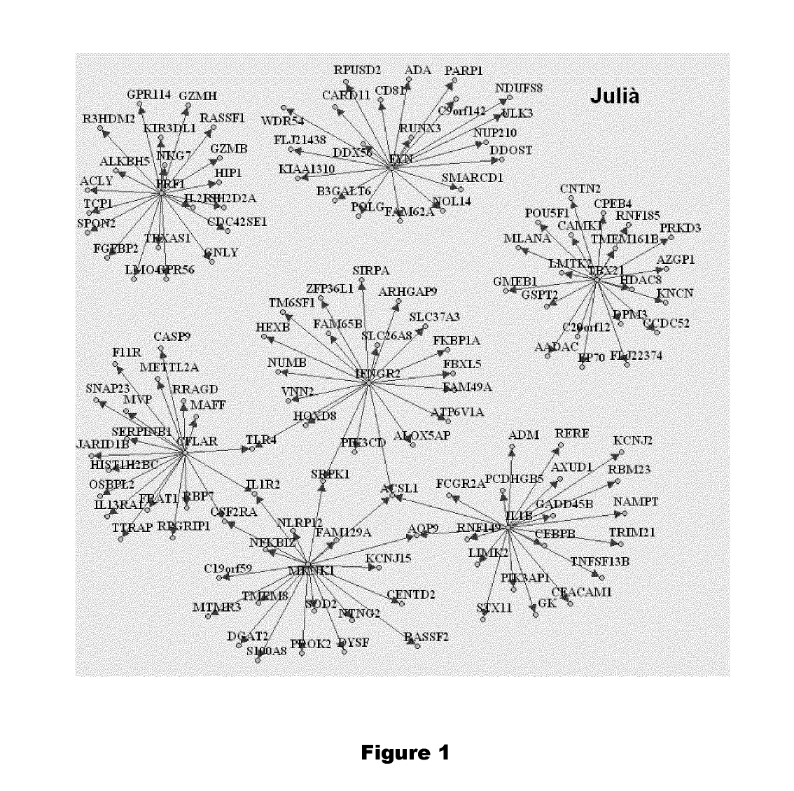

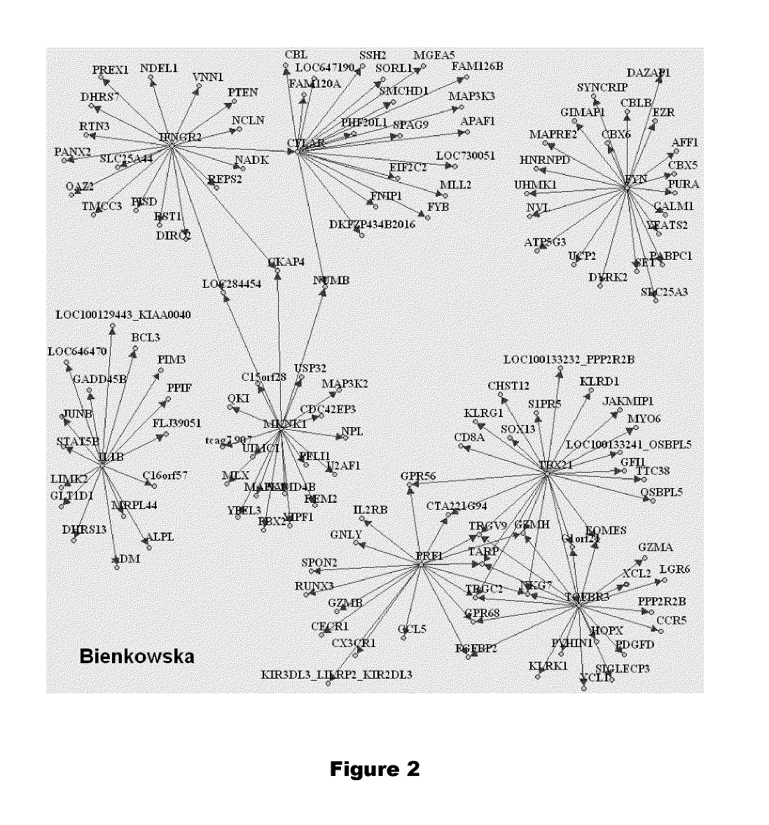

Genes rarely operate individually and thus genes whose expression correlate to other genes can easily be replaced to achieve similar discriminatory performances. To that effect we identified the genes that most correlate to the eight genes from our claim. Correlation analysis is first based on two genomewide datasets to ensure that most genes of the genome are evaluated: the 44 microarray chips of Julia et al. as well as the entire set of microarray chips of Bienkowska et al. (86 chips including other anti-TNFs). The 20 most positively correlated to each of the eight genes are displayed on FIGS. 1 and 2 respectively. They are also indicated in following Tables 7 to 8.

TABLE 7
List of genes that positively correlate to 7 candidate genes based on the data of Julia et al.

| MKNK1 | PRF1 | TBX21 | IFNGR2 | FYN | IL1B | CFLAR |
|---|---|---|---|---|---|---|
| SOD2 | GZMB | HDAC8 | ACSL1 | DDX56 | ADM | CASP9 |
| TMEM8 | GPR56 | CEP70 | HEXB | FLJ21438 | CEBPB | RBP7 |
| AQP9 | ALKBH5 | TMEM161B | TM6SF1 | CD81 | AXUD1 | SNAP23 |
| ACSL1 | R3HDM2 | CPEB4 | FAM49A | NDUFS8 | GADD45B | MVP |
| NFKBIZ | RASSF1 | DPM3 | SLC37A3 | C9orf142 | TRIM21 | JARID1B |
| NLRP12 | SH2D2A | CCDC52 | ARHGAP9 | DDOST | GK | TLR4 |
| SRPK1 | SPON2 | KNCN | FAM65B | ULK3 | LIMK2 | RRAGD |
| FAM129A | HIP1 | POU5F1 | ALOX5AP | NUP210 | KCNJ2 | SERPINB1 |
| NTNG2 | KIR3DL1 | PRKD3 | ZFP36L1 | NOL14 | RNF149 | HIST1H2BC |
| DYSF | GPR114 | LMTK2 | SRPK1 | ADA | PIK3AP1 | |
| CENTD2 | GNLY | FLJ22374 | FKBP1A | WDR54 | FCGR2A | RPGRIP1 |
| S100A8 | NKG7 | AADAC | VNN2 | FAM62A | ACSL1 | OSBPL2 |
| MTMR3 | TBXAS1 | GSPT2 | SIRPA | POLG | CEACAM1 | F11R |
| PROK2 | FGFBP2 | CNTN2 | ATP6V1A | PARP1 | RERE | MAFF |
| KCNJ15 | ACLY | CAMK1 | PIK3CD | RUNX3 | STX11 | TTRAP |
| RASSF2 | GZMH | C20orf12 | TLR4 | B3GALT6 | RBM23 | IL13RA1 |
| DGAT2 | LMO4 | MLANA | NUMB | KIAA1310 | PCDHGB5 | CSF2RA |
| CSF2RA | TCP1 | AZGP1 | SLC26A8 | RPUSD2 | NAMPT | IL1R2 |
| C19orf59 | IL2RB | RNF185 | FBXL5 | CARD11 | AQP9 | FRAT1 |
| IL1R2 | CDC42SE1 | GMEB1 | HOXD8 | SMARCD1 | TNFSF13B | METTL2A |

TABLE 8
List of genes that positively correlate to 8 candidate genes based on the data of Bienkowska et al.

| MKNK1 | PRF1 | TBX21 | IFNGR2 | FYN | IL1B | CFLAR | TGFBR3 |
|---|---|---|---|---|---|---|---|
| NPL | GPR56 | EOMES | RTN3 | YEATS2 | LIMK2 | SMCHD1 | C1orf21 |
| tcag7.907 | SPON2 | SOX13 | PISD | PABPC1 | ADM | PHF20L1 | XCL2 |
| MAP3K2 | NKG7 | C1orf21 | LOC284454 | CALM1 | LIMK2 | SPAG9 | TRGV9 |
| CDC42E | GNLY | TTC38 | NCLN | GIMAP1 | DHRS13 | MGEA5 | LGR6 |
| P3 | | | | | | | |
| REM2 | PRF1 | KLRD1 | PTEN | CBX6 | FLJ39051 | FAM126B | TARP |
| SAMD4B | CTA- | GZMH | OAZ2 | HNRNPD | PPIF | LOC730051 | GPR68 |
| | 221G9.4 | | | | | | |
| YPEL3 | KIR3DL3 | SOX13 | NDEL1 | PURA | GADD45B | FYB | TRGC2 |
| | /// | | | | | | |
| | LILRP2 | | | | | | |
| | /// | | | | | | |
| | KIR2DL3 | | | | | | |
| NUMB | FGFBP2 | TARP | BST1 | SLC25A3 | GADD45B | PHF20L1 | C1orf21 |
| C15orf28 | IL2RB | NKG7 | CKAP4 | MAPRE2 | FLJ39051 | CBL | SIGLECP3 |
| UIMC1 | CCL5 | LOC1001 | REPS2 | SET | GLT1D1 | SSH2 | PYHIN1 |
| | | 33241/// | | | | | |
| | | OSBPL5 | | | | | |
| U2AF1 | TRGV9 | MYO6 | DIRC2 | SYNCRIP | LOC1001 | APAF1 | TARP |
| | | | | | 29443 /// | | |
| | | | | | KIAA0040 | | |
| YIPF1 | RUNX3 | TARP | PREX1 | UCP2 | C16orf57 | EIF2C2 | KLRK1 |
| MAPK1 | TARP | GFI1 | NADK | NVL | STAT5B | NUMB | HOPX |
| QKI | GZMH | GPR56 | CFLAR | DAZAP1 | BCL3 | FAM120A | PPP2R2B |
| PBX2 | GPR68 | CTA- | PANX2 | UHMK1 | ALPL | SORL1 | FGFBP2 |
| | | 221G9.4 | | | | | |
| LOC284454 | CX3CR1 | TRGV9 | TMCC3 | CBLB | MRPL44 | LOC647190 | PDGFD |
| CKAP4 | TARP | CHST12 | SLC25A44 | DYRK2 | GADD45B | FNIP1 | GZMA |
| PELI1 | CECR1 | OSBPL5 | VNN1 | ATP5G3 | JUNB | MLL2 | NKG7 |
| USP32 | CCL5 | TRGC2 | DHRS7 | CBX5 | LOC646470 | MAP3K3 | EOMES |
| MLX | TRGC2 | S1PR5 | SLC25A44 | AFF1 | PIM3 | DKFZP4 | GZMH |
| | | | | | | 34B2016 | |
| | GZMB | C1orf21 | CFLAR | EZR | | | XCL1 |
| | | JAKMIP1 | | | | | CCR5 |

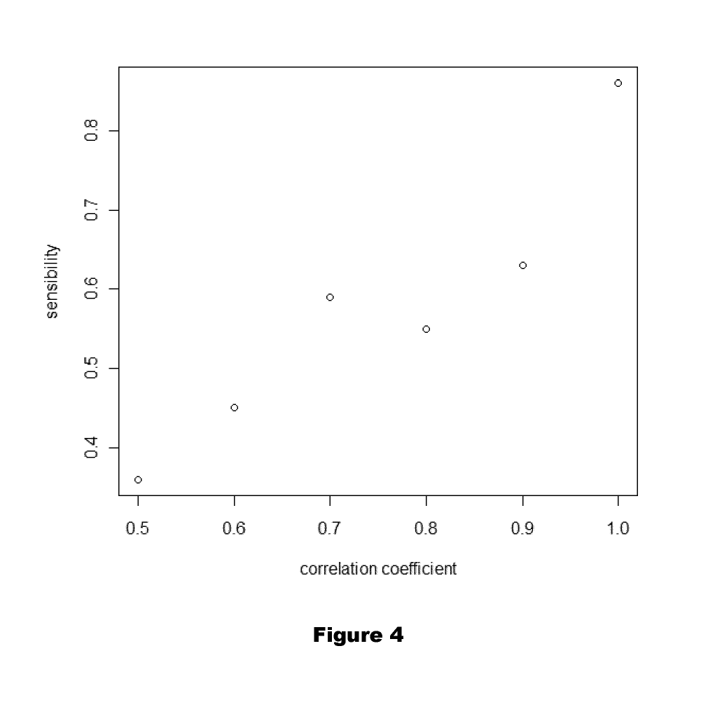

Correlation analysis is then further applied to the qPCR data to illustrate the impact of replacing genes by correlated genes on the discriminatory performance of the signature Tables 9, 10 and FIG. 4).

Table 9 below further lists genes whose expression levels, as measured by RT-qPCR in whole blood, correlate at more than 0.6 (Pearson's correlation coefficient superior to 0.6, p-val<0.0001) to 10 candidate genes. Those results were generated from the 90 samples described in Example 4.

TABLE 9
List of genes that correlate at more than 0.6 to the 10 candidate genes in the
header (in bold). The correlation coefficient is given in brackets. When a gene that
correlates at more than 0.6 (p-val < 0.0001) to one of the 10 candidate genes is itself a
candidate gene, it is indicated by the sign *.

| MKNK1 | PRF1 | TBX21 | IFNGR2 | FYN | IL1B | CFLAR | MAPK14 | GNLY | TGFBR3 |
|---|---|---|---|---|---|---|---|---|---|
| MAPK14 | TBX21 | CCR5 | IL8RB | ELK4 | IL1RN | IL8RB | IL8RB | TBX21 | ABCB1 |
| (0.65)* | (0.86)* | (0.7) | (0.78) | (0.74) | (0.64) | (0.75) | (0.78) | (0.75)* | (0.63) |
| CD11c | CCR5 | TGFBR3 | IL6R | ABCB1 | TLR5 | MAPK14 | MMP9 | TGFBR3 | TBX21 |
| (0.62) | (0.61) | (0.89)* | (0.65) | (0.61) | (0.63) | (0.67)* | (0.67) | (0.75)* | (0.89)* |
| IFNGR2 | TGFBR3 | CCL4 | IL1RN | CCR5 | | HCK0021 | IL6R | CCL4 | CCR5 |
| (0.61)* | (0.73)* | (0.72) | (0.62) | (0.62) | | 10(0.61) | (0.66) | (0.61) | (0.74) |
| IFITM2 | CCL4 | IL2RB | MAPK14 | TGFBR3 | | TGFBR2 | IL1RN | IL2RB | CCL4 |
| (0.61) | (0.66) | (0.69) | (0.78)* | (0.71)* | | (0.72) | (0.62) | (0.61) | (0.7) |
| | IL2RB | GNLY- | HCK0021 | IL2RB | | TNFAIP9 | HCK0021 | PRF1 | IL2RB |
| | (0.75) | probe 1 | 10(0.65) | (0.64) | | (0.71) | 10(0.61) | (0.68)* | (0.68) |
| | | (0.73)* | | | | | | | |
| | GNLY- | GNLY- | TGFBR2 | KLRK1 | | IFNGR2 | CD16 | | GNLY- |
| | probe 1 | probe 2 | (0.62) | (0.66) | | (0.67)* | (0.69) | | probe 1 |
| | (0.64)* | (0.75)* | | | | | | | (0.73)* |
| | GNLY- | PRF1 | CD16 | | | IL1R1 | CD11c | | ENPP4 |
| | probe 2 | (0.86)* | (0.67) | | | (0.65) | (0.72) | | (0.63) |
| | (0.68)* | | | | | | | | |
| | | | CD11c | | | IFITM2 | SLC2A3 | | KLRK1 |
| | | | (0.63) | | | (0.72) | (0.67) | | (0.71) |
| | | | TIMP2 | | | DECR1 | TIMP2 | | FYN |
| | | | (0.76) | | | (0.69) | (0.82) | | (0.71)* |
| | | | MKNK1 | | | | MKNK1 | | GNLY- |
| | | | (0.61)* | | | | (0.65)* | | probe 2 |
| | | | | | | | | | (0.75)* |
| | | | TNFAIP9 | | | | HSPA1A | | PRF1 |
| | | | (0.71) | | | | (0.62) | | (0.73)* |
| | | | CFLAR | | | | TNFAIP9 | | |
| | | | (0.67)* | | | | (0.74) | | |
| | | | CASC3 | | | | CFLAR | | |
| | | | (0.64) | | | | (0.67)* | | |
| | | | IFITM2 | | | | IFNGR2 | | |
| | | | (0.84) | | | | (0.78)* | | |
| | | | | | | | TLR5 | | |
| | | | | | | | 0.67) | | |
| | | | | | | | CASC3 | | |
| | | | | | | | (0.63) | | |
| | | | | | | | IFITM2 | | |
| | | | | | | | (0.8) | | |

To illustrate the equivalence of the performance between a signature based on 8 genes and a signature based on 8 genes with one or the other gene replaced by an equivalent gene (such as one from the list in Table 9), we have replaced PRF1 by IL2RB in the signature, or CFLAR by IFITM2. The resulting performances are only slightly lower as shown in Table 10 below, thus showing that replacing an original gene in a claimed gene combination by an equivalent gene as disclosed in the application still permits a reliable prognosis.

TABLE 10
PPV, NPV, sensitivity, specificity and
error rate for 3 gene signatures.

| | PPV | NPV | Sensitivity | Specificity | ER |
|---|---|---|---|---|---|
| 8 original genes | 0.486 | 0.939 | 0.857 | 0.708 | 0.244 |
| (MKNK1, PRF1, TBX21, | | | | | |
| TGFBR3, IFNGR2, FYN, | | | | | |
| IL1B and CFLAR) | | | | | |
| 8 genes with PRF1 replaced | 0.545 | 0.846 | 0.545 | 0.846 | 0.230 |
| by IL2RB | | | | | |
| (MKNK1, IL2RB, TBX21, | | | | | |
| TGFBR3, IFNGR2, FYN, | | | | | |
| IL1B and CFLAR) | | | | | |
| 8 genes with CFLAR | 0.469 | 0.875 | 0.682 | 0.742 | 0.273 |
| replaced by IFITM2 | | | | | |
| (MKNK1, PRF1, TBX21, | | | | | |
| TGFBR3, IFNGR2, FYN, | | | | | |
| IL1B and IFITM2) | | | | | |

The possibility to replace genes by equivalent ones based on their correlation coefficient is further illustrated in FIG. 4, which shows that replacing genes genes by equivalent ones with a correlation coefficient of at least 0.9 still permits prediction (prognosis) with reasonable sensitivity.

### Example 4

### Further Validation of Candidate Signatures and Further Signatures in an Increased Number of Patients

In Example 2, the signatures of responsiveness to Infliximab treatment in RA-patients have been tested in samples of 40 RA patients.

Similarly, a signature of responsiveness Infliximab treatment in RA-patients consisting of MAPK14 and GNLY disclosed in PCT application N° PCT/EP2011/054569, consisting of MAPK14 and GNLY, had been tested in this PCT application in the same samples of 40 RA patients.

To further validate these signatures, they have been further tested in a group of 90 RA patients treated with Infliximab corresponding to the previously tested RA patients and 50 additional RA patients. In addition, two further signatures corresponding to the 8 genes signature (MKNK1, PRF1, TBX21, TGFBR3, IFNGR2, FYN, IL1B and CFLAR) described above in Example 2, to which are added two further genes ((CD14 and TGFBR2) or (MAPK14 and GNLY)), have been further tested in the group of 90 patients.

Patients and Methods

Patients

Among the 90 tested patients, 40 are the same as those already tested in Example 2, additional 3 samples were included from the same source. Thus 42 samples used for the qCPR represent a subset of the original 43 samples used in the statistical analysis of microarray analysis in the study of Julia et al. One outlier in microarray data (sample 44) that was excluded from microarray analysis was not an outlier in the qPCR analysis and hence contributes as an independent sample. Therefore 43 (36 responders, 7 non responders) RNA samples from Julia et al. were used in the qPCR analysis. Additional 57 samples from RA patients meeting our inclusion criteria were obtained from three additional sites: 24 (14 responders, 10 non responders) from Japan, 14 (10 responders, 4 non responders) from the UK and 9 (8 responders, 1 non responders) from France. The delatadeltaCT method was used to measure gene expression levels using the same reference genes as in example 2.

Patient characteristics for each source are described in Table 11.

TABLE 11
Patient characteristics by response status and by contributing site.

| Clinical | Clinical | | | | | |
|---|---|---|---|---|---|---|
| Variable | Response | UK | Spain | France | Japan | Overall |
| DAS28 | Non | 6.60 | 5.76 | 3.71 | 5.11 | 5.52 |
| at | Responding | | | | | |
| baseline | Responding | 6.33 | 6.00 | 5.21 | 5.35 | 5.82 |
| DAS28 | Non | 6.10 | 5.44 | 3.41 | 5.25 | 5.38 |
| at week | Responding | | | | | |
| 14 | Responding | 3.07 | 3.79 | 3.83 | 2.87 | 3.5 |
| Delta | Non | 0.50 | 0.31 | 0.30 | −0.15 | 0.14 |
| DAS28 | Responding | | | | | |
| | Responding | 3.26 | 2.21 | 1.38 | 2.48 | 2.32 |
| Age at | Non | 64.25 | 57.71 | 44.00 | 59.10 | 58.91 |
| Initiation | Responding | | | | | |
| | Responding | 52.90 | 52.29 | 55.75 | 56.00 | 53.57 |
| Disease | Non | NA | NA | 9.00 | 8.00 | 8.09 |
| duration | Responding | | | | | |
| | Responding | NA | NA | 6.71 | 10.51 | 9.24 |
| Weight | Non | 64.00 | NA | NA | NA | 64.00 |
| | Responding | | | | | |
| | Responding | 75.90 | NA | NA | NA | 75.90 |
| ESR | Non | 37.75 | 38.29 | 15.00 | 54.30 | 44.41 |
| Baseline | Responding | | | | | |
| | Responding | 34.50 | 40.67 | 35.67 | 53.14 | 41.93 |

Methods

Table 12 below provides the Gene Symbol, the Gene Title, the Gene ID (Notation from Entrez Gene NCBI database, updated on 8 May 2011), and the reference mRNA sequence(s) (Entrez Gene NCBI database, updated on 8 May 2011) for all human genes present in one or more of the tested signatures:

TABLE 12
Gene Symbol, Gene Title and Gene ID (Notation from Entrez Gene NCBI database,
updated on December 2010) for genes CD14, TGFBR2, MAPK14 and GNLY

| Gene | | | |
|---|---|---|---|
| Symbol | Gene Title | Gene ID | Reference mRNA sequence(s) |
| MKNK1 | MAP kinase interacting | 8569 | NM_001135553.1 (SEQ ID NO: 1) |
| | serine/threonine | | NM_003684.4 (SEQ ID NO: 2) |
| | kinase 1 | | NM_198973.2 (SEQ ID NO: 3) |
| PRF1 | perforin 1 (pore | 5551 | NM_001083116.1 (SEQ ID NO4) |
| | forming protein) | | NM_005041.4 (SEQ ID NO: 5) |
| TBX21 | T-box 21 | 30009 | NM_013351.1 (SEQ ID NO: 6) |
| TGFBR3 | transforming growth | 7049 | NM_001195683.1 (SEQ ID NO: 7) |
| | factor, beta receptor III | | NM_001195684.1 (SEQ ID NO: 8) |
| | | | NM_003243.4 (SEQ ID NO: 9) |
| IFNGR2 | interferon gamma | 3460 | NM_005534.3 (SEQ ID NO: 10) |
| | receptor 2 (interferon | | |
| | gamma transducer 1) | | |
| FYN | FYN oncogene related | 2534 | NM_002037.3 (SEQ ID NO: 11) |
| | to SRC, FGR, YES | | NM_153047.1 (SEQ ID NO: 12) |
| | | | NM_153048.1 (SEQ ID NO: 13) |
| IL1B | interleukin 1, beta | 3553 | NM_000576.2 (SEQ ID NO: 14) |
| CFLAR | CASP8 and FADD-like | 8837 | NM_001127183.2 (SEQ ID NO: 15) |
| | apoptosis regulator | | NM_001127184.2 (SEQ ID NO: 16) |
| | | | NM_001202515.1 (SEQ ID NO: 17) |
| | | | NM_001202516.1 (SEQ ID NO: 18) |
| | | | NM_001202517.1 (SEQ ID NO: 19) |
| | | | NM_001202518.1 (SEQ ID NO: 20) |
| | | | NM_001202519.1 (SEQ ID NO: 21) |
| | | | NM_003879.5 (SEQ ID NO: 22) |
| CD14 | CD14 molecule | 929 | NM_000591.3 (SEQ ID NO: 23) |
| | | | NM_001040021.2(SEQ ID NO: 24) |
| | | | NM_001174104.1(SEQ ID NO: 25) |
| | | | NM_001174105.1(SEQ ID NO26:) |
| TGFBR2 | transforming growth | 7048 | NM_001024847.2 (SEQ ID NO: 27) |
| | factor, beta receptor II | | NM_003242.5 (SEQ ID NO: 28) |
| | (70/80 kDa) | | |
| MAPK14 | mitogen-activated | 1432 | NM_001315.2 (SEQ ID NO: 29) |
| | protein kinase 14 | | NM_139012.2 (SEQ ID NO: 30) |
| | | | NM_139013.2 (SEQ ID NO: 31) |
| | | | NM_139014.2 (SEQ ID NO: 32) |
| GNLY | granulysin | 10578 | NM_006433.3 (SEQ ID NO: 33) |
| | | | NM_012483.2(SEQ ID NO: 34) |

qPCR experiments have been performed as described in Example 2.

Cross-Validation Methods

To robustly estimate the performance of the 8 gene signature, we applied the signature of the 8 genes developed in example 2 to the 90 samples. In a first analysis 43 samples were used for the learning phase and the remaining 47 as an independent set. In a second approach we aimed at improving on the existing signature by adding additional genes to improve the discriminatory performance. For this second approach we used leave one out cross validation on the entire 90 samples.

Results and Discussion

The prognosis of a non-responding condition (to Infliximab) was analyzed. As a result, a non-responding condition or test outcome is considered a positive result, while a responding condition or test outcome is considered a negative result. True and false positive results, NPV, PPV, specificity sensitivity, and error rate are defined and calculated as follows:

| | | Condition (non-responding) | |
|---|---|---|---|
| | | Positive | Negative |
| Test outcome | Positive | True Positive (TP) | False positive (FP) |
| (non-responding) | Negative | False negative (FN) | True negative (TN) |

PPV=TP/(TP+FP)

NPV=TN/(TN+FN)

Specificity=TN/(TN+FP)

Sensitivity=TP/(TP+FN)

Error rate=(FP+FN)/Total number of patients

The main clinical objective of a signature is not to put patients under inefficacious treatment, therefore a signature should demonstrate a low level of False Negative results.

Results obtained on at least a subgroup of the 90 RA patients are provided below for:
the 8 genes signature (MKNK1, PRF1, TBX21, TGFBR3, IFNGR2, FYN, IL1B and CFLAR) already tested in Example 2 on 40 RA patients (see Table 13),
the 2 genes signature (MAPK14 and GNLY) previously tested in PCT application PCT/EP2011/054569 on 40 RA patients (see Table 14),
the 10 genes signature (MKNK1, PRF1, TBX21, TGFBR3, IFNGR2, FYN, IL1B, CFLAR, CD14 and TGFBR2), comprising the 8 genes tested in Example 2 and two additional genes (CD14 and TGFBR2) (see Table 15),
the 10 genes signature (MKNK1, PRF1, TBX21, TGFBR3, IFNGR2, FYN, IL1B, CFLAR, MAPK14 and GNLY), combining the 8 genes already tested in Example 2 on 40 RA patients and the two genes already tested in PCT application PCT/EP2011/054569 on the same 40 RA patients (see Table 16),
The 1 gene signature MKNK1 (see Table 17),
The 2 genes signature (MKNK1, GNLY) (see Table 17),
The 3 genes signature (MKNK1, GNLY, ADI1) (see Table 17),
The 4 genes signature (MKNK1, GNLY, ADI1, IL1B) (see Table 17), and
The 5 genes signature (MKNK1, GNLY, ADI1, IL1B, IL1R1) (see Table 17).

Samples for which there was missing data based on invalid qPCR results were excluded.

TABLE 13
Results obtained with the 8 genes signature (MKNK1, PRF1,
TBX21, TGFBR3, IFNGR2, FYN, IL1B and CFLAR) in 86 patients

| | | Condition (non-responding) | | |
|---|---|---|---|---|
| | | Positive | Negative | |
| Test outcome | Positive | 18 | 19 | 0.486 PPV |
| (non-responding) | Negative | 3 | 46 | 0.939 NPV |
| | | 0.857 Sens | 0.708 Spe | |

Error rate = 0.256
Positive = non-responding to Infliximab treatment
Negative = responding to Infliximab treatment
PPV = Positive Predictive value
NPV = Negative Predictive value
Sens = sensitivity
Spe = specicifity

TABLE 14
Results obtained with the 2 genes signature
(MAPK14, GNLY) in 90 patients

| | | Condition (non-responding) | | |
|---|---|---|---|---|
| | | Positive | Negative | |
| Test outcome | Positive | 10 | 3 | 0.769 PPV |
| (non-responding) | Negative | 12 | 65 | 0.844 NPV |
| | | 0.455 Sens | 0.956 Spe | |

Error rate = 0.167
Positive = non-responding to Infliximab treatment
Negative = responding to Infliximab treatment
PPV = Positive Predictive value
NPV = Negative Predictive value
Sens = sensitivity
Spe = specicifity

TABLE 15
Results obtained with the first 10 genes signature
(MKNK1, PRF1, TBX21, TGFBR3, IFNGR2, FYN, IL1B,
CFLAR, CD14 and TGFBR2) in 75 patients

| | | Condition (non-responding) | | |
|---|---|---|---|---|
| | | Positive | Negative | |
| Test outcome | Positive | 10 | 5 | 0.667 PPV |
| (non-responding) | Negative | 8 | 52 | 0.867 NPV |
| | | 0.556 Sens | 0.912 Spe | |

Error rate = 0.173
Positive = non-responding to Infliximab treatment
Negative = responding to Infliximab treatment
PPV = Positive Predictive value
NPV = Negative Predictive value
Sens = sensitivity
Spe = specicifity

TABLE 16
Results obtained with the second 10 genes signature (MKNK1,
PRF1, TBX21, TGFBR3, IFNGR2, FYN, IL1B, CFLAR, MAPK14 and
GNLY) in 60 patients. In this particular case a three level
score was used with samples classifying as intermediate
risk of response, not being represented here.

| | | Condition (non-responding) | | |
|---|---|---|---|---|
| | | Positive | Negative | |
| Test outcome | Positive | 9 | 2 | 0.818 PPV |
| (non-responding) | Negative | 3 | 46 | 0.939 NPV |
| | | 0.750 Sens | 0.958 Spe | |

Error rate = 0.083
Positive = non-responding to Infliximab treatment
Negative = responding to Infliximab treatment
PPV = Positive Predictive value
npv = Negative Predictive value
Sens = sensitivity
Spe = specicifity

The values of PPV, NPV, sensitivity and specificity of all four tested signatures are summarized in Table 17 below:

TABLE 17
Summary of PPV, NPV, specificity, sensitivity and error rate of prognosis
methods of a non-responding condition to Infliximab

| | Signature | PPV | NPV | Sensitivity | Specificity | Error rate |
|---|---|---|---|---|---|---|
| 1 | 8 genes (MKNK1, PRF1, | 0.486 | 0.939 | 0.857 | 0.708 | 0.256 |
| | TBX21, TGFBR3, IFNGR2, | | | | | |
| | FYN, IL1B and CFLAR) | | | | | |
| 2 | 2 genes (MAPK14, GNLY) | 0.769 | 0.844 | 0.455 | 0.956 | 0.167 |
| 3 | 10 genes (MKNK1, PRF1, | 0.667 | 0.867 | 0.556 | 0.912 | 0.173 |
| | TBX21, TGFBR3, IFNGR2, | | | | | |
| | FYN, IL1B, CFLAR, CD14 and | | | | | |
| | TGFBR2) | | | | | |
| 4 | 10 genes (MKNK1, PRF1, | 0.818 | 0.939 | 0.75 | 0.958 | 0.083 |
| | TBX21, TGFBR3, IFNGR2, | | | | | |
| | FYN, IL1B, CFLAR, MAPK14 | | | | | |
| | and GNLY) | | | | | |
| 5 | 1 gene (MKNK1) | 0.57 | 0.78 | 0.18 | 0.96 | 0.23 |
| 6 | 2 genes (MKNK1, GNLY) | 0.65 | 0.85 | 0.5 | 0.91 | 0.19 |
| 8 | 3 genes (MKNK1, GNLY, | 0.79 | 0.86 | 0.5 | 0.96 | 0.16 |
| | ADI1) | | | | | |
| 7 | 4 genes (MKNK1, GNLY, | 0.81 | 0.88 | 0.59 | 0.96 | 0.13 |
| | ADI1, IL1B) | | | | | |
| 9 | 5 genes (MKNK1, GNLY, | 0.82 | 0.89 | 0.63 | 0.96 | 0.12 |
| | ADI1, IL1B, IL1R1) | | | | | |

Data summarized in Table 17 show that the first four tested signatures have high NPV value for prognosis of non-responding condition, meaning that, in each case, a high proportion of patients with a negative test (i.e. prognosed as responding to Infliximab) actually do respond to the Infliximab treatment, so that, based on these prognosis methods of non-responding condition, there will be very few patients prognosed as responding that will fail to respond to the treatment. Thus, based on these prognosis methods, Infliximab will be administered almost only to patients that will actually respond to the treatment. The first four tested signatures also have an acceptable or high specificity for prognosis of non-responding condition, meaning that a high proportion of patients that would actually respond to Infliximab treatment are correctly prognosed as responders using the signatures. Thus, based on the above prognosis methods of non-responding condition according to the invention, most patients that would benefit from Infliximab treatment will actually receive this treatment. The two signatures of greatest clinical interest are the initial eight gene signature as well as the combined 10 genes signature that include MAPK14 and GNLY where the sensitivity is higher than in the other two.

The first four tested signatures are thus helpful prognosis tools for identifying RA patients that will or not respond to an Infliximab treatment.

In addition, data obtained for the 8 genes signature (MKNK1, PRF1, TBX21, TGFBR3, IFNGR2, FYN, IL1B and CFLAR) and the two 10 genes signatures (MKNK1, PRF1, TBX21, TGFBR3, IFNGR2, FYN, IL1B, CFLAR, CD14 and TGFBR2/MKNK1, PRF1, TBX21, TGFBR3, IFNGR2, FYN, IL1B, CFLAR, MAPK14 and GNLY), which both include the 8 genes signature, confirm that the addition of one or more genes to this 8 genes signature does not affect its ability to correctly prognose RA patients as responders or non responders to Infliximab treatment. In contrast, the 10 genes signature combining the 8 genes signature of Example 2 and the 2 genes signature of PCT application PCT/EP2011/054569 shows particularly high NPV and specificity values (the highest of all four signatures), while keeping quite high PPV (the highest of all four signatures) and sensitivity (second best of all four signatures) values. This 10 genes signature (MKNK1, PRF1, TBX21, TGFBR3, IFNGR2, FYN, IL1B, CFLAR, MAPK14 and GNLY) appears to be a particularly good prognosis tool of response and non response to Infliximab treatment in RA patients. All the above methods may also be used for prognosis of non response to other TBA treatments, as well as other CyTD treatments.

In addition, Table 17 also shows that MKNK1 alone permits classification of patients with an error rate of only 0.23. In particular, a prognosis based on MKNK1 is highly specific and also has a high NPV value. Moreover, the addition of at least one other gene significantly improves all values (PPV, NPV, sensitivity, specificity, error rate), and a NPV value of at least 0.85 is obtained when at least one gene is added to MKNK1. These results confirm that MKNK1 is a key gene for the prediction of response or non-response to Infliximab and that gene signatures based on MKNK1 and preferably at least one more gene may result in reliable prediction.

In summary, several signatures (or genes combinations) useful for prognosis of non-responsiveness to CyTD treatment (in particular Infliximab treatment) in subjects suffering from an inflammatory disease (in particular rheumatoid arthritis, RA) have been identified, three of which comprising the 8 following genes: MKNK1, PRF1, TBX21, TGFBR3, IFNGR2, FYN, IL1B and CFLAR. The best signature identified so far for prognosis of non-responsiveness to CyTD treatment (in particular Infliximab treatment) in subjects suffering from an inflammatory disease (in particular RA) comprises or consists of genes MKNK1, PRF1, TBX21, TGFBR3, IFNGR2, FYN, IL1B, CFLAR, MAPK14 and GNLY.

### Example 5

### Further Validation of a Highly Discriminant 11 Gene Signature Using a Linear Model

Example of a highly discriminant algorithm is provided, it can be applied at baseline (ie prior to treatment administration) to predict whether a patient will be a responder or a non responder to Infliximab. In this example the biological measures are based on gene expression profiles of 11 genes as measured by relative quantitative PCR on RNA extracted from peripheral blood. Those measures are then combined in a linear model to provide a score, a positive score means that the patient is predicted to be a responder. Such algorithm can easily be implemented in existing software or coded specifically.

Patients and Methods

Patients

In this example 65 Caucasian RA patients were analysed. All 65 patients were eligible for first line anti-TNF therapy, as such they were naive of biologics and refractory to classical DMARDS. The 65 patients have already been described in example 4 (the samples from France, Spain and UK).

Methods

The delatadeltaCT method was used to measure gene expression levels using the same reference genes and procedures as in examples 2 and 4. Previous Table 12 provides the Gene Symbol, the Gene Title, the Gene ID (Notation from Entrez Gene NCBI database, updated on 8 May 2011), and the reference mRNA sequence(s) (Entrez Gene NCBI database, updated on 8 May 2011) of 12 genes, 11 of which the Applied probes used in this example map to. Table 18 below provides the Applied Biosystems probe identifiers.

TABLE 18
Probe identifiers for an 11 gene signature

| Gene | | |
|---|---|---|
| Symbol | Probeid | Probe sequence |
| CD14 | h_CD14_Hs02621496_ | GGAATGGAGACGCCCACAG |
| | s1_FAM | GCGTGT |
| | | (SEQ ID NO: 35) |
| CFLAR | h_CFLAR_Hs01116280_ | TAAGCAAGGAGAAGAGTTT |
| | m1_FAM | CTTGGA |
| | | (SEQ ID NO: 35) |
| IFNGR2 | h_IFNGR2_Hs00194264_ | CGGAATGTGACTGTCGGGC |
| | m1_FAM | CTCCAG |
| | | (SEQ ID NO: 37) |
| IL1B | h_IL1B_Hs01555410_ | GATGAAGTGCTCCTTCCAG |
| | m1_FAM | GACCTG |
| | | (SEQ ID NO: 38) |
| GNLY | h_GNLY_Hs01120727_ | TACCTTCTACAGGTCCCCT |
| | m1_FAM | CTGAGC |
| | | (SEQ ID NO: 39) |
| MAPK14 | h_MAPK14_315_ | TGTTTCCTGGTACAGACCA |
| | Hs01051153_m1_FAM | TATTAA |
| | | (SEQ ID NO: 40) |
| MKNK1 | h_MKNK1_Hs00374376_ | CCACCCCATGTGGCTCTGC |
| | m1_FAM | AGAATA |
| | | (SEQ ID NO: 41) |
| PRF1 | h_PRF1_Hs00169473_ | TGCCGCTTCTACAGTTTCC |
| | m1_FAM | ATGTGG |
| | | (SEQ ID NO: 42) |
| TBX21 | h_TBX21_Hs00894392_ | ACAATGTGACCCAGATGAT |
| | m1_FAM | TGTGCT |
| | | (SEQ ID NO: 43) |
| TGFBR2 | h_TGFBR2_Hs00234253_ | TGGCTCAACCACCAGGGCA |
| | m1_FAM | TCCAGA |
| | | (SEQ ID NO: 44) |
| TGFBR3 | h_TGFBR3_Hs01114253_ | CACTGCAGGTCCAGAGCCT |
| | m1_FAM | GGTGCA |
| | | (SEQ ID NO: 45) |

Results and Discussion

Score=−2608.15−588.10*ddCT_{MKMK1}+218.42*ddCT_{TBX21}+211.81*ddCT_{TGFBR3}−99.48*ddCT_{PRF1}+325.63*ddCT_{IFNGR2}−247.78*ddCT_{IL1B}+272.59*ddCT_{CFLAR}−896.87*ddCT_{TGFBR2}−439.47*ddCT_{CD14}+69.04*ddCT_{GNLY}+480.50*ddCT_{MAPK14}

Equation 1: This equation provides a mean to estimate the probability that a patient is a responder or non responder to infliximab.

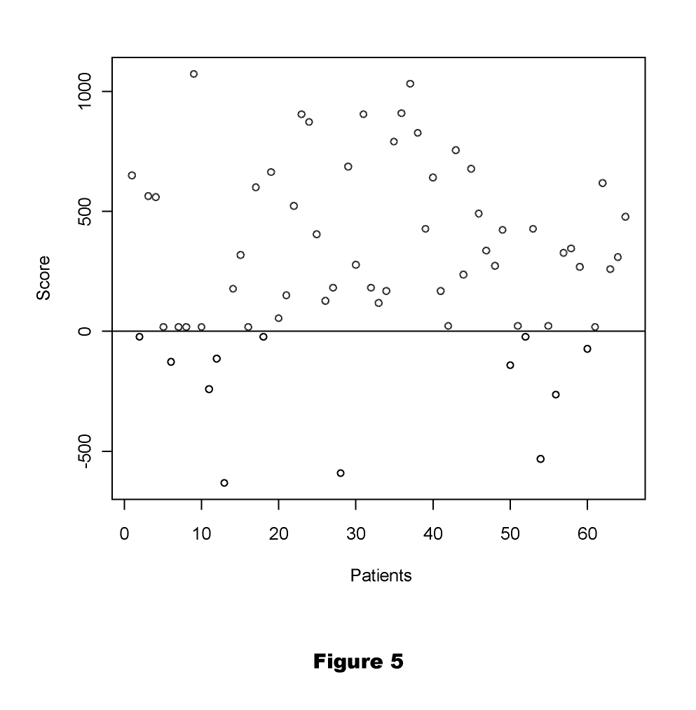

The coefficients in Equation 1 were estimated based on a training of the 65 samples. The score resulting from the linear model described in equation 1 allows to perfectly discriminate all responders from non responders as can be seen in FIG. 5. All patients above the horizontal line at zero are clinical responders to infliximab and predicted as such by a positive score, all non responders are below the line and predicted as such (negative score).

### REFERENCES

1. Lee D M, Weinblatt M E. Rheumatoid arthritis. Lancet. 2001 Sep. 15; 358(9285):903-11.

2. Choy E H, Panayi G S. Cytokine pathways and joint inflammation in rheumatoid arthritis. N Engl J Med. 2001 Mar. 22; 344(12):907-16.

3. Kooloos W M, de Jong D J, Huizinga T W, Guchelaar H J. Potential role of pharmacogenetics in anti-TNF treatment of rheumatoid arthritis and Crohn's disease. Drug Discovery Today. 2007; 12(3-4):125-31.

4. Isaacs J D. Antibody engineering to develop new antirheumatic therapies. Arthritis Res Ther. 2009; 11(3):225.

5. Hetland M L, Christensen I J, Tarp U, Dreyer L, Hansen A, Hansen I T, et al. Direct comparison of treatment responses, remission rates, and drug adherence in patients with rheumatoid arthritis treated with adalimumab, etanercept, or infliximab: results from eight years of surveillance of clinical practice in the nationwide Danish DANBIO registry. Arthritis Rheum. 2010 January; 62(1):22-32.

6. Lequerre T, Gauthier-Jauneau A C, Bansard C, Derambure C, Hiron M, Vittecoq O, et al. Gene profiling in white blood cells predicts infliximab responsiveness in rheumatoid arthritis. Arthritis Res Ther. 2006; 8(4):R105.

7. Sekiguchi N, Kawauchi S, Furuya T, Inaba N, Matsuda K, Ando S, et al. Messenger ribonucleic acid expression profile in peripheral blood cells from RA patients following treatment with an anti-TNF-alpha monoclonal antibody, infliximab. Rheumatology (Oxford). 2008 June; 47(6):780-8.

8. Julia A, Erra A, Palacio C, Tomas C, Sans X, Barcelo P, et al. An eight-gene blood expression profile predicts the response to infliximab in rheumatoid arthritis. PLoS One. 2009; 4(10):e7556.

9. Bienkowska J R, Dalgin G S, Batliwalla F, Allaire N, Roubenoff R, Gregersen P K, et al. Convergent Random Forest predictor: methodology for predicting drug response from genome-scale data applied to anti-TNF response. Genomics. 2009 December; 94(6):423-32.

10. Ramasamy A, Mondry A, Holmes C C, Altman D G. Key issues in conducting a meta-analysis of gene expression microarray datasets. PLoS Med. 2008 Sep. 30; 5(9):e184.

11. Arnett F C, Edworthy S M, Bloch D A, McShane D J, Fries J F, Cooper N S, et al. The American Rheumatism Association 1987 revised criteria for the classification of rheumatoid arthritis. Arthritis Rheum. 1988 March; 31(3):315-24.

12. Parmigiani G, Garrett-Mayer E S, Anbazhagan R, Gabrielson E. A cross-study comparison of gene expression studies for the molecular classification of lung cancer. Clin Cancer Res. 2004 May 1; 10(9):2922-7.

13. Barton A, Thomson W, Ke X, Eyre S, Hinks A, Bowes J, et al. Rheumatoid arthritis susceptibility loci at chromosomes 10p15, 12q13 and 22q13. Nat Genet. 2008 October; 40(10):1156-9.

14. Goronzy J J, Weyand C M. Developments in the scientific understanding of rheumatoid arthritis. Arthritis Res Ther. 2009; 11(5):249.

15. Lorenz E, Muhlebach M S, Tessier P A, Alexis N E, Duncan Hite R, Seeds M C, et al. Different expression ratio of S100A8/A9 and S100A12 in acute and chronic lung diseases. Respir Med. 2008 April; 102(4):567-73.

16. Cheng P, Corzo C A, Luetteke N, Yu B, Nagaraj S, Bui M M, et al. Inhibition of dendritic cell differentiation and accumulation of myeloid-derived suppressor cells in cancer is regulated by S100A9 protein. J Exp Med. 2008 Sep. 29; 205(10):2235-49.

17. Lim S Y, Raftery M, Goyette J, Hsu K, Geczy C L. Oxidative modifications of S100 proteins: functional regulation by redox. J Leukoc Biol. 2009 86(3): 577-87.

18. Simard J C, Girard D, Tessier P A. Induction of neutrophil degranulation by S100A9 via a MAPK-dependent mechanism. J Leukoc Biol. 2010 Jan. 26.

19. Chen Y S, Yan W, Geczy C L, Brown M A, Thomas R. Serum levels of soluble receptor for advanced glycation end products and of S100 proteins are associated with inflammatory, autoantibody, and classical risk markers of joint and vascular damage in rheumatoid arthritis. Arthritis Res Ther. 2009; 11(2):R39.

20. Groh V, Bruhl A, El-Gabalawy H, Nelson J L, Spies T. Stimulation of T cell autoreactivity by anomalous expression of NKG2D and its MIC ligands in rheumatoid arthritis. Proc Natl Acad Sci USA. 2003 Aug. 5; 100(16):9452-7.

21. Paul R, Obermaier B, Van Ziffle J, Angele B, Pfister H W, Lowell C A, et al. Myeloid Src kinases regulate phagocytosis and oxidative burst in pneumococcal meningitis by activating NADPH oxidase. J Leukoc Biol. 2008 October; 84(4):1141-50.

22. Fumagalli L, Zhang H, Baruzzi A, Lowell C A, Berton G. The Src family kinases Hck and Fgr regulate neutrophil responses to N-formyl-methionyl-leucyl-phenylalanine. J Immunol. 2007 Mar. 15; 178(6):3874-85.

23. Mocsai A, Ligeti E, Lowell C A, Berton G. Adhesion-dependent degranulation of neutrophils requires the Src family kinases Fgr and Hck. J Immunol. 1999 Jan. 15; 162(2):1120-6.

24. Bosco M C, Curiel R E, Zea A H, Malabarba M G, Ortaldo J R, Espinoza-Delgado I. IL-2 signaling in human monocytes involves the phosphorylation and activation of p59hck. J Immunol. 2000 May 1; 164(9):4575-85.

25. Deng A, Chen S, Li Q, Lyu S C, Clayberger C, Krensky A M. Granulysin, a cytolytic molecule, is also a chemoattractant and proinflammatory activator. J Immunol. 2005 May 1; 174(9):5243-8.

26. Krensky A M, Clayberger C. Biology and clinical relevance of granulysin. Tissue Antigens. 2009 March; 73(3):193-8.

27. Martinon F, Tschopp J. Inflammatory caspases and inflammasomes: master switches of inflammation. Cell Death Differ. 2007 January; 14(1):10-22.

28. Kurokawa M, Kornbluth S. Caspases and kinases in a death grip. Cell. 2009 Sep. 4; 138(5):838-54.

29. Morel J, Audo R, Hahne M, Combe B. Tumor necrosis factor-related apoptosis-inducing ligand (TRAIL) induces rheumatoid arthritis synovial fibroblast proliferation through mitogen-activated protein kinases and phosphatidylinositol 3-kinase/Akt. J Biol Chem. 2005 Apr. 22; 280(16):15709-18.

30. Korb A, Tohidast-Akrad M, Cetin E, Axmann R, Smolen J, Schett G. Differential tissue expression and activation of p38 MAPK alpha, beta, gamma, and delta isoforms in rheumatoid arthritis. Arthritis Rheum. 2006 September; 54(9):2745-

[symbol]

31. Fransen J, van Riel PLCM. The Disease Activity Score and the EULAR response criteria. Clin Exp Rheumatol. 2005 23(5 Suppl 39): S93-9.

32. Lorenz H M et al. Arthritis Res. 2002; 4 Suppl 3:S17-24

33. Atzeni F et al. Autoimmun Rev. 2007 September; 6(8):529-36.

34. Choi H et al. A latent variable approach for meta-analysis of gene expression data from multiple microarray experiments. BMC Bioinformatics. 2007 September; 8(364).

35. Choi H et al. Latent variable modelling for combining genomic data from multiple studies. Unpublished manuscript (2005).

36. Tanino M et al. Prediction of efficacy of anti-TNF biologic agent, infliximab, for rheumatoid arthritis patients using a comprehensive transcriptome analysis of white blood cells. Biochem Biophys Res Commun. 2009 September; 387(2):261-5.

37. van Baarsen L G et al. Regulation of IFN response gene activity during infliximab treatment in rheumatoid arthritis is associaed with clinical response to treatment. Arthritis Res Ther. 2010; 12(1):R11;

38. Setubal and Meidanis et al., Introduction to Computational Biology Methods (PWS Publishing Company, Boston, 1997;

39. Salzberg, Searles, Kasif, (Ed.), Computational Methods in Molecular Biology, (Elsevier, Amsterdam, 1998;

40. Rashidi and Buehler, Bioinformatics Basics: Application in Biological Science and Medicine (CRC Press, London, 2000;

41. Ouelette and Bzevanis Bioinformatics: A Practical Guide for Analysis of Gene and Proteins (Wiley & Sons, Inc., 2^{nd} ed., 2001.

1. A method for the in vitro diagnosis or prognosis of a cytokine targeting drug (CyTD) or anti-inflammatory biological drug responding or non-responding phenotype, comprising:
(a) determining from a biological sample of a subject suffering from an inflammatory disease an expression profile comprising or consisting of:
(i)
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s),
(b) comparing the obtained expression profile with at least one reference expression profile, and
(c) determining the responding or non-responding phenotype from said comparison.

2. A method for designing a CyTD or anti-inflammatory biological drug treatment for a subject suffering from an inflammatory disease, said method comprising:
(a) determining from a biological sample of said subject an expression profile comprising or consisting of:
(i)
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s),
(b) comparing the obtained expression profile with at least one reference expression profile,
(c) determining the responding or non-responding phenotype of said subject from said comparison, and
(d) designing a dose of CyTD or anti-inflammatory biological drug treatment according to the said identified responding or non-responding phenotype.

3. A method for adapting the CyTD or anti-inflammatory biological drug treatment of a subject suffering from an inflammatory disease, said method comprising:
(a) determining from a biological sample of said subject an expression profile comprising or consisting of:
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s), comparing the obtained expression profile with at least one reference expression profile,
(b) determining the responding or non-responding phenotype of said subject from said comparison, and
(c) adapting the CyTD or anti-inflammatory biological drug treatment.

4. A method of treatment of an RA-suffering subject, comprising the steps of:
(a) administering a therapeutic dose of a disease-modifying anti-rheumatic drug (DMARD) to the said subject suffering from RA,
(b) determining from a biological sample of a RA-suffering subject an expression profile comprising or consisting of:
(i)
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s),
(c) comparing the obtained expression profile with at least one reference expression profile,
(d) determining the responding or non-responding phenotype of said RA-suffering subject from said comparison,
(e) determining a dose of CyTD or anti-inflammatory biological drug, and
(f) administering the said dose of CyTD or anti-inflammatory biological drug.

5. The method of claim 4, wherein the DMARD is methotrexate.

6. A method of treatment of a subject suffering from an inflammatory disease, comprising the steps of:
(a) determining from a biological sample of said subject an expression profile comprising or consisting of:
(i)
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s),
(b) comparing the obtained expression profile with at least one reference expression profile,
(c) determining the responding or non-responding phenotype of said subject from said comparison,
(d) determining a dose of CyTD or anti-inflammatory biological drug, and
(e) administering the said dose of CyTD or anti-inflammatory biological drug.

7. The method of claim 2, wherein the expression profile of step (a) is determined at 14, 16 or 22 weeks after the beginning of the CyTD or antiinflammatory biological drug treatment.

8. The method of claim 1, wherein the obtained expression profile is compared to at least one reference responding and/or non-responding expression profile in step (b).

9. The method of claim 8, wherein the obtained expression profile is compared in step (b) to at least one reference responding expression profile and at least one reference non-responding expression profile.

10. The method of claim 9, wherein the comparison of the obtained expression profile to said at least one reference responding expression profile and at least one reference non-responding expression profile is performed using an algorithm selected from linear regression and derivatives thereof, including generalized linear model such as logistic regression; nearest neighbour (k-NN); decision trees; support vector machines (SVM); neural networks; linear discriminant analyses (LDA); random forests; or Predictive Analysis of Microarrays (PAM), wherein said algorithm has been calibrated based on said at least one reference responding expression profile and at least one reference non-responding expression profile.

11. The method of claim 1, wherein the expression profile is determined by measuring the amount of nucleic acid transcripts of said gene(s).

12. The method of claim 11, wherein the expression profile is determined using quantitative PCR or an oligonucleotide microarray.

13. The method of claim 1, wherein the expression profile is determined using a genomic microarray or a proteic microarray.

14. The method according to claim 1, wherein said biological sample is a blood sample.

15. The method according to claim 1, wherein said CyTD or anti-inflammatory biological drug is a TNF-a blocking agent (TBA), or a recombinant protein inhibitor of T cell costimulation by antigen presenting cells.

16. The method according to claim 1, wherein said inflammatory disease is selected from the group consisting of rheumatoid arthritis (RA), Crohn's disease, ankylosing spondylitis, psoriatic arthritis, plaque psoriasis, ulcerative colitis, vasculitis; Wegener's granulomatosis; sarcoidosis; adult-onset Still's disease, polymyositis/dermatomyositis, and systemic lupus erythematosus (SLE).

17. The method of claim 16, wherein said inflammatory disease is rheumatoid arthritis.

18. The method according to claim 17, further comprising determining at least one additional parameter, said additional parameter being determined by a test selected from the Antinuclear Antibody test (ANA test), C-Reactive Protein test (CRP test), Cyclic Citrullinated Peptide Antibody test (CCP test), and the Rheumatoid Factor test.

19. A kit for the in vitro diagnosis or prognosis of a CyTD or anti-inflammatory biological drug responding or non responding phenotype, comprising at least one reagent for the determination of an expression profile comprising or consisting of:
(i)
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s).

20. The kit of claim 19, further comprising at least one reagent for determining at least one additional parameter, said reagent selected from the Antinuclear Antibody test (ANA test), C-Reactive Protein test (CRP test), Cyclic Citrullinated Peptide Antibody test (CCP test), and the Rheumatoid Factor test.

21. A nucleic acid microarray comprising nucleic acids specific for:
(i)
the gene MKNK1; or
the genes MKNK1 and GNLY; or
the genes MKNK1, TBX21 and TGFBR3; or
the genes MKNK1, GNLY, and ADI1; or
the genes MKNK1, GNLY, ADI1, and IL1B; or
the genes MKNK1, GNLY, ADI1, IL1B, and IL1R1; or
the genes MKNK1, PRF1, TBX21, TGFBR3, IFNGR2, FYN, IL1B and CFLAR; or
the genes MKNK1, PRF1, TBX21, TGFBR3, IFNGR2, FYN, IL1B, CFLAR, MAPK14 and GNLY; or
the genes MKNK1, PRF1, TBX21, TGFBR3, IFNGR2, FYN, IL1B, CFLAR, CD14 and TGFBR2; or
the genes MKNK1, IFNGR2, IL1B, MAPK14, GNLY, and CD14; or
the genes MKNK1, PRF1, TBX21, TGFBR3, IFNGR2, IL1B, CFLAR, MAPK14, GNLY, CD14 and TGFBR2; or
all the 46 genes of Tables 2, 3 and 4; or
Equivalent Expression Profile thereof, provided that, in said Equivalent Expression Profile thereof, MKNK1 is not replaced by gene S 100A8 nor gene MAPK14, and
(ii) optionally one or more housekeeping gene(s).

22. The nucleic acid microarray according to claim 21, which is an oligonucleotide microarray.

23. A CyTD or anti-inflammatory biological drug, for use in treating an inflammatory disease, wherein the CyTD or anti-inflammatory biological drug is administered to a subject suffering from said inflammatory disease who has been diagnosed and/or prognosed as responsive using a method according to claim 1.

24. A CyTD according to claim 23, which is a TNF alpha binding agent.


SEQUENCE LISTING (45 sequences)

SEQ ID NO: 1 - DNA, 2693 bp, Homo sapiens
  misc_feature: MKNK1
tctctgatcgggcccaccccctggatctagcaccgcctcttccgcgttctcggaggagcgatctgcaggtaggggtgcgcgcgaccgctccccggcgggagccagcgaaggtttccatgtcagaggccgatggagaactgaagattgccacctacgcacaaaggccattgagacacttcgtgtagctggaagacaccaacttcctgacaggagctttatttcatttgggatttcaagtttacagatggtatcttctcaaaagttggaaaaacctatagagatgggcagtagcgaaccccttcccatcgcagatggtgacaggaggaggaagaagaagcggaggggccgggccactgactccttgccaggaaagtttgaagatatgtacaagctgacctctgaattgcttggagagggagcctatgccaaagttcaaggtgccgtgagcctacagaatggcaaagagtatgccgtcaaaatcatcgagaaacaagcagggcacagtcggagtagggtgtttcgagaggtggagacgctgtatcagtgtcagggaaacaagaacattttggagctgattgagttctttgaagatgacacaaggttttacttggtctttgagaaattgcaaggaggttccatcttagcccacatccagaagcaaaagcacttcaatgagcgagaagccagccgagtggtgcgggacgttgctgctgcccttgacttcctgcataccaaaggcattgctcatcgtgatctgaaaccagaaaatatattgtgtgaatctccagaaaaggtgtctccagtgaaaatctgtgactttgacttgggcagtgggatgaaactgaacaactcctgtacccccataaccacaccagagctgaccaccccatgtggctctgcagaatacatggcccctgaggtagtggaggtcttcacggaccaggccacattctacgacaagcgctgtgacctgtggagcctgggcgtggtcctctacatcatgctgagtggctacccacccttcgtgggtcactgcggggccgactgtggctgggaccggggcgaggtctgcagggtgtgccagaacaagctgtttgaaagcatccaggaaggcaagtatgagtttcctgacaaggactgggcacacatctccagtgaagccaaagacctcatctccaagctcctggtgcgagatgcaaagcagagacttagcgccgcccaagttctgcagcacccatgggtgcaggggcaagctccagaaaagggactccccacgccgcaagtcctccagaggaacagcagcacaatggacctgacgctcttcgcagctgaggccatcgcccttaaccgccagctatctcagcacgaagagaacgaactagcagaggagccagaggcactagctgatggcctctgctccatgaagctttcccctccctgcaagtcacgcctggcccggagacgggccctggcccaggcaggccgtggtgaagacaggagcccgcccacagcactctgaaatgctccagtcacaccttataggccctaggcctggccaggcattgtcccctggaaacctgtgtggctaaagtctgctgagcaggcagcagcctctgctctgtggctccattcaggctttttcatctacgaaggccctgaggttcccatcaacccccatttccctagggtcctggaggaaaaagctttttccaaaggggttgtctttgaaaaggaaagcaatcacttctcactttgcataattgcctgcagcaggaacatctcttcactgggctccacctgctcacccgcctgcagatctgggatccagcctgctctcaccgctgtagctgtggcggctggggctgcagcctgcagggagaagcaagaagcatcagttgacagaggctgccgacacgtgcctcttccctctcttctctgtcaccctcctctggcggtccttccaccttcctctgtcctccggatgtcctctttgcccgtcttctcccttggctgagcaaagccatcccctcaattcagggaagggcaaggagccttcctcattcaggaaatcaaatcagtcttccggtctgcagcacggaaaagcacataatctttctttgctgtgactgaaatgtatccctcgtttatcatcccctttgtttgtgattgctgctaaagtcagtagtatcgtttttttaaaaaaaaagtttggtgtttttaaccatgctgttccagcaaagatgataccttaaactcccactgcaagcccatgaacttcccagagagtggaacggcttgctcttctttctagaatgtccatgcacttgggttttaatcagcagttccctattattctgattttaagctgttcctgtgatgaacttagagacagcatcggtgtctgctgctgtgtccccaggtcttgtgtgggtggcacagatctgggcagttagatagtgctctgtgcctaaggtgaagccacactagggtgaagcctcacttccctgtttgagcaatgcagtgcctgctgcccgtgtgcatgaaggtacagccattcagataagtggaactattgagttacataaagaaaatagatttgcatttgtcaggcagacgtttatacaacaccacggtgcttttatacattgtgcttattttaataaaactgaaattctaaaaaaaaaaaaaaaaaa

SEQ ID NO: 2 - DNA, 2816 bp, Homo sapiens
  misc_feature: MKNK1
tctctgatcgggcccaccccctggatctagcaccgcctcttccgcgttctcggaggagcgatctgcaggtaggggtgcgcgcgaccgctccccggcgggagccagcgaaggtttccatgtcagaggccgatggagaactgaagattgccacctacgcacaaaggccattgagacacttcgtgtagctggaagacaccaacttcctgacaggagctttatttcatttgggatttcaagtttacagatggtatcttctcaaaagttggaaaaacctatagagatgggcagtagcgaaccccttcccatcgcagatggtgacaggaggaggaagaagaagcggaggggccgggccactgactccttgccaggaaagtttgaagatatgtacaagctgacctctgaattgcttggagagggagcctatgccaaagttcaaggtgccgtgagcctacagaatggcaaagagtatgccgtcaaaatcatcgagaaacaagcagggcacagtcggagtagggtgtttcgagaggtggagacgctgtatcagtgtcagggaaacaagaacattttggagctgattgagttctttgaagatgacacaaggttttacttggtctttgagaaattgcaaggaggttccatcttagcccacatccagaagcaaaagcacttcaatgagcgagaagccagccgagtggtgcgggacgttgctgctgcccttgacttcctgcataccaaagacaaagtctctctctgtcacctaggctggagtgctatggcgccatcagggctcactgcagccccaacctccctgggctccagtgatcctcccacctcagcctcccaagtagctgggactacaggcattgctcatcgtgatctgaaaccagaaaatatattgtgtgaatctccagaaaaggtgtctccagtgaaaatctgtgactttgacttgggcagtgggatgaaactgaacaactcctgtacccccataaccacaccagagctgaccaccccatgtggctctgcagaatacatggcccctgaggtagtggaggtcttcacggaccaggccacattctacgacaagcgctgtgacctgtggagcctgggcgtggtcctctacatcatgctgagtggctacccacccttcgtgggtcactgcggggccgactgtggctgggaccggggcgaggtctgcagggtgtgccagaacaagctgtttgaaagcatccaggaaggcaagtatgagtttcctgacaaggactgggcacacatctccagtgaagccaaagacctcatctccaagctcctggtgcgagatgcaaagcagagacttagcgccgcccaagttctgcagcacccatgggtgcaggggcaagctccagaaaagggactccccacgccgcaagtcctccagaggaacagcagcacaatggacctgacgctcttcgcagctgaggccatcgcccttaaccgccagctatctcagcacgaagagaacgaactagcagaggagccagaggcactagctgatggcctctgctccatgaagctttcccctccctgcaagtcacgcctggcccggagacgggccctggcccaggcaggccgtggtgaagacaggagcccgcccacagcactctgaaatgctccagtcacaccttataggccctaggcctggccaggcattgtcccctggaaacctgtgtggctaaagtctgctgagcaggcagcagcctctgctctgtggctccattcaggctttttcatctacgaaggccctgaggttcccatcaacccccatttccctagggtcctggaggaaaaagctttttccaaaggggttgtctttgaaaaggaaagcaatcacttctcactttgcataattgcctgcagcaggaacatctcttcactgggctccacctgctcacccgcctgcagatctgggatccagcctgctctcaccgctgtagctgtggcggctggggctgcagcctgcagggagaagcaagaagcatcagttgacagaggctgccgacacgtgcctcttccctctcttctctgtcaccctcctctggcggtccttccaccttcctctgtcctccggatgtcctctttgcccgtcttctcccttggctgagcaaagccatcccctcaattcagggaagggcaaggagccttcctcattcaggaaatcaaatcagtcttccggtctgcagcacggaaaagcacataatctttctttgctgtgactgaaatgtatccctcgtttatcatcccctttgtttgtgattgctgctaaagtcagtagtatcgtttttttaaaaaaaaagtttggtgtttttaaccatgctgttccagcaaagatgataccttaaactcccactgcaagcccatgaacttcccagagagtggaacggcttgctcttctttctagaatgtccatgcacttgggttttaatcagcagttccctattattctgattttaagctgttcctgtgatgaacttagagacagcatcggtgtctgctgctgtgtccccaggtcttgtgtgggtggcacagatctgggcagttagatagtgctctgtgcctaaggtgaagccacactagggtgaagcctcacttccctgtttgagcaatgcagtgcctgctgcccgtgtgcatgaaggtacagccattcagataagtggaactattgagttacataaagaaaatagatttgcatttgtcaggcagacgtttatacaacaccacggtgcttttatacattgtgcttattttaataaaactgaaattctaaaaaaaaaaaaaaaaaa

SEQ ID NO: 3 - DNA, 2607 bp, Homo sapiens
  misc_feature: MKNK1
tctctgatcgggcccaccccctggatctagcaccgcctcttccgcgttctcggaggagcgatctgcaggtttccatgtcagaggccgatggagaactgaagattgccacctacgcacaaaggccattgagacacttcgtgtagctggaagacaccaacttcctgacaggagctttatttcatttgggatttcaagtttacagatggtatcttctcaaaagttggaaaaacctatagagatgggcagtagcgaaccccttcccatcgcagatggtgacaggaggaggaagaagaagcggaggggccgggccactgactccttgccaggaaagtttgaagatatgtacaagctgacctctgaattgcttggagagggagcctatgccaaagttcaaggtgccgtgagcctacagaatggcaaagagtatgccgtcaaaatcatcgagaaacaagcagggcacagtcggagtagggtgtttcgagaggtggagacgctgtatcagtgtcagggaaacaagaacattttggagctgattgagttctttgaagatgacacaaggttttacttggtctttgagaaattgcaaggaggttccatcttagcccacatccagaagcaaaagcacttcaatgagcgagaagccagccgagtggtgcgggacgttgctgctgcccttgacttcctgcataccaaaggcattgctcatcgtgatctgaaaccagaaaatatattgtgtgaatctccagaaaaggtgtctccagtgaaaatctgtgactttgacttgggcagtgggatgaaactgaacaactcctgtacccccataaccacaccagagctgaccaccccatgtggctctgcagaatacatggcccctgaggtagtggaggtcttcacggaccaggccacattctacgacaagcgctgtgacctgtggagcctgggcgtggtcctctacatcatgctgagtggctacccacccttcgtgggtcactgcggggccgactgtggctgggaccggggcgaggtctgcagggtgtgccagaacaagctgtttgaaagcatccaggaaggcaagtatgagtttcctgacaaggactgggcacacatctccagtgaagccaaagacctcatctccaagctcctggtgcgagatgcaaagcagagacttagcgccgcccaagttctgcagcacccatgggtgcagggggaacagcagcacaatggacctgacgctcttcgcagctgaggccatcgcccttaaccgccagctatctcagcacgaagagaacgaactagcagaggagccagaggcactagctgatggcctctgctccatgaagctttcccctccctgcaagtcacgcctggcccggagacgggccctggcccaggcaggccgtggtgaagacaggagcccgcccacagcactctgaaatgctccagtcacaccttataggccctaggcctggccaggcattgtcccctggaaacctgtgtggctaaagtctgctgagcaggcagcagcctctgctctgtggctccattcaggctttttcatctacgaaggccctgaggttcccatcaacccccatttccctagggtcctggaggaaaaagctttttccaaaggggttgtctttgaaaaggaaagcaatcacttctcactttgcataattgcctgcagcaggaacatctcttcactgggctccacctgctcacccgcctgcagatctgggatccagcctgctctcaccgctgtagctgtggcggctggggctgcagcctgcagggagaagcaagaagcatcagttgacagaggctgccgacacgtgcctcttccctctcttctctgtcaccctcctctggcggtccttccaccttcctctgtcctccggatgtcctctttgcccgtcttctcccttggctgagcaaagccatcccctcaattcagggaagggcaaggagccttcctcattcaggaaatcaaatcagtcttccggtctgcagcacggaaaagcacataatctttctttgctgtgactgaaatgtatccctcgtttatcatcccctttgtttgtgattgctgctaaagtcagtagtatcgtttttttaaaaaaaaagtttggtgtttttaaccatgctgttccagcaaagatgataccttaaactcccactgcaagcccatgaacttcccagagagtggaacggcttgctcttctttctagaatgtccatgcacttgggttttaatcagcagttccctattattctgattttaagctgttcctgtgatgaacttagagacagcatcggtgtctgctgctgtgtccccaggtcttgtgtgggtggcacagatctgggcagttagatagtgctctgtgcctaaggtgaagccacactagggtgaagcctcacttccctgtttgagcaatgcagtgcctgctgcccgtgtgcatgaaggtacagccattcagataagtggaactattgagttacataaagaaaatagatttgcatttgtcaggcagacgtttatacaacaccacggtgcttttatacattgtgcttattttaataaaactgaaattctaaaaaaaaaaaaaaaaaa

SEQ ID NO: 4 - DNA, 2555 bp, Homo sapiens
  misc_feature: PRF1
gaagtgatgtgagtggtggctggtgcaaggagccacagtgggctgcctggggggctgatgccaccattccaggagcctcggtgaagagaggatatccatctgtgtagccgcttctctatacgggattccagagcccaagtgccccctgtctctgcagctccatggcagcccgtctgctcctcctgggcatccttctcctgctgctgcccctgcccgtccctgccccgtgccacacagccgcacgctcagagtgcaagcgcagccacaagttcgtgcctggtgcatggctggccggggagggtgtggacgtgaccagcctccgccgctcgggctccttcccagtggacacacaaaggttcctgcggcccgacggcacctgcaccctctgtgaaaatgccctacaggagggcaccctccagcgcctgcctctggcgctcaccaactggcgggcccagggctctggctgccagcgccatgtaaccagggccaaagtcagctccactgaagctgtggcccgggatgcggctcgtagcatccgcaacgactggaaggtcgggctggacgtgactcctaagcccaccagcaatgtgcatgtgtctgtggccggctcacactcacaggcagccaactttgcagcccagaagacccaccaggaccagtacagcttcagcactgacacggtggagtgccgcttctacagtttccatgtggtacacactcccccgctgcaccctgacttcaagagggccctcggggacctgccccaccacttcaacgcctccacccagcccgcctacctcaggcttatctccaactacggcacccacttcatccgggctgtggagctgggtggccgcatatcggccctcactgccctgcgcacctgcgagctggccctggaagggctcacggacaacgaggtggaggactgcctgactgtcgaggcccaggtcaacataggcatccacggcagcatctctgccgaagccaaggcctgtgaggagaagaagaagaagcacaagatgacggcctccttccaccaaacctaccgggagcgccactcggaagtggttggcggccatcacacctccattaacgacctgctgttcgggatccaggccgggcccgagcagtactcagcctgggtaaactcgctgcccggcagccctggcctggtggactacaccctggaacccctgcacgtgctgctggacagccaggacccgcggcgggaggcactgaggagggccctgagtcagtacctgacggacagggctcgctggagggactgcagccggccgtgcccaccagggcggcagaagagcccccgagacccatgccagtgtgtgtgccatggctcagcggtcaccacccaggactgctgccctcggcagaggggcctggcccagctggaggtgaccttcatccaagcatggggcctgtggggggactggttcactgccacggatgcctatgtgaagctcttctttggtggccaggagctgaggacgagcaccgtgtgggacaataacaaccccatctggtcagtgcggctggattttggggatgtgctcctggccacaggggggcccctgaggttgcaggtctgggatcaggactctggcagggacgatgacctccttggcacctgtgatcaggctcccaagtctggttcccatgaggtgagatgcaacctgaatcatggccacctaaaattccgctatcatgccaggtgcttgccccacctgggaggaggcacctgcctggactatgtcccccaaatgcttctgggggagcctccaggaaaccggagtggggccgtgtggtgagaacagtgagcttggaaaggaccagtatgcttggactgaaggggttctcacagtgggagccagggctgtcttcgtattcccattagaccaagcttgtccaacccgaggcccgcatgcggcccaggatggctttgaatgcggcccaacgcaaattcgcaaactttcttaaaacattatgagtttctttttgctattttttttttttttttagctcatcggctatcgttagtgctagtggattttacatgtggcccaacacaattcttcttccaacgtggcccagagaagccaaaagattggatacgcatcagacagatggaaaagggagattcagactgtttttcagggaggtggctgggtttacacgctaatcccgattcaccctgtccaaactgcctaagccctccgccattctcaagccctgcagtcacagctacacagatcacagcttcagccaggagctgggcagaaggccaagaggctgttcccaccaggctgctcagggctggtcttttaggacccttcccttgagccctctatggtgtggcaaagccttcattgccttaactggagccccatcagctccagctgctctgtcttctttgcccacaatgctttgcccctgagacaaatggaggcctgtcctgacctgtctcaccatgtacatagcttgataaagggccaataaatatgatgttatggtgaaaaaaaaaaaaaaaaaaaaaa

SEQ ID NO: 5 - DNA, 2529 bp, Homo sapiens
  misc_feature: PRF1
gaagtgatgtgagtggtggctggtgcaaggagccacagtgggctgcctggggggctgatgccaccattccaggagcctcggtgaagagaggatatccatctgtgtagccgcttctctatacgggattccagctccatggcagcccgtctgctcctcctgggcatccttctcctgctgctgcccctgcccgtccctgccccgtgccacacagccgcacgctcagagtgcaagcgcagccacaagttcgtgcctggtgcatggctggccggggagggtgtggacgtgaccagcctccgccgctcgggctccttcccagtggacacacaaaggttcctgcggcccgacggcacctgcaccctctgtgaaaatgccctacaggagggcaccctccagcgcctgcctctggcgctcaccaactggcgggcccagggctctggctgccagcgccatgtaaccagggccaaagtcagctccactgaagctgtggcccgggatgcggctcgtagcatccgcaacgactggaaggtcgggctggacgtgactcctaagcccaccagcaatgtgcatgtgtctgtggccggctcacactcacaggcagccaactttgcagcccagaagacccaccaggaccagtacagcttcagcactgacacggtggagtgccgcttctacagtttccatgtggtacacactcccccgctgcaccctgacttcaagagggccctcggggacctgccccaccacttcaacgcctccacccagcccgcctacctcaggcttatctccaactacggcacccacttcatccgggctgtggagctgggtggccgcatatcggccctcactgccctgcgcacctgcgagctggccctggaagggctcacggacaacgaggtggaggactgcctgactgtcgaggcccaggtcaacataggcatccacggcagcatctctgccgaagccaaggcctgtgaggagaagaagaagaagcacaagatgacggcctccttccaccaaacctaccgggagcgccactcggaagtggttggcggccatcacacctccattaacgacctgctgttcgggatccaggccgggcccgagcagtactcagcctgggtaaactcgctgcccggcagccctggcctggtggactacaccctggaacccctgcacgtgctgctggacagccaggacccgcggcgggaggcactgaggagggccctgagtcagtacctgacggacagggctcgctggagggactgcagccggccgtgcccaccagggcggcagaagagcccccgagacccatgccagtgtgtgtgccatggctcagcggtcaccacccaggactgctgccctcggcagaggggcctggcccagctggaggtgaccttcatccaagcatggggcctgtggggggactggttcactgccacggatgcctatgtgaagctcttctttggtggccaggagctgaggacgagcaccgtgtgggacaataacaaccccatctggtcagtgcggctggattttggggatgtgctcctggccacaggggggcccctgaggttgcaggtctgggatcaggactctggcagggacgatgacctccttggcacctgtgatcaggctcccaagtctggttcccatgaggtgagatgcaacctgaatcatggccacctaaaattccgctatcatgccaggtgcttgccccacctgggaggaggcacctgcctggactatgtcccccaaatgcttctgggggagcctccaggaaaccggagtggggccgtgtggtgagaacagtgagcttggaaaggaccagtatgcttggactgaaggggttctcacagtgggagccagggctgtcttcgtattcccattagaccaagcttgtccaacccgaggcccgcatgcggcccaggatggctttgaatgcggcccaacgcaaattcgcaaactttcttaaaacattatgagtttctttttgctattttttttttttttttagctcatcggctatcgttagtgctagtggattttacatgtggcccaacacaattcttcttccaacgtggcccagagaagccaaaagattggatacgcatcagacagatggaaaagggagattcagactgtttttcagggaggtggctgggtttacacgctaatcccgattcaccctgtccaaactgcctaagccctccgccattctcaagccctgcagtcacagctacacagatcacagcttcagccaggagctgggcagaaggccaagaggctgttcccaccaggctgctcagggctggtcttttaggacccttcccttgagccctctatggtgtggcaaagccttcattgccttaactggagccccatcagctccagctgctctgtcttctttgcccacaatgctttgcccctgagacaaatggaggcctgtcctgacctgtctcaccatgtacatagcttgataaagggccaataaatatgatgttatggtgaaaaaaaaaaaaaaaaaaaaaa

SEQ ID NO: 6 - DNA, 2589 bp, Homo sapiens
  misc_feature: TBX21
cggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttaaaaaaaaaaaaaaaaa

SEQ ID NO: 7 - DNA, 6472 bp, Homo sapiens
  misc_feature: TGFBR3
gagagcccccggtggggccggcggaggagcgcccttccccccgcgcgctgattgctgtggcgtcctgcccgtccccgcccgcgtgtgtgcgagggagggcgagtgcgccgggtcggcctgatgggggtaatcgagggtttcggggacgccgagcggcactttcctcttcccagcgagtgaaggagggcagtcggcggctctcgcgccccggccactttccctgcgcgattcccggagctccctgcaggaggtgagagtccccagcgggtccggatggcgtagttttgccgcggcgcagcagctgccggagctcgccgccgccgagcgctgggcggggaaacttgccgccgctttcctccaacttgctgcgggtggatctccgctggacacaccgcctccgaggcagtttgaaaattgcaaggagggactttaagactacttctgatttgcaaagattgtctgtgctctgagcaggctgaagtgactggacgagacgcactgttggagaaataaaaatgacttcccattatgtgattgccatctttgccctgatgagctcctgtttagccactgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtgatgggcattgcgtttgcagcctttgtgatcggagcactcctgacgggggccttgtggtacatctattctcacacaggggagacagcaggaaggcagcaagtccccacctccccgccagcctcggaaaacagcagtgctgcccacagcatcggcagcacgcagagcacgccttgctccagcagcagcacggcctagcccaacccagcccaacccggcccaacccagcccagcccagctcagctcagctactgccaagggcaggaccaatggctgagcctcgtgtccagactcagagggctggattttggttcccttgtaaagacagagtgaatttcagtataaagatcacccgttgtattcaccccacacccagggctagtataaacatgaccctgggcttctgtaccacactagaattcatgtgagaaagctaaaatggtggtcttctccaccagcccctcacaggcttgggggttttcaatgtgaaacacatgccagtttttaaaatgctgctttgtccaggtgagaacatccataatttggggccctgagttttacccagactcaaggagttggtaaagggttaatagccagatagtagaaccagtgaggagatgcggccaaagattctttatatctgaaccaagatgtaaaacaagaaatgctttgaggctttctaagcgatcctcctgtctaatttgcacctttgtctggatgcacacttctgaccttgctgccacaacctgtggggtctgatgtgtcccttgatgggtgctgccctcagggactgcaccctgacaagtgttaaggcaacattcctttcttgtgccctgggccaaaaccaatgctgatgaccttatcagcttcctgtttcttcccatacttgcatacaccactgcaaaatgtcttaatgcaaattttgtatttcttacaggcctacagaaattgaaaatgaccaaaatcaggaaccacagatttgtgcccattcctaatattttgttctgcaaattaatgtataatttgaggtgaaattcagttataaagtcaaggacgaatttgcacagtgatatatttctatgtgtatgcaagtacaagtatataatatgtcacctggcacattcattttctcagttgaagaagagaaaatttgaaaatgtccttatgcttttagagttgcaacttaagtatatttggtagggtgagtgtttccactcaaaatatgtcaacttaaaaaaaaataggccctttcataaaaaccaaactgtagcaagatgcaaatgcatggcaaatcctgtcggtctccagttggttatctgaatagtgtcaccaattccaccaagacagtgctgagattggaaaagggcactcatttggattgccttacttctcttgccttaaatatatcccatatatttaatatgtcaaaaagggcttgaggtgaatttcattaaatggaataatatgatgccactttgcagctaaaataagctcagtgatacctccttgttaaaaaaaaaaaaaaaaaaaaagaatgccaggggggaattatcatgctaaattattttactgctgaagatagcttcattgcaaattattctttttccagaatttcccccaaacaacttagaattccataagtagaacaagatagattctacccccaaaatattcataagcaaatcccactgcccttaaaataaggaagttatgcctggttgtctgtgaatggaacctcagtaggtaatcctgatgaagcttaaaaccttagggcagtgtctacatgggaaacaagtatgtggaaattgacaatcatttttaccaaatcgttgagtaataatatgacttacctgatagatatacacattcaataaggaacatttttttttttcaataaggaacattttaactaaaacattactttagggctgagatttccaggctatctacattgatctactgaaaaatttctagatttccatatacaaacgaagaagacatgagtttagagaatttttttcctttaatttttttttaacagaagggtatcagagtggaggacctagattacgctacctgtgtttgaaaatattgcgctttgcctaaccttcggtggaatgtattgtttcagcatattctatgtataaaaaagtttaaagatgtcttcaaagaagactagagtctttaagtcacttatagctatattttactacaaaatttgtgtataaagatatatttaagtgttttagataaatttaagttactccatatgaaatgtttaatttcagctactgtgaattctttcgatcagtcctttgcttttttaaaaaatcttttccatcgtgttaaaaagaaaattagctattaggagatattaaaagattgatgtcttagaaggacagggttaaaaccttctgtaatatatttaaatatatatttttaagagagatgctacattgccaatgattttataaataatttaaaattaatttacaaatttatagatgcaaaccaaaagatctttttaaaagagagagagaggaaaaaaatcaccttttatttgtttgggtctcattgtcagaattattattcagatcaagaaatctgtgtcgataaaacctaatctcatattggagagtgaatgtcgttaagtatttctgaaatgtttctaaaaaatgaacatgaaaaaaataaggtattttctccctagcttctcattacagtatttctaattgcttcacactttacagttttaatacaaagtacaagctctacttttataacgtgtactacaagtttctttaaaagtattgttgtatttcagctgaactcttgtatctgcagtcttttgttatttctgcaggctacacccttaatttgcagaggccattttgttgtttgtttgatgctgaagctgttgttaaaatgccccaaactccaaggcagattgtctgtataatacgctatagcttttcaccatgtggtgtttaccagctgttactctgtattttaaaaagccagagatttcttctgtctaaactgtttctatagcattttctaaacaaaaaaaatggtttttttctttgccttgtaataagattgtaaatccatttcctgcttgtgttcttagtgggcatgattgtgagtgtgtgtggggtgctcacacgcgtgccagtctttttgtactgtaactacctcatggtttgaatgatgattgtactgctggttgtgttaacagagcacattttgttgggtgagtcctatgtgatttttttcatgtttgttttatctgggggttgttcatgaaactgacagatgtttttctaaaaaggactattatcagtttccaaatacaatacttctctcttctggtttttcctgaatgagcctgattttgttgctggttttcatcatctatgagggtaaaacgagtacccttaaaatccctgtggccagtttgaacacccctgttgtattcttttctctgttcagtgtgtcagctattttgtgaagatgcttagatgtatagttttgataaccacagttttaaatcttttatctgtgcataataaaaagatatatatcagttattaaaaaaaaaa

SEQ ID NO: 8 - DNA, 6308 bp, Homo sapiens
  misc_feature: TGFBR3
tctttaagatttgtagctactaagaaagaaaggagctttttttccttgggccttcaaactgaaagaaccgcatgagcctgaagctgcgcatggtcttaacatcaggctgtgcaggaagaagctatctgcagatggatgccagcacacacaaggaagcagagctctggcaacattgagtcaaagcaaggacacaacatcagagggacggcagagaatccttgtgtgtagatctttggtggcagtttgaaaattgcaaggagggactttaagactacttctgatttgcaaagattgtctgtgctctgagcaggctgaagtgactggacgagacgcactgttggagaaataaaaatgacttcccattatgtgattgccatctttgccctgatgagctcctgtttagccactgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtgatgggcattgcgtttgcagcctttgtgatcggagcactcctgacgggggccttgtggtacatctattctcacacaggggagacagcaggaaggcagcaagtccccacctccccgccagcctcggaaaacagcagtgctgcccacagcatcggcagcacgcagagcacgccttgctccagcagcagcacggcctagcccaacccagcccaacccggcccaacccagcccagcccagctcagctcagctactgccaagggcaggaccaatggctgagcctcgtgtccagactcagagggctggattttggttcccttgtaaagacagagtgaatttcagtataaagatcacccgttgtattcaccccacacccagggctagtataaacatgaccctgggcttctgtaccacactagaattcatgtgagaaagctaaaatggtggtcttctccaccagcccctcacaggcttgggggttttcaatgtgaaacacatgccagtttttaaaatgctgctttgtccaggtgagaacatccataatttggggccctgagttttacccagactcaaggagttggtaaagggttaatagccagatagtagaaccagtgaggagatgcggccaaagattctttatatctgaaccaagatgtaaaacaagaaatgctttgaggctttctaagcgatcctcctgtctaatttgcacctttgtctggatgcacacttctgaccttgctgccacaacctgtggggtctgatgtgtcccttgatgggtgctgccctcagggactgcaccctgacaagtgttaaggcaacattcctttcttgtgccctgggccaaaaccaatgctgatgaccttatcagcttcctgtttcttcccatacttgcatacaccactgcaaaatgtcttaatgcaaattttgtatttcttacaggcctacagaaattgaaaatgaccaaaatcaggaaccacagatttgtgcccattcctaatattttgttctgcaaattaatgtataatttgaggtgaaattcagttataaagtcaaggacgaatttgcacagtgatatatttctatgtgtatgcaagtacaagtatataatatgtcacctggcacattcattttctcagttgaagaagagaaaatttgaaaatgtccttatgcttttagagttgcaacttaagtatatttggtagggtgagtgtttccactcaaaatatgtcaacttaaaaaaaaataggccctttcataaaaaccaaactgtagcaagatgcaaatgcatggcaaatcctgtcggtctccagttggttatctgaatagtgtcaccaattccaccaagacagtgctgagattggaaaagggcactcatttggattgccttacttctcttgccttaaatatatcccatatatttaatatgtcaaaaagggcttgaggtgaatttcattaaatggaataatatgatgccactttgcagctaaaataagctcagtgatacctccttgttaaaaaaaaaaaaaaaaaaaaagaatgccaggggggaattatcatgctaaattattttactgctgaagatagcttcattgcaaattattctttttccagaatttcccccaaacaacttagaattccataagtagaacaagatagattctacccccaaaatattcataagcaaatcccactgcccttaaaataaggaagttatgcctggttgtctgtgaatggaacctcagtaggtaatcctgatgaagcttaaaaccttagggcagtgtctacatgggaaacaagtatgtggaaattgacaatcatttttaccaaatcgttgagtaataatatgacttacctgatagatatacacattcaataaggaacatttttttttttcaataaggaacattttaactaaaacattactttagggctgagatttccaggctatctacattgatctactgaaaaatttctagatttccatatacaaacgaagaagacatgagtttagagaatttttttcctttaatttttttttaacagaagggtatcagagtggaggacctagattacgctacctgtgtttgaaaatattgcgctttgcctaaccttcggtggaatgtattgtttcagcatattctatgtataaaaaagtttaaagatgtcttcaaagaagactagagtctttaagtcacttatagctatattttactacaaaatttgtgtataaagatatatttaagtgttttagataaatttaagttactccatatgaaatgtttaatttcagctactgtgaattctttcgatcagtcctttgcttttttaaaaaatcttttccatcgtgttaaaaagaaaattagctattaggagatattaaaagattgatgtcttagaaggacagggttaaaaccttctgtaatatatttaaatatatatttttaagagagatgctacattgccaatgattttataaataatttaaaattaatttacaaatttatagatgcaaaccaaaagatctttttaaaagagagagagaggaaaaaaatcaccttttatttgtttgggtctcattgtcagaattattattcagatcaagaaatctgtgtcgataaaacctaatctcatattggagagtgaatgtcgttaagtatttctgaaatgtttctaaaaaatgaacatgaaaaaaataaggtattttctccctagcttctcattacagtatttctaattgcttcacactttacagttttaatacaaagtacaagctctacttttataacgtgtactacaagtttctttaaaagtattgttgtatttcagctgaactcttgtatctgcagtcttttgttatttctgcaggctacacccttaatttgcagaggccattttgttgtttgtttgatgctgaagctgttgttaaaatgccccaaactccaaggcagattgtctgtataatacgctatagcttttcaccatgtggtgtttaccagctgttactctgtattttaaaaagccagagatttcttctgtctaaactgtttctatagcattttctaaacaaaaaaaatggtttttttctttgccttgtaataagattgtaaatccatttcctgcttgtgttcttagtgggcatgattgtgagtgtgtgtggggtgctcacacgcgtgccagtctttttgtactgtaactacctcatggtttgaatgatgattgtactgctggttgtgttaacagagcacattttgttgggtgagtcctatgtgatttttttcatgtttgttttatctgggggttgttcatgaaactgacagatgtttttctaaaaaggactattatcagtttccaaatacaatacttctctcttctggtttttcctgaatgagcctgattttgttgctggttttcatcatctatgagggtaaaacgagtacccttaaaatccctgtggccagtttgaacacccctgttgtattcttttctctgttcagtgtgtcagctattttgtgaagatgcttagatgtatagttttgataaccacagttttaaatcttttatctgtgcataataaaaagatatatatcagttattaaaaaaaaaa

SEQ ID NO: 9 - DNA, 6475 bp, Homo sapiens
  misc_feature: TGFBR3
gagagcccccggtggggccggcggaggagcgcccttccccccgcgcgctgattgctgtggcgtcctgcccgtccccgcccgcgtgtgtgcgagggagggcgagtgcgccgggtcggcctgatgggggtaatcgagggtttcggggacgccgagcggcactttcctcttcccagcgagtgaaggagggcagtcggcggctctcgcgccccggccactttccctgcgcgattcccggagctccctgcaggaggtgagagtccccagcgggtccggatggcgtagttttgccgcggcgcagcagctgccggagctcgccgccgccgagcgctgggcggggaaacttgccgccgctttcctccaacttgctgcgggtggatctccgctggacacaccgcctccgaggcagtttgaaaattgcaaggagggactttaagactacttctgatttgcaaagattgtctgtgctctgagcaggctgaagtgactggacgagacgcactgttggagaaataaaaatgacttcccattatgtgattgccatctttgccctgatgagctcctgtttagccactgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgcagaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtgatgggcattgcgtttgcagcctttgtgatcggagcactcctgacgggggccttgtggtacatctattctcacacaggggagacagcaggaaggcagcaagtccccacctccccgccagcctcggaaaacagcagtgctgcccacagcatcggcagcacgcagagcacgccttgctccagcagcagcacggcctagcccaacccagcccaacccggcccaacccagcccagcccagctcagctcagctactgccaagggcaggaccaatggctgagcctcgtgtccagactcagagggctggattttggttcccttgtaaagacagagtgaatttcagtataaagatcacccgttgtattcaccccacacccagggctagtataaacatgaccctgggcttctgtaccacactagaattcatgtgagaaagctaaaatggtggtcttctccaccagcccctcacaggcttgggggttttcaatgtgaaacacatgccagtttttaaaatgctgctttgtccaggtgagaacatccataatttggggccctgagttttacccagactcaaggagttggtaaagggttaatagccagatagtagaaccagtgaggagatgcggccaaagattctttatatctgaaccaagatgtaaaacaagaaatgctttgaggctttctaagcgatcctcctgtctaatttgcacctttgtctggatgcacacttctgaccttgctgccacaacctgtggggtctgatgtgtcccttgatgggtgctgccctcagggactgcaccctgacaagtgttaaggcaacattcctttcttgtgccctgggccaaaaccaatgctgatgaccttatcagcttcctgtttcttcccatacttgcatacaccactgcaaaatgtcttaatgcaaattttgtatttcttacaggcctacagaaattgaaaatgaccaaaatcaggaaccacagatttgtgcccattcctaatattttgttctgcaaattaatgtataatttgaggtgaaattcagttataaagtcaaggacgaatttgcacagtgatatatttctatgtgtatgcaagtacaagtatataatatgtcacctggcacattcattttctcagttgaagaagagaaaatttgaaaatgtccttatgcttttagagttgcaacttaagtatatttggtagggtgagtgtttccactcaaaatatgtcaacttaaaaaaaaataggccctttcataaaaaccaaactgtagcaagatgcaaatgcatggcaaatcctgtcggtctccagttggttatctgaatagtgtcaccaattccaccaagacagtgctgagattggaaaagggcactcatttggattgccttacttctcttgccttaaatatatcccatatatttaatatgtcaaaaagggcttgaggtgaatttcattaaatggaataatatgatgccactttgcagctaaaataagctcagtgatacctccttgttaaaaaaaaaaaaaaaaaaaaagaatgccaggggggaattatcatgctaaattattttactgctgaagatagcttcattgcaaattattctttttccagaatttcccccaaacaacttagaattccataagtagaacaagatagattctacccccaaaatattcataagcaaatcccactgcccttaaaataaggaagttatgcctggttgtctgtgaatggaacctcagtaggtaatcctgatgaagcttaaaaccttagggcagtgtctacatgggaaacaagtatgtggaaattgacaatcatttttaccaaatcgttgagtaataatatgacttacctgatagatatacacattcaataaggaacatttttttttttcaataaggaacattttaactaaaacattactttagggctgagatttccaggctatctacattgatctactgaaaaatttctagatttccatatacaaacgaagaagacatgagtttagagaatttttttcctttaatttttttttaacagaagggtatcagagtggaggacctagattacgctacctgtgtttgaaaatattgcgctttgcctaaccttcggtggaatgtattgtttcagcatattctatgtataaaaaagtttaaagatgtcttcaaagaagactagagtctttaagtcacttatagctatattttactacaaaatttgtgtataaagatatatttaagtgttttagataaatttaagttactccatatgaaatgtttaatttcagctactgtgaattctttcgatcagtcctttgcttttttaaaaaatcttttccatcgtgttaaaaagaaaattagctattaggagatattaaaagattgatgtcttagaaggacagggttaaaaccttctgtaatatatttaaatatatatttttaagagagatgctacattgccaatgattttataaataatttaaaattaatttacaaatttatagatgcaaaccaaaagatctttttaaaagagagagagaggaaaaaaatcaccttttatttgtttgggtctcattgtcagaattattattcagatcaagaaatctgtgtcgataaaacctaatctcatattggagagtgaatgtcgttaagtatttctgaaatgtttctaaaaaatgaacatgaaaaaaataaggtattttctccctagcttctcattacagtatttctaattgcttcacactttacagttttaatacaaagtacaagctctacttttataacgtgtactacaagtttctttaaaagtattgttgtatttcagctgaactcttgtatctgcagtcttttgttatttctgcaggctacacccttaatttgcagaggccattttgttgtttgtttgatgctgaagctgttgttaaaatgccccaaactccaaggcagattgtctgtataatacgctatagcttttcaccatgtggtgtttaccagctgttactctgtattttaaaaagccagagatttcttctgtctaaactgtttctatagcattttctaaacaaaaaaaatggtttttttctttgccttgtaataagattgtaaatccatttcctgcttgtgttcttagtgggcatgattgtgagtgtgtgtggggtgctcacacgcgtgccagtctttttgtactgtaactacctcatggtttgaatgatgattgtactgctggttgtgttaacagagcacattttgttgggtgagtcctatgtgatttttttcatgtttgttttatctgggggttgttcatgaaactgacagatgtttttctaaaaaggactattatcagtttccaaatacaatacttctctcttctggtttttcctgaatgagcctgattttgttgctggttttcatcatctatgagggtaaaacgagtacccttaaaatccctgtggccagtttgaacacccctgttgtattcttttctctgttcagtgtgtcagctattttgtgaagatgcttagatgtatagttttgataaccacagttttaaatcttttatctgtgcataataaaaagatatatatcagttattaaaaaaaaaa

SEQ ID NO: 10 - DNA, 2234 bp, Homo sapiens
  misc_feature: IFNGR2
gttgactggaggcggaggttgcagtgagccgagatcgccccactgcactccagcctggtgactccgtctcaaaaaaaaggggaggggggcgggggagagttgaaagcttaatatgtactttgggggctattaaagcaaacatttcgactaaaggggcgaatcctcgaattgtgcgatcaagcacccgagaggagagttggggggggtcaggaggggtgggggctccagggaaagcccgggggtctgggccggggtctcgcggggcccttccggaaggatcgcggcccccgaaggtgggcgtcccgcggggctccagtctccaggacgttccgggaggctccgcgctctgggaggccggctgcgtggggtccccgcgctgcagccgcagaggccccccagggccgcggttcccggagcgggaaagtcccgcgcgggggcggtggcctcgggggcgggacggggcgggggcgggggcgcgggcggccgagccgaatcccctccaccgggacgccccgctgctgctcgggaagaggcgggccctgcgcgccctgcgctcgccatggcggtttgggcggcgacgtgagcggctccgcggaccccgagcggggccccggccgcgacctgagccgccgccgagcgcccggggccatgcgaccgacgctgctgtggtcgctgctgctgctgctcggagtcttcgccgccgccgccgcggccccgccagaccctctttcccagctgcccgctcctcagcacccgaagattcgcctgtacaacgcagagcaggtcctgagttgggagccagtggccctgagcaatagcacgaggcctgttgtctaccaagtgcagtttaaatacaccgacagtaaatggttcacggccgacatcatgtccataggggtgaattgtacacagatcacagcaacagagtgtgacttcactgccgccagtccctcagcaggcttcccaatggatttcaatgtcactctacgccttcgagctgagctgggagcactccattctgcctgggtgacaatgccttggtttcaacactatcggaatgtgactgtcgggcctccagaaaacattgaggtgaccccaggagaaggctccctcatcatcaggttctcctctccctttgacatcgctgatacctccacggcctttttttgttattatgtccattactgggaaaaaggaggaatccaacaggtcaaaggccctttcagaagcaactccatttcattggataacttaaaaccctccagagtgtactgtttacaagtccaggcacaactgctttggaacaaaagtaacatctttagagtcgggcatttaagcaacatatcttgctacgaaacaatggcagatgcctccactgagcttcagcaagtcatcctgatctccgtgggaacattttcgttgctgtcggtgctggcaggagcctgtttcttcctggtcctgaaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctttgaaccaaagcatgggcctagcccactggctccctggaagagatcaagccatcggagctgctagagttctgtctggactttccagagaccagtattcccttttgctgcctctaaaaggcctgtccctgcagacatgagagacagcaggtctcatgggggtgacaagctttttttttttttcttaaagaattttcaaaatcaaattccagaatgattttacggagatatcccaggaaaattaaggcttctcttaaacactaaaaaggcatgtaattgcttgttagcaaaatggatatgacacatctctgatacttttttcattattggttgggctgagcagtcagaagacctggtcgtcgtcttgactttggcaaatgagccggagccccttgggcaggtcacacaacctgtcccagcgagggacaccgagtggcccttcatgtacatccatggtgtgctggcttaaaatgtaattaatcttgtaaatatactcctagtaatttaagattttgtttttaaactggaaataaaagattgtatagtgcatgttttttaaagtctaaaaaaaaaaaaaa

SEQ ID NO: 11 - DNA, 2650 bp, Homo sapiens
  misc_feature: FYN
gccgcgctggtggcggcggcgcgtcgttgcagttgcgccatctgtcaggagcggagccggcgaggagggggctgccgcgggcgaggaggaggggtcgccgcgagccgaaggccttcgagacccgcccgccgcccggcggcgagagtagaggcgaggttgttgtgcgagcggcgcgtcctctcccgcccgggcgcgccgcgcttctcccagcgcaccgaggaccgcccgggcgcacacaaagccgccgcccgcgccgcaccgcccggcggccgccgcccgcgccagggagggattcggccgccgggccggggacaccccggcgccgccccctcggtgctctcggaaggcccaccggctcccgggcccgccggggaccccccggagccgcctcggccgcgccggaggagggcggggagaggaccatgtgagtgggctccggagcctcagcgccgcgcagtttttttgaagaagcaggatgctgatctaaacgtggaaaaagaccagtcctgcctctgttgtagaagacatgtggtgtatataaagtttgtgatcgttggcggacattttggaatttagataatgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagagagctgcaggtctctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtaaggcccgggtctgcggagagaggccttgtcccagaggctgccccacccctccccattagctttcaattccgtagccagctgctccccagcagcggaaccgcccaggatcagattgcatgtgactctgaagctgacgaacttccatggccctcattaatgacacttgtccccaaatccgaacctcctctgtgaagcattcgagacagaaccttgttatttctcagactttggaaaatgcattgtatcgatgttatgtaaaaggccaaacctctgttcagtgtaaatagttactccagtgccaacaatcctagtgctttccttttttaaaaatgcaaatcctatgtgattttaactctgtcttcacctgattcaactaaaaaaaaaaaagtattattttccaaaagtggcctctttgtctaaaacaataaaattttttttcatgttttaacaaaaaccaa

SEQ ID NO: 12 - DNA, 2073 bp, Homo sapiens
  misc_feature: FYN
gaatttagataatgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagaaagctgatggtttgtgttttaacttaactgtgattgcatcgagttgtaccccacaaacttctggattggctaaagatgcttgggaagttgcacgtcgttcgttgtgtctggagaagaagctgggtcaggggtgtttcgctgaagtgtggcttggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtaaggcccgggtctgcggagagaggccttgtcccagaggctgccccacccctccccattagctttcaattccgtagccagctgctccccagcagcggaaccgcccaggatcagattgcatgtgactctgaagctgacgaacttccatggccctcattaatgacacttgtccccaaatccgaacctcctctgtgaagcattcgagacagaaccttgttatttctcagactttggaaaatgcattgtatcgatgttatgtaaaaggccaaacctctgttcagtgtaaatagttactccagtgccaacaatcctagtgctttccttttttaaaaatgcaaatcctatgtgattttaactctgtcttcacctgattcaactaaaaaaaaaaaagtattattttccaaaagtggcctctttgtctaaaacaataaaattttttttcatgttttaacaaaaaccaa

SEQ ID NO: 13 - DNA, 2000 bp, Homo sapiens
  misc_feature: FYN
cagcgcaggtctgaggagctgagaagggaggcttacgtgaagggaatttagataatgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcaggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtaaggcccgggtctgcggagagaggccttgtcccagaggctgccccacccctccccattagctttcaattccgtagccagctgctccccagcagcggaaccgcccaggatcagattgcatgtgactctgaagctgacgaacttccatggccctcattaatgacacttgtccccaaatccgaacctcctctgtgaagcattcgagacagaaccttgttatttctcagactttggaaaatgcattgtatcgatgttatgtaaaaggccaaacctctgttcagtgtaaatagttactccagtgccaacaatcctagtgctttccttttttaaaaatgcaaatcctatgtgattttaactctgtcttcacctgattcaactaaaaaaaaaaaagtattattttccaaaagtggcctctttgtctaaaacaataaaattttttttcatgttttaacaaaaaccaaaaaaaaaaaaaaaaaaaaaaaaaaaaaaaaaaaaaaaaaa

SEQ ID NO: 14 - DNA, 1498 bp, Homo sapiens
  misc_feature: IL1B
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 15 - DNA, 10668 bp, Homo sapiens
  misc_feature: CFLAR
gaccacgcatggagaattttaccacccagagacacgcgagtggccctgtgcaagtttcaactgcgcggggggcggggaattccgcagacaggattctagaatttatgtcttgtgtggtaacatttcagccggtgggtggcggggattaggcgtgaagcggttcagcaggcagaggttctcggacgccctccggcgaagccacctgttgatgcttttgactttctgtccttgttcctcgtcccatctggagcatttccaattctggttttgcggagcagcaggtctgagcttgtccggcgagggtgggagttggtcccggcggagatccagtgggaagagccggcggctgcccgggcaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 16 - DNA, 1299 bp, Homo sapiens
  misc_feature: CFLAR
atactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggatgataacaccctatgcccattgtcctgatctgaaaattcttggaaattgttccatgtgattaacatggaactgcctctacttaatcattctgaatgattaaatcgtttcattttctaaatgtgttataatgtgtttagccctttcttgttgctgtatgtttagatgctttccaatcttttgttactactaataatgctataaaataaatatccttgtacttctttga

SEQ ID NO: 17 - DNA, 9734 bp, Homo sapiens
  misc_feature: CFLAR
accttggactgaaccactattgaagatttatttgataataatttgtaagttttaactcagtacctcccttctgcttttatagttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 18 - DNA, 10535 bp, Homo sapiens
  misc_feature: CFLAR
atactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 19 - DNA, 10222 bp, Homo sapiens
  misc_feature: CFLAR
atactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 20 - DNA, 10797 bp, Homo sapiens
  misc_feature: CFLAR
gggcacttggagttgtcttaggactgatgtctgaattaactaaatgaacttgtctggtttgcagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagggggacaattcccggaagtggaattacagagtcaaaggacatgcatttttcaagcctcggatgcatcttactagatgtcctataggatggtcatatcagctttataggagagtagctgtgtccctgaattctccctgacactgcatgctcttatatttcctcaagttttgacaatttgataggtgaaaagtggtatctgactgttcagatctggaaggctttgttatataaacatttttttaatgtttattggcaagaatacttttctaagagaaacatcagtgagctggtttccatttaagctgaatgaagccacaatgtacctcaagtataagattaactggcctttttcagttgcactctaattacaatttagaatgatgtttctgagccacctgtcaaatgcattctgggctgtacctctgcgtaccccaggaataaatctcatggccttctttacctggcctccttagtggtggcccagcaggaagcgggggttagagcaggagccactcagccttccaagatagatactccatgggccggtggtattactggccttttgagcccatccccatttgcatagatgatccacgtgggttatcatctggctggtatgttcccagagtgaaactcagcagccccttgagggaggggatggtggccatcaggccagagtattgcaagttagtttggatcatttgctaagcagcttgtggtgccttcagaaaggaacagtttcaaagaactttcacatctgttggctcatttcgccctaatgacagtcttctctttgatatttgcatggcattaaattttgcctttcttgttttctccagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 21 - DNA, 11061 bp, Homo sapiens
  misc_feature: CFLAR
atactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagggggacaattcccggaagtggaattacagagtcaaaggacatgcatttttcaagcctcggatgcatcttactagatgtcctataggatggtcatatcagctttataggagagtagctgtgtccctgaattctccctgacactgcatgctcttatatttcctcaagttttgacaatttgataggtgaaaagtggtatctgactgttcagatctggaaggctttgttatataaacatttttttaatgtttattggcaagaatacttttctaagagaaacatcagtgagctggtttccatttaagctgaatgaagccacaatgtacctcaagtataagattaactggcctttttcagttgcactctaattacaatttagaatgatgtttctgagccacctgtcaaatgcattctgggctgtacctctgcgtaccccaggaataaatctcatggccttctttacctggcctccttagtggtggcccagcaggaagcgggggttagagcaggagccactcagccttccaagatagatactccatgggccggtggtattactggccttttgagcccatccccatttgcatagatgatccacgtgggttatcatctggctggtatgttcccagagtgaaactcagcagccccttgagggaggggatggtggccatcaggccagagtattgcaagttagtttggatcatttgctaagcagcttgtggtgccttcagaaaggaacagtttcaaagaactttcacatctgttggctcatttcgccctaatgacagtcttctctttgatatttgcatggcattaaattttgcctttcttgttttctccagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 22 - DNA, 10640 bp, Homo sapiens
  misc_feature: CFLAR
atactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 23 - DNA, 1648 bp, Homo sapiens
  misc_feature: CD14
cagagaaggcttaggctcccgagtcaacagggcattcaccgcctggggcgcctgagtcatcaggacactgccaggagacacagaaccctagatgccctgcagaatccttcctgttacggtccccctccctgaaacatccttcattgcaatatttccaggaaaggaagggggctggctcggaggaagagaggtggggaggtgatcagggttcacagaggagggaactgaatgacatcccaggattacataaactgtcagaggcagccgaagagttcacaagtgtgaagcctggaagccggcgggtgccgctgtgtaggaaagaagctaaagcacttccagagcctgtccggagctcagaggttcggaagacttatcgaccatggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaagatccaagacagaataatgaatggactcaaactgccttggcttcaggggagtcccgtcaggacgttgaggacttttcgaccaattcaaccctttgccccacctttattaaaatcttaaacaacgggtcaaaaaaaaaaaaa

SEQ ID NO: 24 - DNA, 1561 bp, Homo sapiens
  misc_feature: CD14
aattctaccccccttggtgccaacagatgaggttcacaatctcttccacaaaacatgcagttaaatatctgaggatattcagggacttggatttggtggcaggagatcaacataaaccaagacaaggaagaagtcaaagaaatgaatcaagtagattctctgggatataagaggcagccgaagagttcacaagtgtgaagcctggaagccggcgggtgccgctgtgtaggaaagaagctaaagcacttccagagcctgtccggagctcagaggttcggaagacttatcgaccatggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaagatccaagacagaataatgaatggactcaaactgccttggcttcaggggagtcccgtcaggacgttgaggacttttcgaccaattcaaccctttgccccacctttattaaaatcttaaacaacgggtcaaaaaaaaaaaaa

SEQ ID NO: 25 - DNA, 1661 bp, Homo sapiens
  misc_feature: CD14
aattctaccccccttggtgccaacagatgaggttcacaatctcttccacaaaacatgcagttaaatatctgaggatattcagggacttggatttggtggcaggagatcaacataaaccaagacaaggaagaagtcaaagaaatgaatcaagtagattctctgggatataaggaaaggaagggggctggctcggaggaagagaggtggggaggtgatcagggttcacagaggagggaactgaatgacatcccaggattacataaactgtcagaggcagccgaagagttcacaagtgtgaagcctggaagccggcgggtgccgctgtgtaggaaagaagctaaagcacttccagagcctgtccggagctcagaggttcggaagacttatcgaccatggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaagatccaagacagaataatgaatggactcaaactgccttggcttcaggggagtcccgtcaggacgttgaggacttttcgaccaattcaaccctttgccccacctttattaaaatcttaaacaacgggtcaaaaaaaaaaaaa

SEQ ID NO: 26 - DNA, 1540 bp, Homo sapiens
  misc_feature: CD14
aattctaccccccttggtgccaacagatgaggttcacaatctcttccacaaaacatgcagttaaatatctgaggatattcagggacttggatttggtggcaggagatcaacataaaccaagacaaggaagaagtcaaagaaatgaatcaaaggcagccgaagagttcacaagtgtgaagcctggaagccggcgggtgccgctgtgtaggaaagaagctaaagcacttccagagcctgtccggagctcagaggttcggaagacttatcgaccatggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaagatccaagacagaataatgaatggactcaaactgccttggcttcaggggagtcccgtcaggacgttgaggacttttcgaccaattcaaccctttgccccacctttattaaaatcttaaacaacgggtcaaaaaaaaaaaaa

SEQ ID NO: 27 - DNA, 4704 bp, Homo sapiens
  misc_feature: TGFBR2
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 28 - DNA, 4629 bp, Homo sapiens
  misc_feature: TGFBR2
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 29 - DNA, 4353 bp, Homo sapiens
  misc_feature: MAPK14
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 30 - DNA, 4353 bp, Homo sapiens
  misc_feature: MAPK14
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 31 - DNA, 1431 bp, Homo sapiens
  misc_feature: MAPK14
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctgggtaagttgaccatatatcctcacctcatggatattgaattggttatgatataaattggggatttgaagaagagtttctccttttgaccaaataaagtaccattagttga

SEQ ID NO: 32 - DNA, 4274 bp, Homo sapiens
  misc_feature: MAPK14
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 33 - DNA, 751 bp, Homo sapiens
  misc_feature: GNLY
gtatctgtggtaaacccagtgacacgggggagatgacatacaaaaagggcaggacctgagaaagattaagctgcaggctccctgcccataaaacagggtgtgaaaggcatctcagcggctgccccaccatggctacctgggccctcctgctccttgcagccatgctcctgggcaacccaggtctggtcttctctcgtctgagccctgagtactacgacctggcaagagcccacctgcgtgatgaggagaaatcctgcccgtgcctggcccaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttacccagggcctcgtggccggagaaactgcccagcagatctgtgaggacctcaggttgtgtataccttctacaggtcccctctgagccctctcaccttgtcctgtggaagaagcacaggctcctgtcctcagatcccgggaacctcagcaacctctgccggctcctcgcttcctcgatccagaatccactctccagtctccctcccctgactccctctgctgtcctcccctctcacgagaataaagtgtcaagcaagattttaaaaaaaa

SEQ ID NO: 34 - DNA, 995 bp, Homo sapiens
  misc_feature: GNLY
gtatctgtggtaaacccagtgacacgggggagatgacatacaaaaagggcaggacctgagaaagattaagctgcaggctccctgcccataaaacagggtgtgaaaggcatctcagcggctgccccaccatggctacctgggccctcctgctccttgcagccatgctcctgggcaacccagcccctgcctccgcatctgcgtggtgaaggccattggccctcatcggtggatctgcgtttcctcgggcctacactgtctaggattgtgcggggctggtgagagaacaagatctcttctgtgttcaaggcagacttcctgccccctgcaccctgctctctcccaggccttgaggtcagtgtgagccccaagggcaagaacacttctggaagggagagtggatttggctgggccatctggatggaaggtctggtcttctctcgtctgagccctgagtactacgacctggcaagagcccacctgcgtgatgaggagaaatcctgcccgtgcctggcccaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttacccagggcctcgtggccggagaaactgcccagcagatctgtgaggacctcaggttgtgtataccttctacaggtcccctctgagccctctcaccttgtcctgtggaagaagcacaggctcctgtcctcagatcccgggaacctcagcaacctctgccggctcctcgcttcctcgatccagaatccactctccagtctccctcccctgactccctctgctgtcctcccctctcacgagaataaagtgtcaagcaagattttaaaaaaaa

SEQ ID NO: 35 - DNA, 25 bp, Artificial Sequence
  Probe h_CD14_Hs02621496_s1_FAM
ggaatggagacgcccacaggcgtgt

SEQ ID NO: 36 - DNA, 25 bp, Artificial Sequence
  Probe h_CFLAR_Hs01116280_m1_FAM
taagcaaggagaagagtttcttgga

SEQ ID NO: 37 - DNA, 25 bp, Artificial Sequence
  Probe h_IFNGR2_Hs00194264_m1_FAM
cggaatgtgactgtcgggcctccag

SEQ ID NO: 38 - DNA, 25 bp, Artificial Sequence
  Probe h_IL1B_Hs01555410_m1_FAM
gatgaagtgctccttccaggacctg

SEQ ID NO: 39 - DNA, 25 bp, Artificial Sequence
  Probe h_GNLY_Hs01120727_m1_FAM
taccttctacaggtcccctctgagc

SEQ ID NO: 40 - DNA, 25 bp, Artificial Sequence
  Probe h_MAPK14_315_Hs01051153_m1_FAM
tgtttcctggtacagaccatattaa

SEQ ID NO: 41 - DNA, 25 bp, Artificial Sequence
  Probe h_MKNK1_Hs00374376_m1_FAM
ccaccccatgtggctctgcagaata

SEQ ID NO: 42 - DNA, 25 bp, Artificial Sequence
  Probe h_PRF1_Hs00169473_m1_FAM
tgccgcttctacagtttccatgtgg

SEQ ID NO: 43 - DNA, 25 bp, Artificial Sequence
  Probe h_TBX21_Hs00894392_m1_FAM
acaatgtgacccagatgattgtgct

SEQ ID NO: 44 - DNA, 25 bp, Artificial Sequence
  Probe h_TGFBR2_Hs00234253_m1_FAM
tggctcaaccaccagggcatccaga

SEQ ID NO: 45 - DNA, 25 bp, Artificial Sequence
  Probe h_TGFBR3_Hs01114253_m1_FAM
cactgcaggtccagagcctggtgca